(19) United States
(12) Patent Application Publication
(10) Pub. No.: US 2023/0330167 A1
(43) Pub. Date: Oct. 19, 2023

(54) PHAGE AND TRANSDUCTION PARTICLES

(71) Applicant: SNIPR Biome ApS, Copenhagen Ø (DK)

(72) Inventor: Jakob Krause HAABER, Copenhagen Ø (DK)

(73) Assignee: SNIPR Biome ApS, Copenhagen Ø (DK)

(21) Appl. No.: 18/314,755

(22) Filed: May 9, 2023

Related U.S. Application Data
(63) Continuation of application No. 16/839,164, filed on Apr. 3, 2020, now abandoned.
Application No. 16/839,164 is a continuation of application No. 15/985,658, filed on May 21, 2018, now abandoned.

(30) Foreign Application Priority Data
Nov. 29, 2017 (GB) 1719896.1
May 17, 2018 (GB) 1808063.0

(51) Int. Cl. A61K 35/76; C12N 15/73; C12N 7/02; A61K 38/16; C07K 14/005

(52) U.S. Cl. CPC A61K 35/76; C12N 15/73; C12N 7/025; A61K 38/162; C07K 14/005; A61K 38/164; C12N 2795/00052; C12N 2795/10121; C12N 2795/10142; C12N 2795/10152; C12N 2310/20; C12N 2795/00042; C12N 2795/00032; C12N 2795/10132; C12N 2795/10151

(57) ABSTRACT

The invention relates to the production of phage and non-replicative transduction particles using DNAs (eg, plasmids and helper phage, mobile genetic elements (MGEs) or plasmids with chromosomally integrated helper phage genes), as well as the phage, helper phage, kits, compositions and methods involving these. The non-replicative transduction particles can be used to deliver antibacterial agents comprising a guided nuclease system.

### CROSS REFERENCE TO RELATED APPLICATION

This application claims priority benefit to United Kingdom Patent Application Nos. GB1719896.1 filed on Nov. 29, 2017 and GB1808063.0 filed on May 17, 2018, the contents of which are incorporated herein by reference in their entireties.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 786212000400SEQLIST.txt, date recorded: May 21, 2018, size: 71 KB).

### TECHNICAL FIELD

The invention relates to the production of phage using DNAs (eg, plasmids and helper phage, or plasmids with chromosomally integrated helper phage genes), as well as the phage, helper phage, kits, compositions and methods involving these.

### BACKGROUND

The use of helper phage to package phagemid DNA into phage virus particles is known. An example is the M13KO7 helper phage, a derivative of M13, used in E coli host cells. Other examples are R408 and CM13.

### SUMMARY OF THE INVENTION

The invention relates to the production of phage and provides:—

In a First Configuration

A kit comprising
a) A first DNA; and
b) One or more second DNAs;
Wherein
(i) the DNAs together comprise all phage structural protein genes required to produce a packaged phage particle comprising a copy of the first DNA;
(ii) the first DNA comprises none or at least one, but not all, of the genes; and wherein the one or more second DNAs comprise the remainder of the genes;
(iii) the first DNA comprises a phage packaging signal for producing the packaged phage particle; and
(iv) the second DNA is devoid of a nucleotide sequence (eg, a packaging signal) required for packaging the second DNA into phage particles;
wherein the DNAs are operable when co-existing in a host bacterium for producing packaged phage that comprise the first DNA, wherein the phage require the second DNA for replication thereof to produce further phage particles.
   There is also provided
   A method of producing phage, the method comprising expressing in a cell comprising the DNAs the phage protein genes, wherein packaged phage are produced that comprise the first DNA, wherein the phage require the second DNA for replication thereof to produce further phage particles.

In a Second Configuration

A population of helper phage, wherein the helper phage are capable of packaging first phage, wherein the first phage are different from the helper phage and the helper phage are incapable of self-replication.

In a Third Configuration

A composition comprising a population of first phage, wherein the first phage require helper phage according to the First Configuration for replication; and wherein less than [20%] of total phage comprised by the composition are such helper phage.

In a Fourth Configuration

A method of producing first phage, wherein the first phage require helper phage to replicate, the method comprising
(a) Providing DNA comprising a packaging signal;
(b) Introducing the DNA into a host bacterial cell;
(c) Wherein the host bacterial cell comprises helper phage or wherein helper phage are introduced into the bacterial cell simultaneously or sequentially with step (b);
(d) Wherein the helper phage are according to the invention;
(e) Causing or allowing the helper phage to produce phage proteins, wherein the packaging signal is recognised in the host cell, whereby first phage are produced using the proteins, the first phage packaging the DNA;
(f) Wherein helper phage replication in the host cell is inhibited or reduced, thereby limiting the availability of helper phage;
(g) Optionally lysing the host cell and obtaining the first phage;
(h) Thereby producing a composition comprising first phage which require the helper phage for replication, wherein propagation of first phage is prevented or reduced by the limitation of helper phage availability.

In a Fifth Configuration

A phage production system, for producing phage (eg, the first phage of any preceding claim) comprising a nucleotide sequence of interest (NSI-phage), the system comprising components (i) to (iii):—
(i) A first DNA;
(ii) A second DNA; and
(iii) a NSI-phage production factor (NPF) or an expressible nucleotide sequence that encodes a NPF;
Wherein
a) The first DNA encodes a helper phage (eg, said first helper phage recited in any preceding claim);
b) The second DNA comprises the nucleotide sequence of interest (NSI);
c) When the system is comprised by a bacterial host cell, helper phage proteins are expressed from the first DNA to form phage that package the second DNA in the presence of the NPF, thereby producing NSI-phage;
d) The system is devoid of a helper phage production factor (HPF) that is required for forming phage that package the first DNA, or is devoid of an expressible nucleotide sequence that encodes a functional HPF; or the system comprises a nucleotide sequence that comprises or encodes a functional HPF, the system further comprising means for targeted inactivation in the host cell of the HPF sequence to eliminate or minimise production of helper phage comprising the first DNA; and
Whereby the system is capable of producing a product comprising a population of NSI-phage, wherein each NSI-phage requires a said helper phage for propagation, wherein the NSI-phage in the product are not mixed with helper phage or less than [20%] of total phage comprised by the product are said helper phage.
   The invention also provides:_

A composition for use in antibacterial treatment of bacteria, the composition comprising an engineered mobile genetic element (MGE) that is capable of being mobilised in a first bacterial host cell of a first species or strain, the cell comprising a first phage genome, wherein in the cell the MGE is mobilised using proteins encoded by the phage and replication of first is inhibited, wherein the MGE encodes an antibacterial agent or encodes a component of such an agent.

A nucleic acid vector comprising the MGE integrated therein, wherein the vector is capable of transferring the MGE or a copy thereof into a host bacterial cell.

A non-self replicative transduction particle comprising said MGE or vector of the invention.

A composition comprising a plurality of transduction particles, wherein each particle comprises a MGE or vector according to the invention, wherein the transduction particles are capable of transferring the MGEs, or nucleic acid encoding the agent or component, or copies thereof into target bacterial cells, wherein
(i) target cells are killed by the antibacterial agent;
(ii) growth or proliferation of target cells is reduced; or
(iii) target cells are sensitised to an antibiotic, whereby the antibiotic is toxic to the cells.

A composition comprising a plurality of non-self replicative transduction particles, wherein each particle comprises a MGE or plasmid according to the invention, wherein the transduction particles are capable of transferring the MGEs, or nucleic acid encoding the agent or component, or copies thereof into target bacterial cells, wherein the agent is a CRISPR/Cas system and the component comprises a nucleic acid encoding a crRNA or a guide RNA that is operable with a Cas in a target bacterial cell to guide the Cas to a target nucleic acid sequence of the cell to modify the sequence, whereby
(i) target cells are killed by the antibacterial agent;
(ii) growth or proliferation of target cells is reduced; or
(iii) target cells are sensitised to an antibiotic, whereby the antibiotic is toxic to the cells.

A method of producing a plurality of transduction particles, the method comprising combining the composition of the invention with host bacterial cells of said first species, wherein the cells comprise the first phage, allowing a plurality of said MGEs to be introduced into host cells and culturing the host cells under conditions in which first phage-encoded proteins are expressed and MGE copies are packaged by first phage proteins to produce a plurality of transduction particles, and optionally separating the transduction particles from cells and obtaining a plurality of transduction particles separated from cells.

A bacterial host cell comprising a first phage and a MGE, vector or particle of the invention, wherein the agent is toxic to cells of the same species as the host cell, and wherein the host cell has been engineered so that the agent is not toxic to the host cell.

A bacterial host cell comprising a first phage, wherein the cell is comprised by a kit, the kit further comprising a composition of the invention, wherein the agent is toxic to cells of the same species as the host cell, and wherein the host cell has been engineered so that the agent is not toxic to the host cell.

A bacterial host cell comprising a first phage and a MGE, vector or particle of the invention, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

A bacterial host cell comprising a first phage, wherein the cell is comprised by a kit, the kit further comprising a composition of the invention, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

A bacterial host cell comprising a MGE, vector or particle of the invention and nucleic acid under the control of one or more inducible promoters, wherein the nucleic acid encodes all structural proteins necessary to produce a transduction particle that packages a copy of the MGE or plasmid, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

A plasmid comprising
(a) A nucleotide sequence encoding an antibacterial agent or component thereof for expression in target bacterial cells;
(b) A constitutive promoter for controlling the expression of the agent or component;
(c) An optional terS nucleotide sequence;
(d) An origin of replication (ori); and
(e) A phage packaging sequence (optionally pac, cos or a homologue thereof); and
   the plasmid being devoid of
(f) All nucleotide sequences encoding phage structural proteins necessary for the production of a transduction particle (optionally a phage), or the plasmid being devoid of at least one of such sequences; and
(g) Optionally terL.

A bacterial host cell comprising the genome of a helper phage that is incapable of self-replication, optionally wherein the genome is present as a prophage, and a plasmid according to the invention, wherein the helper phage is operable to package copies of the plasmid in transduction particles, wherein the particles are capable of infecting bacterial target cells to which the antibacterial agent is toxic.

A method of making a plurality of transduction particles, the method comprising culturing a plurality of host cells according to the invention, optionally inducing a lytic cycle of the helper phage, and incubating the cells under conditions wherein transducing particles comprising packaged copies of the plasmid are created, and optionally separating the particles from the cells to obtain a plurality of transduction particles.

A plurality of transduction particles obtainable by the method of the invention for use in medicine, eg, for treating or preventing an infection of a human or animal subject by target bacterial cells, wherein transducing particles are administered to the subject for infecting target cells and killing the cells using the antibacterial agent.

A method of making a plurality of transduction particles, the method comprising
(a) Producing host cells whose genomes comprise nucleic acid encoding structural proteins necessary to produce transduction particles that can package first DNA, wherein the genomes are devoid of a phage packaging signal, wherein the expression of the proteins is under the control of inducible promoter(s);
(b) Producing first DNA encoding an antibacterial agent or a component thereof, wherein the DNA comprises a phage packaging signal;
(c) Introducing the DNA into the host cells;
(d) Inducing production of the structural proteins in host cells, whereby transduction particles are produced that package the DNA;
(e) Optionally isolating a plurality of the transduction particles; and
(f) Optionally formulating the particles into a pharmaceutical composition for administration to a human or animal for medical use.

A plurality of transduction particles obtainable by the method.

### BRIEF DESCRIPTION OF THE FIGURES

FIG. 1 shows a genetic map of P2 genome.

FIG. 2 shows an exemplary saPI system (SaPIbov1).

FIG. 3 shows exemplary SaPIs.

### DETAILED DESCRIPTION

The invention relates to the production of phage using DNAs (eg, plasmids with helper phage), as well as the phage, helper phage, compositions and methods involving these. The invention finds utility, for example, for containing phage in environments ex vivo and in vivo, reducing the risk of acquisition of antibiotic resistance or other genes by phage, as well as controlling dosing of phage in an environment. The contamination of useful phage populations by helper phage may in examples also be restricted or eliminated, thereby controlling phage propagation and enhancing the proportion of desired phage in phage compositions, such as medicaments, herbicides and other agents where phage may usefully be used. Thus, the invention provides the following embodiments.

A kit comprising
a) A first DNA; and
b) One or more second DNAs;
Wherein
(i) the DNAs together comprise all phage structural protein genes required to produce a packaged phage particle comprising a copy of the first DNA;
(ii) the first DNA comprises none or at least one, but not all, of the genes; and wherein the one or more second DNAs comprise the remainder of the genes;
(iii) the first DNA comprises a phage packaging signal for producing the packaged phage particle; and
(iv) the second DNA is devoid of a nucleotide sequence required for packaging the second DNA into phage particles;
wherein the DNAs are operable when co-existing in a host bacterium for producing packaged phage that comprise the first DNA, wherein the phage require the second DNA for replication thereof to produce further phage particles.

For example the second DNA is devoid of a packaging signal for packaging second DNA. Additionally or alternatively, the second DNA is devoid of a nucleotide sequence required for replication of helper phage. Optionally, the nucleotide sequence encodes a sigma factor or comprises a sigma factor recognition site, a DNA polymerisation recognition site, or a promoter of a gene required for helper phage DNA replication when the second DNA is comprised by a helper prophage.

In an example, the second DNA is comprised by an M13 or M13-based helper phage. M13 encodes the following proteins required for phage packaging:—
a. pIII: host recognition
b. pV: coat protein
c. pVII, pVIII, pIX: membrane proteins
d. pI, pIV, pXI: Channel for translocating the phage to the extracellular space.

In this example, the second DNA is devoid of one or more of the genes coding for these proteins, eg, is devoid of a gene encoding pIII, a gene encoding pV, a gene encoding pVII, a gene encoding pVIII, a gene encoding pIX, a gene encoding pI, a gene encoding pIV and/or a gene encoding XI.

In an embodiment, the phage particle of (i) is capable of infecting a target bacterium, the phage comprising a nucleotide sequence of interest (NSI) that is capable of expressing a protein or RNA in the target bacterium, or wherein the NSI comprises a regulatory element that is operable in the target bacterium. In an example, the NSI is capable of recombination with the target cell chromosome or an episome comprised by the target cell to modify the chromosome or episome. Optionally, this is carried out in a method wherein the chromosome or episome is cut (eg, at a predetermined site using a guided nuclease, such as a Cas, TALEN, zinc finger or meganuclease; or a restriction endonuclease) and simultaneously or sequentially the cell is infected by a phage particle that comprises the first DNA, wherein the DNA is introduced into the cell and the NSI or a sequence thereof is introduced into the chromosome or episome at or adjacent the cut site. In an example the first DNA comprises one or more components of a CRISPR/Cas system operable to perform the cutting (eg, comprising at least a nucleotide sequence encoding a guide RNA or crRNA for targeting the site to be cut) and further comprising the NSI.

In an embodiment, the presence in the target bacterium of the NSI or its encoded protein or RNA mediates target cell killing, or downregulation of growth or propagation of target cells, or mediates switching off of expression of one or more RNA or proteins encoded by the target cell genome, or downregulation thereof.

In an embodiment, the presence in the target bacterium of the NSI or its encoded protein or RNA mediates upregulation of growth or propagation of the target cell, or mediates switching on of expression of one or more RNA or proteins encoded by the target cell genome, or upregulation thereof.

In an embodiment, the NSI encodes a component of a CRISPR/Cas system that is toxic to the target bacterium.

In an embodiment, the DNA is a first DNA as defined in any preceding paragraph.

In an embodiment, the first DNA is comprised by a vector (eg, a plasmid or shuttle vector).

In an embodiment, the second DNA is comprised by a vector (eg, a plasmid or shuttle vector), helper phage (eg, a helper phagemid) or is integrated in the genome of a host bacterial cell.

An embodiment provides a bacterial cell comprising the first and second DNAs. Optionally, the cell is devoid of a functional CRISPR/Cas system before transfer therein of a first DNA, eg, a first DNA comprising a component of a CRISPR/Cas system that is toxic to the target bacterium. An embodiment provides an antibacterial composition comprising a plurality of cells, wherein each cell is optionally according to this paragraph, for administration to a human or animal subject for medical use.

A method of producing phage is provided, the method comprising expressing in a host bacterial cell the phage protein genes, wherein packaged phage are produced that comprise the first DNA, wherein the phage require the second DNA for replication thereof to produce further phage particles. Optionally, the method comprises isolating the phage particles.

A composition comprising a population of phage particles obtainable by the method is provided for administration to a human or animal subject for treating an infection of target bacterial cells, wherein the phage are capable of infecting and killing the target cells.

A method of treating an environment ex vivo, the method comprising exposing the environment to a population of phage particles obtainable by the method is provided, wherein the environment comprises target bacteria and the phage infect and kill the target bacteria. In an example the subject is further administered an agent simultaneously or sequentially with the phage administration. In an example, the agent is a herbicide, pesticide, insecticide, plant fertilizer or cleaning agent.

Optionally, the method is for containing the treatment in the environment.

Optionally, the method is for controlling the dosing of the phage treatment in the environment.

Optionally, the method is for reducing the risk of acquisition of foreign gene sequence(s) by the phage in the environment.

A method of treating an infection of target bacteria in a human or animal subject is provided, the method comprising exposing the bacteria to a population of phage particles obtainable by the production method, wherein the phage infect and kill the target bacteria.

Optionally, the method for treating is for containing the treatment in the subject.

Optionally, the method for treating is for containing the treatment in the environment in which the subject exists.

Optionally, the method for treating is for controlling the dosing of the phage treatment in the subject.

Optionally, the method for treating is for reducing the risk of acquisition of foreign gene sequence(s) by the phage in the subject.

Optionally, the method for treating is for reducing the risk of acquisition of foreign gene sequence(s) by the phage in the environment in which the subject exists.

Optionally, target bacteria herein are comprised by a microbiome of the subject, eg, a gut microbiome. Alternatively, the microbiome is a skin, scalp, hair, eye, ear, oral, throat, lung, blood, rectal, anal, vaginal, scrotal, penile, nasal or tongue microbiome.

In an example the subject is further administered a medicament simultaneously or sequentially with the phage administration. In an example, the medicament is an antibiotic, antibody, immune checkpoint inhibitor (eg, an anti-PD-1, anti-PD-L1 or anti-CTLA4 antibody), adoptive cell therapy (eg, CAR-T therapy) or a vaccine.

In an example, the invention employs helper phage for packaging the phage nucleic acid of interest. Thus, the invention provides the following illustrative Aspects:—

1. A population of helper phage, wherein the helper phage are capable of packaging first phage nucleic acid to produce first phage particles, wherein the first phage are different from the helper phage and the helper phage are incapable themselves of producing helper phage particles.
2. A composition comprising a population of first phage, wherein the first phage require helper phage according to Aspect 1 for replication of first phage particles; and optionally wherein less than 20, 15, 10, 5, 4, 3, 2, 1, 0.5, 0.4, 0.2 or 0.1% of total phage particles comprised by the composition are particles of such helper phage.
In an example, the population comprises at least 10³, 10^{4,} 10⁵ or 10⁶ phage particles, as indicated a transduction assay, for example. To have a measure of the first phage concentration, for example, one can perform a standard transduction assay when the first phage genome contains an antibiotic marker. Thus, in this case the first phage are capable of infecting target bacteria and in a sample of 1 ml the population comprises at least 10³, 10^{4,} 10⁵ or 10⁶ transducing particles, which can be determined by infecting susceptible bacteria at a multiplicity of infection <0.1 and determining the number of infected cells by plating on a selective agar plate corresponding to the antibiotic marker in vitro at 20 to 37 degrees centigrade, eg, at 20 or 37 degrees centrigrade.
Optionally at least 99.9, 99.8, 99.7, 99.6, 99.5, 99.4, 99.3, 99.2, 99.1, 90, 85, 80, 70, 60, 50 or 40% of total phage particles comprised by the composition are particles of first phage.
In an example, the first phage genome comprises an f1 origin of replication.
In an example, the helper phage are E coli phage. In an example, the first phage are E coli, C difficile, Streptococcus, Klebsiella, Pseudomonas, Acinetobacter, Enterobacteriaceae, Firmicutes or Bacteroidetes phage. In an example, the helper phage are engineered M13 phage.
In an example, the first phage genome comprises a phagemid, wherein the phagemid comprises a packaging signal for packaging first phage particles in the presence of the helper phage.
The first phage particles may contain a nucleotide sequence of interest (NSI), eg, as defined herein, such as a NSI that encodes a component of a CRISPR/Cas system operable in target bacteria that can be infected by the first phage particles. Once inside the target bacteria, the first phage DNA is incapable of being packaged to form first phage particles in the absence of the helper phage. This usefully contains the activity of the first phage genome and its encoded products (proteins and/or nucleic acid), as well as limits or controls dosing of the NSI and its encoded products in an environment comprising the target bacteria that have been exposed to the first phage. This is useful, for example to control the medical treatment of an environment comprised by a human or animal subject, plant or other environment (eg, soil or a foodstuff or food ingredient).
3. The helper phage or composition of any preceding Aspect, wherein the genome of each first phage is devoid of genes encoding first phage structural proteins.
4. The composition of Aspect 2 or 3, wherein the composition comprises helper phage DNA.
5. The composition of Aspect 4, wherein the DNA comprises helper DNA fragments.
6. The helper phage or composition of any one preceding Aspect, wherein the helper phage are in the form of prophage.
Thus, the prophage is integrated in the chromosome of a host cell.
Examples of phage structural proteins are phage coat proteins, collar proteins and phage tail fibre proteins.
7. The composition of any one of Aspects 2 or 3, wherein the composition comprises no helper phage DNA comprising a sequence of 20 contiguous nucleotides or more, eg, no helper phage DNA.
This can be determined, for example, using DNA probes (designed on the basis of the known helper phage genome sequence) with PCR, as is conventional. In an example, the composition may comprise residual helper prophage DNA, but essentially otherwise is devoid of helper DNA.
8. The composition of any one of Aspects 2 to 5 and 7, wherein the helper phage are capable of infecting host bacteria and the composition does not comprise host bacteria.
9. The composition of any one of Aspects 2 to 8, wherein the composition is a lysate of host bacterial cells, wherein the lysate comprises helper prophage DNA, eg, such DNA comprises 20 contiguous nucleotides or more of helper phage DNA.
10. The composition of any one of Aspects 2 to 8, wherein the composition is a lysate of host bacterial cells, wherein the lysate has been processed (eg, filtered) to remove all or some helper phage DNA; or the composition is a lysate of host bacterial cells that is devoid of cellular material.
11. The composition of any one of Aspects 2 to 10, wherein the composition does not comprise helper phage particles.
12. The composition of any one of Aspects 2 to 11, wherein at least 95% (eg, 100%) of phage particles comprised by the composition are first phage particles.
In another embodiment, the composition comprises second phage particles, wherein the second phage are different from the first phage and are not helper phage.
13. The composition of any one of Aspects 2 to 12, wherein the population comprises at least 10³, 10⁴, 10⁵ or 10⁶ phage particles, as indicated in a transduction assay.
14. The helper phage or composition of any preceding Aspect, wherein the first phage are capable of replicating in host bacteria in the presence of the helper phage (eg, helper prophage), wherein the first phage comprise antibacterial means for killing target bacteria of a first strain or species, wherein the target bacteria are of a different strain or species and the antibacterial means is not operable to kill the target bacteria.
15. A composition comprising a population of phage, the population comprising
(a) A first sub-population of first phage that require a helper phage for packaging the first phage;
(b) A second sub-population of phage comprising the helper phage, wherein the helper phage are as recited in any preceding Aspect.
   16. The helper phage or composition of any preceding Aspect, wherein the helper phage are phagemids.
   17. A composition comprising
(a) A population of helper phage as recited in any preceding Aspect; and
(b) A population of nucleic acid vectors comprising vector DNA that comprises a first phage packaging signal;
(c) wherein the helper phage are capable of packaging the vector DNA to produce first phage.
   18. The composition of Aspect 17, wherein the vectors are phage.
   19. The composition of Aspect 17, wherein the vectors are plasmids or phagemids.
   20. The composition of Aspect 19, the vectors are shuttle vectors (eg, pUC vectors) that can be replicated in first bacteria, wherein the vectors can further be replicated and packaged into first phage in second bacteria (host bacteria) in the presence of the helper phage, wherein the first bacteria are of a strain or species that is different to the strain or species of the host bacteria.
   21. The composition of Aspect 21, wherein the first phage are capable of infecting third bacteria of a strain or species that is different to the second (and optionally also the first) bacteria.
   22. The composition of any one of Aspects 17 to 21, wherein the first phage are capable of replicating in host bacteria in the presence of the helper phage (eg, helper prophage), wherein the first phage comprise antibacterial means for killing target bacteria of a first strain or species, wherein the host bacteria are of a different strain or species and the antibacterial means is not operable to kill the host bacteria.
   23. The helper phage or composition of any preceding Aspect, wherein the genome is devoid of a packaging signal (eg, SEQ ID NO:1 below), wherein the helper phage are incapable of self-replication.
   24. The helper phage or composition of Aspect 24, wherein the signal is a pac or cos sequence.
   25. The helper phage or composition of any preceding Aspect, wherein the helper phage genome is capable of replication in a host cell.
   Thus, the genome is capable of nucleic acid replication but not packaging of helper phage.
   26. The helper phage or composition of any one of Aspects 1 to 24, wherein the genome is devoid of a nucleotide sequence required for production of helper phage particles.
   27. The helper phage or composition of Aspect 26, wherein the nucleotide sequence encodes a sigma factor (eg, sigma-70) or comprises a sigma factor recognition site, a DNA polymerisation recognition site, or a promoter of a gene required for helper phage DNA replication.
   28. The helper phage or composition of any preceding Aspect, wherein the helper phage are temperate phage.
   29. The helper phage or composition of any one of Aspects 1 to 27, wherein the helper phage are lytic phage.
   30. The helper phage or composition of any preceding Aspect, wherein the first phage are capable of infecting target bacteria, the first phage comprising a nucleotide sequence of interest (NSI) that is capable of expressing a protein or RNA (eg, gRNA or crRNA) in target bacteria, or wherein the NSI comprises a regulatory element that is operable in target bacteria.
   31. The helper phage or composition of Aspect 30, wherein the presence in target bacteria of the NSI or its encoded protein or RNA mediates target cell killing, or downregulation of growth or propagation of target cells, or mediates switching off of expression of one or more RNA or proteins encoded by the target cell genomes, or downregulation thereof.
   32. The helper phage or composition of Aspect 30, wherein the presence in target bacteria of the NSI or its encoded protein or RNA mediates upregulation of growth or propagation of target cells, or mediates switching on of expression of one or more RNA or proteins encoded by the target cell genomes, or upregulation thereof.
   33. An antibacterial composition according to any one of Aspects 2 to 32, wherein the first phage are capable of infecting target bacteria and each first phage comprises engineered antibacterial means for killing target bacteria.
   By use of the term “engineered” it will be readily apparent to the skilled addressee that the relevant means has been introduced and is not naturally-occurring in the phage. For example, the means is recombinant, artificial or synthetic.
   34. The composition of Aspect 14, 22 or 33, wherein the antibacterial means comprises one or more components of a CRISPR/Cas system.
   35. The composition of claim 34, wherein the component(s) comprise (i) a DNA sequence encoding a guide RNA (eg, a single guide RNA) or comprising a CRISPR array for producing guide RNA, wherein the guide RNA is capable of targeting the genome of target bacteria; (ii) a Cas nuclease-encoding DNA sequence; and/or (iii) a DNA sequence encoding one or more components of Cascade.
   In an example, a Cas herein is a Cas9. In an example, a Cas herein is a Cas3. The Cas may be identical to a Cas encoded by the target bacteria.
   36. The composition of any one of Aspects 14, 22 or 33 to 35, wherein the antibacterial means comprises a nucleic acid encoding a guided nuclease, such as a Cas nuclease, TALEN, zinc finger nuclease or meganuclease.
   37. The helper phage or composition of any preceding Aspect, wherein the helper phage is for use in medicine practised on a human or animal subject, or the composition is a pharmaceutical composition for use in medicine practised on a human or animal subject.
   In an example, the animal is a livestock or companion pet animal (eg, a cow, pig, goat, sheep, horse, dog, cat or rabbit). In an example, the animal is an insect (an insect at any stage of its lifecycle, eg, egg, larva or pupa). In an example, the animal is a protozoan. In an example, the animal is a cephalopod.
   38. The composition of any one of Aspects 2 to 36, wherein the composition is a herbicide, pesticide, food or beverage processing agent, food or beverage additive, petrochemical or fuel processing agent, water purifying agent, cosmetic additive, detergent additive or environmental (eg, soil) additive or cleaning agent.
   39. The helper phage or composition of any one of Aspects 1 to 37 for use in a contained method of treating a disease or condition of a human or animal subject, wherein the disease or condition is mediated by the target bacteria and the target bacteria are comprised by the subject, the method comprising administering the composition to the subject, whereby the target bacteria are exposed to the antibacterial means and killed and propagation of the first phage is contained.
   The inability of the first phage to self-replicate and to require helper phage or second DNA to do this usefully provides containment in the location (eg, gut) of action of the composition and/or in the environment of the subject, eg, when exposed to secretions such as urine and faeces of the subject that otherwise may contain replicated first phage. Inability of the helper phage or second DNA to self-package limits availability of factors required by the first phage to form packaged particles, hence providing containment by limiting first phage propagation. This may be useful, for example, to contain an antibacterial activity provided by the first phage, such as a CRISPR/Cas killing principle.
   40. A bacterial cell or a plurality of bacterial cells comprising the helper phage or composition of any preceding Aspect, wherein the first phage are capable of replication in the presence of the helper phage in the cell.
   The cell may, for example, act as a carrier for the genome of the first phage, wherein the first phage DNA is capable of horizontal transfer from the carrier to the target bacteria once the carrier bacteria have been administered to an environment to be treated, eg, a soil or a human gut or other environment described herein. In an example, the environment is comprised by a human or animal subject and the carrier are commensal or probiotic in the subject. For example the carrier bacteria are Lactobacillus (eg, L reuteri or L lactis), E coli or Streptococcus (eg, S thermophilus) bacteria. The horizontal transfer can be transfer of a plasmid (such as a conjugative plasmid) to the target bacteria or first phage infection of the target bacteria, wherein the first phage have been prior packaged in the carrier. The use of a carrier is useful too for oral administration or other routes where the carrier can provide protection for the phage, helper or composition from the acid stomach or other harsh environments in the subject. Furthermore, the carrier can be formulated into a beverage, for example, a probiotic drink, eg, an adapted Yakult (trademark), Actimel (trademark), Kevita (trademark), Activia (trademark), Jarrow (trademark) or similar drink for human consumption.
   41. The cell(s) of Aspect 40 for administration to a human or animal subject for medical use, comprising killing target bacteria using first phage, wherein the target bacteria mediate as disease or condition in the subject.
   In an example, when the subject is a human, the subject is not an embryo.
   42. The cell(s) of Aspect 41, wherein the cell(s) comprises helper phage and is symbiotic or probiotic in the subject.
   43. A method of killing target bacteria in an environment, optionally wherein the method is not practised on a human or animal body, wherein the method comprises exposing the environment to the cell(s) according to Aspect 42, or a composition obtained or obtainable by the method of any one of Aspects 57 to 65, wherein the environment is or has been exposed to first phage or said vectors to produce first phage in the presence of the helper phage, wherein the first phage are capable of replication in the environment and kill target bacteria.
   44. The cell(s) or method of any one of Aspects 40 to 43, wherein the cell is an E coli, Lactobacillus (eg, L lactis or retueri) or Streptococcus (eg, thermophilus) cell.
   45. The cell(s) or method of Aspects 40 to 44 wherein the subject is administered or has been administered a cell comprising first phage.
   46. The composition of any one of Aspects 2 to 45 in combination with a target bacterial cell wherein the first phage are capable of infecting the target bacterial cell.
   47. Use of the helper phage, composition or cell(s) of any one of Aspects 1 to 42 and 44 to 46, or a composition obtained or obtainable by the method of any one of Aspects 57 to 65, in the manufacture of an antibacterial agent that kills target bacteria, for containment of the antibacterial in an environment, eg, containment ex vivo; or containment in a human or animal subject comprising the environment.
   48. Use of the helper phage, composition or cell(s) of any one of Aspects 1 to 42 and 44 to 46, or a composition obtained or obtainable by the method of any one of Aspects 57 to 65, in the manufacture of an antibacterial agent that kills the target bacteria, for reducing the risk of acquisition by the first phage of foreign genes.
   For example, this is useful for reducing the risk of antibiotic resistance genes by the phage, such as when the phage are in the presence of other phage or plasmids in the environment.
   49. Use of the helper phage, composition or cell(s) of any one of Aspects 1 to 42 and 44 to 46, or a composition obtained or obtainable by the method of any one of Aspects 57 to 65, in the manufacture of an antibacterial agent that kills the target bacteria, for reducing the risk of acquisition by the first phage of one or more antibiotic resistance genes.
   50. A method of reducing the risk of acquisition by first phage of foreign genes, the method comprising
(a) Providing the composition of any one of Aspects 2 to 42 and 44 to 46, or a composition obtained or obtainable by the method of any one of Aspects 57 to 65; and
(b) Exposing target bacteria to the composition, wherein the first phage infect the target bacteria;
(c) wherein the helper phage are incapable of self-replication and propagation of first phage is thereby limited, wherein propagation of first phage is prevented or reduced, thereby reducing the risk of acquisition of first phage of foreign genes (eg, antibiotic resistance genes).
   51. A method of containing an antibacterial activity in an environment (e.g., ex vivo), the method comprising
(a) Providing an antibacterial composition according to any one of Aspects 2 to 42 and 44 to 46, or a composition obtained or obtainable by the method of any one of Aspects 57 to 65; and
(b) Exposing target bacteria in the environment to the composition, wherein the bacteria are exposed to the first phage and antibacterial means and are killed;
(c) wherein the helper phage are incapable of self-replication and propagation of first phage is thereby limited, wherein propagation of first phage is prevented or reduced, thereby containing the antibacterial activity.
   52. A method of controlling the dosing of first phage in an environment (e.g., ex vivo), the method comprising
(a) Providing an antibacterial composition according to any one of Aspects 2 to 42 and 44 to 46, or a composition obtained or obtainable by the method of any one of Aspects 57 to 65; and
(b) Exposing target bacteria in the environment to the composition, wherein the bacteria are infected by first phage;
(c) wherein the helper phage are incapable of self-replication and propagation of first phage is thereby limited, wherein propagation of first phage is prevented or reduced, thereby controlling dosing of first phage in the environment.
   53. The method of any one of Aspects 43 to 45, 51 and 52, or the use of Aspect 47, wherein the environment is a human or animal microbiome, e.g., a gut microbiome.
   54. The method of any one of Aspects 43 to 45, 51 and 52, or the use of Aspect 47, wherein the environment is a microbiome of soil; a plant, part of a part (e.g., a leaf, fruit, vegetable or flower) or plant product (e.g., pulp); water; a waterway; a fluid; a foodstuff or ingredient thereof; a beverage or ingredient thereof; a medical device; a cosmetic; a detergent; blood; a bodily fluid; a medical apparatus; an industrial apparatus; an oil rig; a petrochemical processing, storage or transport apparatus; a vehicle or a container.
   55. The method of any one of Aspects 43 to 45, 51 and 52, or the use of Aspect 47, wherein the environment is an ex vivo bodily fluid (e.g., urine, blood, blood product, sweat, tears, sputum or spit), bodily solid (e.g., faeces) or tissue of a human or animal subject that has been administered the composition.
   56. The method of any one of Aspects 43 to 45, 51 and 52, or the use of Aspect 47, wherein the environment is an in vivo bodily fluid (e.g., urine, blood, blood product, sweat, tears, sputum or spit), bodily solid (e.g., faeces) or tissue of a human or animal subject that has been administered the composition.
   57. A method of producing first phage, wherein the first phage require helper phage to replicate, the method comprising
(a) Providing DNA comprising a packaging signal;
(b) Introducing the DNA into a host bacterial cell;
(c) Wherein the host bacterial cell comprises helper phage or wherein helper phage are introduced into the bacterial cell simultaneously or sequentially with step (b);
(d) Wherein the helper phage are according to any preceding Aspect;
(e) Causing or allowing the helper phage to produce phage coat proteins, wherein the packaging signal is recognised in the host cell, whereby first phage are produced using the proteins, the first phage packaging the DNA;
(f) Wherein helper phage particle production in the host cell is inhibited or reduced, thereby limiting the availability of helper phage particles;
(g) Optionally lysing the host cell and obtaining the first phage;
(h) Thereby producing a composition comprising first phage which require the helper phage for replication, wherein further production of first phage particles is prevented or reduced by the limitation of helper phage availability in the composition.
   In an embodiment, the DNA is comprised by a phagemid or cloning vector (eg, a shuttle vector, eg, a pUC vector).
   There may be a modest amount of helper phage DNA replication to enable first phage protein production efficiently, or should replication of helper phage DNA may be eliminated totally eliminated.
   58. The method of Aspect 57, wherein in (c) the helper phage are prophage integrated in the bacterial cell chromosome.
   59. The method of Aspect 59, wherein (e) comprises inducing replication of helper phage DNA and/or expression of the proteins, eg, using UV, mitomycin.
   60. The method of any one of Aspects 57 to 59, wherein (g) comprises further separating the first phage from cellular material or helper phage DNA.
   61. The method of any one of Aspects 57 to 60, wherein the composition comprises a population of first phage particles, wherein the composition does not comprise helper phage DNA and/or particles.
   62. The method of any one of Aspects 57 to 61, wherein the DNA of (a) comprises engineered antibacterial means for killing target bacteria.
   63. The method of Aspect 62, wherein the antibacterial means comprises one or more components of a CRISPR/Cas system.
   64. The method of Aspect 63, wherein the component(s) comprise (i) a DNA sequence encoding a guide RNA (eg, a single guide RNA) or comprising a CRISPR array for producing guide RNA, wherein the guide RNA is capable of targeting the genome of target bacteria; (ii) a Cas (eg, Cas9, Cas3, Cpf1, CasX or CasY) nuclease-encoding DNA sequence; and/or (iii) a DNA sequence encoding one or more components of Cascade (eg, CasA).
   65. The method of any one of Aspects 62 to 64, wherein the antibacterial means comprises a nucleic acid encoding a guided nuclease, such as a Cas nuclease, TALEN, zinc finger nuclease or meganuclease.
   66. The helper phage, composition or cell(s) of any one of Aspects 1 to 42 and 44 to 46, or a composition obtained or obtainable by the method of any one of Aspects 57 to 65, for antibacterial treatment of target bacteria in a human or animal subject whereby the antibacterial treatment is contained in the subject.
   67. The helper phage, composition or cell(s) of any one of Aspects 1 to 42 and 44 to 46, or a composition obtained or obtainable by the method of any one of Aspects 57 to 65, for antibacterial treatment of target bacteria in a gut of a human or animal subject whereby the antibacterial activity in one or more bodily excretions of the subject is reduced.
   This is useful as a safety measure to reduce or eliminate first phage activity outside the subject.
   68. The helper phage, composition or cell(s) of Aspect 67, wherein the antibacterial activity in one or more bodily excretions of the subject is eliminated.
   69. The helper phage, composition or cell(s) of any one of Aspects 1 to 42 and 44 to 46, or a composition obtained or obtainable by the method of any one of Aspects 57 to 65, for controlling the dosing of antibacterial treatment of target bacteria in a human or animal subject, eg, in the gut of the subject.
   Usefully, propagation of the first phage is restricted or eliminated, so dosing in the subject can be controlled, or even pre-determined within a narrow expected range. This is useful, for example, for medicaments comprising the first phage or composition, and may be aid approval of such medicines before FDA and similar authorities.
   Alternatively, the dosing is dosing of an environment, such as soil etc disclosed herein, wherein limitation of the first phage or composition activity is also desirable to limit spread of activities in natural and other terrains.
   70. The helper phage, composition or cell(s) of any one of Aspects 1 to 42 and 44 to 46, or a composition obtained or obtainable by the method of any one of Aspects 57 to 65, for fixing the dosing of antibacterial treatment of target bacteria in a human or animal subject, eg, in the gut of the subject.
   71. A phage production system, for producing phage (eg, the first phage of any preceding Aspect) comprising a nucleotide sequence of interest (NSI-phage), the system comprising components (i) to (iii):—
(a) A first DNA;
(b) A second DNA; and
(c) a NSI-phage production factor (NPF) or an expressible nucleotide sequence that encodes a NPF;
Wherein
(d) The first DNA encodes a helper phage (eg, said first helper phage recited in any preceding Aspect);
(e) The second DNA comprises the nucleotide sequence of interest (NSI);
(f) When the system is comprised by a bacterial host cell, helper phage proteins are expressed from the first DNA to form phage that package the second DNA in the presence of the NPF, thereby producing NSI-phage; and
(g) The system is devoid of a helper phage production factor (HPF) that is required for forming helper phage particles that package the first DNA, or is devoid of an expressible nucleotide sequence that encodes a functional HPF; or the system comprises a nucleotide sequence that comprises or encodes a functional HPF, the system further comprising means for targeted inactivation in the host cell of the HPF sequence to eliminate or minimise production of helper phage comprising the first DNA;
   Whereby the system is capable of producing a product comprising a population of NSI-phage, wherein each NSI-phage requires a said helper phage for propagation, optionally wherein the NSI-phage in the product are not mixed with helper phage or less than 20% of total phage comprised by the product are said helper phage.
   The invention includes within its concept relatively low level of helper phage particle production if there is a residual capability of helper phage to replicate to produce particles, such as for example in the case that a helper phage packaging signal or other HPF nucleotide sequence in the helper phage genome is mutated (eg, by deletion, substitution or addition of nucleotides therein) to knock down the ability to form phage particles. Preferably, there is no production of helper phage particles, such as by deleting all or part of the sequence from the helper phage genome or inactivating the sequence.
   72. A method of producing first phage, wherein the first phage require helper phage to replicate, the method comprising
(a) Providing in host cells the system of Aspect 71;
(b) Causing or allowing the helper phage proteins to be produced, whereby the second DNA is packaged to produce first phage; and
(c) Optionally lysing the host cells and obtaining a composition comprising first phage.
   73. The method of Aspect 72, wherein step (c) comprises separating the first phage from cellular material.
   74. The method of Aspect 72 or 73, wherein the composition comprises a population of first phage, wherein less than 20, 10, 5, 4, 3, 2, 1, 0.5 or 0.1% of total phage comprised by the composition are helper phage.
   75. The method of any one of Aspects 72 to 74, wherein the second DNA comprises engineered antibacterial means for killing target bacteria.
   76. The method of Aspect 75, wherein the antibacterial means comprises one or more components of a CRISPR/Cas system.
   77. The method of Aspect 76 wherein the component(s) comprise (i) a DNA sequence encoding a guide RNA (eg, a single guide RNA) or comprising a CRISPR array for producing guide RNA, wherein the guide RNA is capable of targeting the genome of target bacteria; (ii) a Cas nuclease-encoding DNA sequence; and/or (iii) a DNA sequence encoding one or more components of Cascade.
   78. The method of any one of Aspects 75 to 77, wherein the antibacterial means comprises a nucleic acid encoding a guided nuclease, such as a Cas nuclease, TALEN, zinc finger nuclease or meganuclease
   79. The system of method of any one of Aspects 71 to 78, wherein the first phage are capable of infecting target bacteria, the NSI being capable of expressing a protein or RNA in target bacteria, or wherein the NSI comprises a regulatory element that is operable in target bacteria.
   80. The system or method of Aspect 79, wherein the presence in target bacteria of the NSI or its encoded protein or RNA mediates target cell killing, or downregulation of growth or propagation of target cells, or mediates switching off of expression of one or more RNA or proteins encoded by the target cell genomes, or downregulation thereof.
   81. The system or method of Aspect 79, wherein the presence in target bacteria of the NSI or its encoded protein or RNA mediates upregulation of growth or propagation of target cells, or mediates switching on of expression of one or more RNA or proteins encoded by the target cell genomes, or upregulation thereof.
   82. The system of method of any one of Aspects 71 to 81, wherein each of the NPF and HPF is a packaging signal, eg, SEQ ID NO:1 or a sequence that is at least 70, 80, 90, 95, 96, 97, 98 or 99% identical thereto, or is a homologue from a different species.
   83. The system of method of Aspect 82, wherein each signal is a pac or cos sequence, or is a homologue.
   84. The system of method of any one of Aspects 71 to 81, wherein the HPF is a nucleotide sequence required for replication of helper phage.
   85. The system of method of any one of Aspects 71 to 81, wherein the HPF encodes a sigma factor (eg, sigma-70) or comprises a sigma factor recognition site, a DNA polymerisation recognition site, or a promoter of a gene required for helper phage DNA replication, a helper phage integrase, a helper phage excissionase or a helper phage origin of replication,
   86. A composition comprising a population of first phage obtainable by the method of any one of Aspects 72 to 85, wherein the genome of each first phage is devoid of genes encoding phage proteins.
   87. The composition of Aspect 86, wherein the first phage comprise antibacterial means as recited in any one of Aspects 75 to 78.
   88. The composition of Aspect 87, comprising DNA identical to the first DNA or fragments thereof.
   89. The composition of Aspect 88, wherein the DNA of the composition is identical to the first DNA and is devoid of a helper phage packaging signal.
   90. The composition of any one of Aspects 86 to 89 for antibacterial treatment of target bacteria in a human or animal subject whereby the antibacterial treatment is contained in the subject.
   91. The composition of any one of Aspects 86 to 89 for antibacterial treatment of target bacteria in a gut of a human or animal subject whereby the antibacterial activity in one or more bodily excretions of the subject is reduced.
   92. The composition of Aspect 91, wherein the antibacterial activity in one or more bodily excretions of the subject is eliminated.
   93. The composition of any one of Aspects 86 to 89 for controlling the dosing of antibacterial treatment of target bacteria in a human or animal subject, eg, in the gut of the subject.
   94. The composition of any one of Aspects 86 to 89 for fixing the dosing of antibacterial treatment of target bacteria in a human or animal subject, eg, in the gut of the subject.
   95. An isolated DNA comprising all structural protein genes of a helper phage genome that are required for producing phage particles, wherein the DNA is devoid of a helper phage production factor (HPF) that is required for producing packaged helper phage, optionally wherein the DNA comprises one or more promoters for expression of the genes when the DNA is integrated in the genome of a host bacterial cell.
   96. The DNA of Aspect 95, wherein the DNA is devoid of any phage packaging signals.
   97. The DNA of Aspect 95 or 96, wherein the HPF is a sigma factor-encoding nucleotide sequence or comprises a sigma factor recognition site, a DNA polymerisation recognition site, a promoter of a gene required for helper phage DNA replication, a helper phage integrase-encoding nucleotide sequence, a helper phage excissionase-encoding nucleotide sequence or a helper phage origin of replication.
   98. The DNA of any one of Aspects 95 to 97, wherein the DNA comprises a nucleotide sequence encoding a CRISPR/Cas system repressor.
   99. The DNA of any one of Aspects 95 to 98, wherein the DNA is integrated in the chromosome of a host bacterial cell, wherein the genes are expressible in the host cell.
   100. The DNA of Aspect 99, wherein the cell is devoid of an active CRISPR/Cas system.
   101. The DNA of any one of Aspects 95 to 100 in combination with a second DNA, wherein the second DNA comprises the HPF.
   102. The DNA of any one of Aspects 95 to 100 in combination with a second DNA, wherein the second DNA comprises a phage packaging signal and optionally the first DNA is devoid of a phage packaging signal.
   103. The DNA of Aspect 101 or 102, wherein the second DNA is comprised by a phagemid or a plasmid (eg, a shuttle vector).

In an example, the kit, DNA(s), first phage, helper phage or composition is comprised by a medical container, eg, a syringe, vial, IV bag, inhaler, eye dropper or nebulizer. In an example, the kit, DNA(s), first phage, helper phage or composition is comprised by a sterile container. In an example, the kit, DNA(s), first phage, helper phage or composition is comprised by a medically-compatible container. In an example, the kit, DNA(s), first first phage, helper phage or composition is comprised by a fermentation vessel, eg, a metal, glass or plastic vessel.

In an example, the kit, DNA(s), first phage, helper phage or composition is comprised by a medicament, e,g in combination with instructions or a packaging label with directions to administer the medicament by oral, IV, subcutaneous, intranasal, intraocular, vaginal, topical, rectal or inhaled administration to a human or animal subject. In an example, the kit, DNA(s), first phage, helper phage or composition is comprised by an oral medicament formulation. In an example, the kit, DNA(s), first phage, helper phage or composition is comprised by an intranasal or ocular medicament formulation. In an example, the kit, DNA(s), first phage, helper phage or composition is comprised by a personal hygiene composition (eg, shampoo, soap or deodorant) or cosmetic formulation. In an example, the kit, DNA(s), first phage, helper phage or composition is comprised by a detergent formulation. In an example, the kit, DNA(s), first phage, helper phage or composition is comprised by a cleaning formulation, eg, for cleaning a medical or industrial device or apparatus. In an example, the kit, DNA(s), first phage, helper phage or composition is comprised by foodstuff, foodstuff ingredient or foodstuff processing agent. In an example, the kit, DNA(s), first phage, helper phage or composition is comprised by beverage, beverage ingredient or beverage processing agent. In an example, the kit, DNA(s), first phage, helper phage or composition is comprised by a medical bandage, fabric, plaster or swab. In an example, the kit, DNA(s), first phage, helper phage or composition is comprised by a herbicide or pesticide. In an example, the kit, DNA(s), first phage, helper phage or composition is comprised by an insecticide.

In an example, the first phage is a is a Corticoviridae, Cystoviridae, Inoviridae, Leviviridae, Microviridae, Myoviridae, Podoviridae, Siphoviridae, or Tectiviridae virus. In an example, the helper phage is a is a Corticoviridae, Cystoviridae, Inoviridae, Leviviridae, Microviridae, Myoviridae, Podoviridae, Siphoviridae, or Tectiviridae virus. In an example, the helper phage is a filamentous M13, a Noviridae, a tailed phage (eg, a Myoviridae, Siphoviridae or Podoviridae), or a non-tailed phage (eg, a Tectiviridae).

In an example, both the first and helper phage are Corticoviridae. In an example, both the first and helper phage are Cystoviridae. In an example, both the first and helper phage are Inoviridae. In an example, both the first and helper phage are Leviviridae. In an example, both the first and helper phage are Microviridae. In an example, both the first and helper phage are Podoviridae. In an example, both the first and helper phage are Siphoviridae. In an example, both the first and helper phage are Tectiviridae.

In an example, the CRISPR/Cas component(s) are component(s) of a Type I CRISPR/Cas system. In an example, the CRISPR/Cas component(s) are component(s) of a Type II CRISPR/Cas system. In an example, the CRISPR/Cas component(s) are component(s) of a Type III CRISPR/Cas system. In an example, the CRISPR/Cas component(s) are component(s) of a Type IV CRISPR/Cas system. In an example, the CRISPR/Cas component(s) are component(s) of a Type V CRISPR/Cas system. In an example, the CRISPR/Cas component(s) comprise a Cas9-encoding nucleotide sequence (eg, S pyogenes Cas9, S aureus Cas9 or S thermophilus Cas9). In an example, the CRISPR/Cas component(s) comprise a Cas3-encoding nucleotide sequence (eg, E coli Cas3, C difficile Cas3 or Salmonella Cas3). In an example, the CRISPR/Cas component(s) comprise a Cpf-encoding nucleotide sequence. In an example, the CRISPR/Cas component(s) comprise a CasX-encoding nucleotide sequence. In an example, the CRISPR/Cas component(s) comprise a CasY-encoding nucleotide sequence.

In an example, the first DNA, first phage or vector encode a CRISPR/Cas component or protein of interest from a nucleotide sequence comprising a promoter that is operable in the target bacteria.

In an example, the host bacteria and/or target bacteria are E coli. In an example, the host bacteria and/or target bacteria are C difficile (eg, the vector is a shuttle vector operable in E coli and the host bacteria are C difficile). In an example, the host bacteria and/or target bacteria are Streptococcus, such as S thermophilus (eg, the vector is a shuttle vector operable in E coli and the host bacteria are Streptococcus). In an example, the host bacteria and/or target bacteria are Pseudomonas, such as P aeruginosa (eg, the vector is a shuttle vector operable in E coli and the host bacteria are P aeruginosa). In an example, the host bacteria and/or target bacteria are Klebsiella (eg, the vector is a shuttle vector operable in E coli and the host bacteria are Klebsiella). In an example, the host bacteria and/or target bacteria are Salmonella, eg, S typhimurium (eg, the vector is a shuttle vector operable in E coli and the host bacteria are Salmonella).

Optionally, host and/or target bacteria is a gram negative bacterium (eg, a spirilla or vibrio). Optionally, host and/or target bacteria is a gram positive bacterium. Optionally, host and/or target bacteria is a Mycoplasma, chlamydiae, spirochete or Mycobacterium. Optionally, host and/or target bacteria is a Streptococcus (eg, pyogenes or thermophilus). Optionally, host and/or target bacteria is a Staphylococcus (eg, aureus, eg, MRSA). Optionally, host and/or target bacteria is an E. coli (eg, O157: H7) host, eg, wherein the Cas is encoded by the vector or an endogenous host Cas nuclease activity is de-repressed. Optionally, host and/or target bacteria is a Pseudomonas (eg, aeruginosa). Optionally, host and/or target bacteria is a Vibrio (eg, cholerae (eg, 0139) or vulnificus). Optionally, host and/or target bacteria is a Neisseria (eg, gonorrhoeae or meningitidis). Optionally, host and/or target bacteria is a Bordetella (eg, pertussis). Optionally, host and/or target bacteria is a Haemophilus (eg, influenzae). Optionally, host and/or target bacteria is a Shigella (eg, dysenteriae). Optionally, host and/or target bacteria is a Brucella (eg, abortus). Optionally, host and/or target bacteria is a Francisella host. Optionally, host and/or target bacteria is a Xanthomonas host. Optionally, host and/or target bacteria is a Agrobacterium host. Optionally, host and/or target bacteria is a Erwinia host. Optionally, host and/or target bacteria is a Legionella (eg, pneumophila). Optionally, host and/or target bacteria is a Listeria (eg, monocytogenes). Optionally, host and/or target bacteria is a Campylobacter (eg, jejuni). Optionally, host and/or target bacteria is a Yersinia (eg, pestis). Optionally, host and/or target bacteria is a Borrelia (eg, burgdorferi). Optionally, host and/or target bacteria is a Helicobacter (eg, pylori). Optionally, host and/or target bacteria is a Clostridium (eg, difficile or botulinum). Optionally, host and/or target bacteria is a Ehrlichia (eg, chaffeensis). Optionally, host and/or target bacteria is a Salmonella (eg, typhi or enterica, eg, serotype typhimurium, eg, DT 104). Optionally, host and/or target bacteria is a Chlamydia (eg, pneumoniae). Optionally, host and/or target bacteria is a Parachlamydia host. Optionally, host and/or target bacteria is a Corynebacterium (eg, amycolatum). Optionally, host and/or target bacteria is a Klebsiella (eg, pneumoniae). Optionally, host and/or target bacteria is an Enterococcus (eg, faecalis or faecim, eg, linezolid-resistant). Optionally, host and/or target bacteria is an Acinetobacter (eg, baumannii, eg, multiple drug resistant).

Further examples of target cells and targeting of antibiotic resistance in such cells using the present invention are as follows:—
1. Optionally the target bacteria are Staphylococcus aureus cells, eg, resistant to an antibiotic selected from methicillin, vancomycin, linezolid, daptomycin, quinupristin, dalfopristin and teicoplanin.
2. Optionally the target bacteria are Pseudomonas aeuroginosa cells, eg, resistant to an antibiotic selected from cephalosporins (eg, ceftazidime), carbapenems (eg, imipenem or meropenem), fluoroquinolones, aminoglycosides (eg, gentamicin or tobramycin) and colistin.
3. Optionally the target bacteria are Klebsiella (eg, pneumoniae) cells, eg, resistant to carbapenem.
4. Optionally the target bacteria are Streptococcus (eg, thermophilus, pneumoniae or pyogenes) cells, eg, resistant to an antibiotic selected from erythromycin, clindamycin, beta-lactam, macrolide, amoxicillin, azithromycin and penicillin.
5. Optionally the target bacteria are Salmonella (eg, serotype Typhi) cells, eg, resistant to an antibiotic selected from ceftriaxone, azithromycin and ciprofloxacin.
6. Optionally the target bacteria are Shigella cells, eg, resistant to an antibiotic selected from ciprofloxacin and azithromycin.
7. Optionally the target bacteria are Mycobacterium tuberculosis cells, eg, resistant to an antibiotic selected from Resistance to isoniazid (INH), rifampicin (RMP), fluoroquinolone, amikacin, kanamycin and capreomycin and azithromycin.
8. Optionally the target bacteria are Enterococcus cells, eg, resistant to vancomycin.
9. Optionally the target bacteria are Enterobacteriaceae cells, eg, resistant to an antibiotic selected from a cephalosporin and carbapenem.
10. Optionally the target bacteria are E. coli cells, eg, resistant to an antibiotic selected from trimethoprim, itrofurantoin, cefalexin and amoxicillin.
11. Optionally the target bacteria are Clostridium (eg, difficile) cells, eg, resistant to an antibiotic selected from fluoroquinolone antibiotic and carbapenem.
12. Optionally the target bacteria are Neisseria gonorrhoea cells, eg, resistant to an antibiotic selected from cefixime (eg, an oral cephalosporin), ceftriaxone (an injectable cephalosporin), azithromycin and tetracycline.
13. Optionally the target bacteria are Acinetobacter baumannii cells, eg, resistant to an antibiotic selected from beta-lactam, meropenem and a carbapenem.
14. Optionally the target bacteria are Campylobacter cells, eg, resistant to an antibiotic selected from ciprofloxacin and azithromycin.
15. Optionally, the target cell(s) produce Beta (β)-lactamase.
16. Optionally, the target cell(s) are bacterial cells that are resistant to an antibiotic recited in any one of examples 1 to 14.

### Mobile Genetic Elements, Genomic Islands, Pathogenicity Islands Etc.

Genetic variation of bacteria and archaea can be achieved through mutations, rearrangements and horizontal gene transfers and recombinations. Increasing genome sequence data have demonstrated that, besides the core genes encoding house-keeping functions such as essential metabolic activities, information processing, and bacterial structural and regulatory components, a vast number of accessory genes encoding antimicrobial resistance, toxins, and enzymes that contribute to adaptation and survival under certain environmental conditions are acquired by horizontal gene transfer of mobile genetic elements (MGEs). Mobile genetic elements are a heterogeneous group of molecules that include plasmids, bacteriophages, genomic islands, chromosomal cassettes, pathogenicity islands, and integrative and conjugative elements. Genomic islands are relatively large segments of DNA ranging from 10 to 200 kb often integrated into tRNA gene clusters flanked by 16-20 bp direct repeats. They are recognized as discrete DNA segments acquired by horizontal gene transfer since they can differ from the rest of the chromosome in terms of GC content (% G+C) and codon usage.

Pathogenicity islands (PTIs) are a subset of horizontally transferred genetic elements known as genomic islands. There exists a particular family of highly mobile PTIs in Staphylococcus aureus that are induced to excise and replicate by certain resident prophages. These PTIs are packaged into small headed phage-like particles and are transferred at frequencies commensurate with the plaque-forming titer of the phage. This process is referred to as the SaPI excision replication-packaging (ERP) cycle, and the high-frequency SaPI transfer is referred to as SaPI-specific transfer (SPST) to distinguish it from classical generalized transduction (CGT). The SaPIs have a highly conserved genetic organization that parallels that of bacteriophages and clearly distinguishes them from all other horizontally acquired genomic islands. The SaPI1-encoded and SaPIbov2-encoded integrases are used for both excision and integration of the corresponding elements, and it is assumed that the same is true for the other SaPIs. Phage 80α can induce several different SaPIs, including SaPI1, SaPI2, and SaPIbov1, whereas φ11 can induce SaPIbov1 but neither of the other two SaPIs.

Reference is made to “Staphylococcal pathogenicity island DNA packaging system involving cos-site packaging and phage-encoded HNH endonucleases”, Quiles-Puchalt et al, PNAS Apr. 22, 2014. 111 (16) 6016-6021. Staphylococcal pathogenicity islands (SaPIs) are highly mobile and carry and disseminate superantigen and other virulence genes. It was reported that SaPIs hijack the packaging machinery of the phages they victimise, using two unrelated and complementary mechanisms. Phage packaging starts with the recognition in the phage DNA of a specific sequence, termed “pac” or “cos” depending on the phage type. The SaPI strategies involve carriage of the helper phage pac- or cos-like sequences in the SaPI genome, which ensures SaPI packaging in full-sized phage particles, depending on the helper phage machinery. These strategies interfere with phage reproduction, which ultimately is a critical advantage for the bacterial population by reducing the number of phage particles.

Staphylococcal pathogenicity islands (SaPIs) are the prototypical members of a widespread family of chromosomally located mobile genetic elements that contribute substantially to intra- and interspecies gene transfer, host adaptation, and virulence. The key feature of their mobility is the induction of SaPI excision and replication by certain helper phages and their efficient encapsidation into phage-like infectious particles. Most SaPIs use the headful packaging mechanism and encode small terminase subunit (TerS) homologs that recognize the SaPI-specific pac site and determine SaPI packaging specificity. Several of the known SaPIs do not encode a recognizable TerS homolog but are nevertheless packaged efficiently by helper phages and transferred at high frequencies. Quiles-Puchalt et al report that one of the non-terS-coding SaPIs, SaPIbov5, and found that it uses two different, undescribed packaging strategies. SaPIbov5 is packaged in full-sized phage-like particles either by typical pac-type helper phages, or by cos-type phages—i.e., it has both pac and cos sites and uses the two different phage-coded TerSs. This is an example of SaPI packaging by a cos phage, and in this, it resembles the P4 plasmid of Escherichia coli. Cos-site packaging in Staphylococcus aureus is additionally unique in that it requires the HNH nuclease, carried only by cos phages, in addition to the large terminase subunit, for cos-site cleavage and melting.

Characterization of several of the phage-inducible SaPIs and their helper phages has established that the pac (or headful) mechanism is used for encapsidation. In keeping with this concept, some SaPIs encode a homolog of TerS, which complexes with the phage-coded large terminase subunit TerL to enable packaging of the SaPI DNA in infectious particles composed of phage proteins. These also contain a morphogenesis (cpm) module that causes the formation of small capsids commensurate with the small SaPI genomes. Among the SaPI sequences first characterized, there were several that did not include either a TerS homolog or a cpm homolog, and the same is true of several subsequently identified SaPIs from bovine sources and for many phage-inducible chromosomal islands from other species. It was assumed, for these several islands, either that they were defective derivatives of elements that originally possessed these genes, or that terS and cpm genes were present but not recognized by homology.

Quiles-Puchalt et al observed that an important feature of ϕSLT/SaPIbov5 packaging is the requirement for an HNH nuclease, which is encoded next to the ϕSLT terminase module. Proteins carrying HNH domains are widespread in nature, being present in organisms of all kingdoms. The HNH motif is a degenerate small nucleic acid-binding and cleavage module of about 30-40 aa residues and is bound by a single divalent metal ion. The HNH motif has been found in a variety of enzymes playing important roles in many different cellular processes, including bacterial killing; DNA repair, replication, and recombination; and processes related to RNA. HNH endonucleases are present in a number of cos-site bacteriophages of Gram-positive and -negative bacteria, always adjacent to the genes encoding the terminases and other morphogenetic proteins. Quiles-Puchalt et al have demonstrated that the HNH nucleases encoded by ϕ12 and the closely related ϕSLT have nonspecific nuclease activity and are required for the packaging of these phages and of SaPIbov5. Quiles-Puchalt et al have shown that HNH and TerL are jointly required for cos-site cleavage. Quiles-Puchalt et al have also observed that only cos phages of Gram-negative as well as of Gram-positive bacteria encode HNH nucleases, consistent with a special requirement for cos-site cleavage as opposed to pac-site cleavage, which generates flush-ended products. The demonstration that HNH nuclease activity is required for some but not other cos phages suggests that there is a difference between the TerL proteins of the two types of phages—one able to cut both strands and the other needing a second protein to enable the generation of a double-stranded cut.

The invention, also involves, in certain configurations the use of mobile genetic elements (MGEs). Thus, there are provided the following Clauses. Any of the other configurations, Aspects, Examples or description of the invention above or elsewhere herein are combinable mutatis mutandis with any of these Clauses:—
1. A composition for use in antibacterial treatment of bacteria, the composition comprising an engineered mobile genetic element (MGE) that is capable of being mobilised in a first bacterial host cell of a first species or strain, the cell comprising a first phage genome, wherein in the cell the MGE is mobilised using proteins encoded by the phage and replication of first is inhibited, wherein the MGE encodes an antibacterial agent or encodes a component of such an agent.
   In the alternative, instead of a bacteria, the host cell is a archaeal cell and instead of a phage there is a virus that is capable of infecting the archaeal cell.
   In an example, the MGE is capable of integration into the genome of the host cell comprising the genome of a first phage, for example integration in the chromosome of the host cell and/or an episome thereof.
   Optionally, the MGE inhibits first phage replication.
   In an example, first phage replication is totally inhibited. In an example, it is reduced by at least 50, 60, 70, 80 or 90% compared to replication in the absence of the MGE in host cells. This can be assessed by a standard in vitro plaque assay to determine the relative amount of first phage plaque formation.
   Optionally, in the presence of the agent,
(i) host cells are killed by the antibacterial agent;
(ii) growth or proliferation of host cells is reduced; and/or
(iii) host cells are sensitised to an antibiotic, whereby the antibiotic is toxic to the cells.
2. The composition of Clause 1, wherein the agent is toxic to cells of the same species or strain as the host cell.
3. The composition of Clause 1 or 2, wherein the agent is toxic to cells of a species or strain that is different from the strain or species of the host cell.
4. The composition of Clause 1, wherein the agent is toxic to cells of the same species as the host cell, and wherein the host cell has been engineered so that the agent is not toxic to the host cell.
5. The composition of Clause 4, wherein the agent is a guided nuclease system (optionally a CRISPR/Cas system) and cells of the same species as the host cell comprise a target sequence that is cut by the nuclease, wherein the target sequence has been removed or altered in the host cell whereby the nuclease is not capable of cutting the target sequence.
   Viruses undergo lysogenic and lytic cycles in a host cell. If the lysogenic cycle is adopted, the phage chromosome can be integrated into the bacterial chromosome, or it can establish itself as a stable plasmid in the host, where it can remain dormant for long periods of time. If the lysogen is induced, the phage genome is excised from the bacterial chromosome and initiates the lytic cycle, which culminates in lysis of the cell and the release of phage particles. The lytic cycle leads to the production of new phage particles which are released by lysis of the host.
6. The composition of any preceding Clause, wherein the first phage is a temperate phage.
7. The composition of any preceding Clause, wherein the first cell comprises the first phage as a prophage.
8. The composition of any one of Clauses 1 to 5, wherein the first phage is a lytic phage.
9. The composition of any preceding Clause, wherein in the presence of a first phage the mobilisation of the MGE causes host cell lysis.
10. The composition of any preceding Clause, wherein the MGE is capable of being packaged in transduction particles that comprise some, but not all, structural proteins of the first phage.
   “Transduction particles” may be phage or smaller than phage and are particles that are capable of transducing nucleic acid encoding the antibiotic or component thereof into target bacterial cells.
   Examples of structural proteins are phage proteins selected from one, more or all of the major head and tail proteins, the portal protein, tail fibre proteins, and minor tail proteins.
   The MGE comprises a packaging signal sequence operable with proteins encoded by the first phage to package the MGE (or at least nucleic acid thereof encoding the agent or one or more components thereof) into transduction particles that are capable of infecting host cells of the same species or strain as the first host cell.
11. The composition of any preceding Clause, wherein mobilisation of the MGE comprises packaging of copies of the MGE or nucleic acid encoding the agent or component into transduction particles that are capable of transferring the copies into target bacterial cells for antibacterial treatment of the target cells.
12. The composition of Clause 10 or 11, wherein the transduction particles are particles of second phage that are capable of infecting cells of said first species or strain.
13. The composition of any one of Clauses 10 to 12, wherein the transduction particles are non-self replicative particles.
   A “non-self replicative transduction particle” refers to a particle, (eg, a phage or phage-like particle; or a particle produced from a genomic island (eg, a SaPI) or a modified version thereof) capable of delivering a nucleic acid molecule encoding an antibacterial agent or component into a bacterial cell, but does not package its own replicated genome into the transduction particle. In an alternative herein, instead of a phage, there is used or packaged a virus that infects an animal, human, plant or yeast cell. For example, an adenovirus when the cell is a human cell.
14. The composition of any preceding Clause, wherein the MGE is devoid of genes encoding phage structural proteins.
   Optionally, the MGE is devoid of one or more phage genes rinA, terS and terL.
   In an example, in a host cell a protein complex comprising the small terminase (encoded by terS) and large terminase (encoded by terL) proteins is able to recognise and cleave a double-stranded DNA molecule of the MGE at or near the pac site (cos site or other packaging signal sequence comprised by the MGE), and this allows the MGE or plasmid DNA molecule to be packaged into a phage capsid. When first phage as prophage in the host cell is induced, the lytic cycle of the phage produces the phage's structural proteins and the phage's large terminase protein. The MGE or plasmid is replicated, and the small terminase protein encoded by the MGE or plasmid is expressed. The replicated MGE or plasmid DNA containing the terS (and the nucleotide sequence encoding the antibacterial agent or component) are packaged into phage capsids, resulting in non-self replicative transduction particles carrying only MGE or plasmid DNA.
15. The composition of any one of Clauses 1 to 13, wherein the MGE comprises phage structural genes and a packaging signal sequence and the first phage is devoid of a packaging signal sequence.
16. The composition of any preceding Clause, wherein the MGE is a modified version of a MGE that is naturally found in bacterial cells of the first species or strain.
17. The composition of any preceding Clause, wherein the MGE comprises a modified genomic island.
   Optionally, the genomic island is an island that is naturally found in bacterial cells of the first species or strain. In an example, the genomic island is selected from the group consisting of a SaPI, a SaPI1, a SaPI2, a SaPIbov1 and a SaPibov2 genomic island.
18. The composition of any preceding Clause, wherein the MGE comprises a modified pathogenicity island.
   Optionally, the pathogenicity island is an island that is naturally found in bacterial cells of the first species or strain, eg, a Staphylococcus SaPI or a Vibrio PLE or a P. aeruginosa pathogenicity island (eg, a PAPI or a PAGI, eg, PAPI-1, PAGI-5, PAGI-6, PAGI-7, PAGI-8, PAGI-9, PAGI-10, or PAGI-
19. The composition of Clause 18, wherein the pathogenicity island is a SaPI (S aureus pathogenicity island).
20. The composition of Clause 19, wherein the first phage is ϕ11, 80α, ϕ12 or ϕSLT. Staphylococcus phage 80α appears to mobilise all known SaPIs. Thus, in an example, the MGE comprises a modified SaPI and the first phage is a 80α.
21. The composition of Clause 18, wherein the pathogenicity island is a V. cholerae PLE (phage-inducible chromosomal island-like element) and optionally the first phage is ICP1.
22. The composition of Clause 18, wherein the pathogenicity island is a E coli PLE.
23. The composition of any one of Clauses 1 to 16, wherein the MGE comprises P4 DNA, eg, a P4 packaging signal sequence.
24. The composition of Clause 23, wherein the first phage are P2 phage or a modified P2 phage that is self-replicative defective; optionally present as a prophage.
25. The composition of any preceding Clause, wherein the MGE comprises a pacA gene of the Enterobacteriaceae bacteriophage P1.
26. The composition of any preceding Clause, wherein the MGE comprises a packaging initiation site sequence, optionally a packaging initiation site sequence of P1.
27. The composition of any preceding Clause, wherein the MGE comprises a nucleotide sequence that is beneficial to cells of the first species or strain, optionally encoding a protein that is beneficial to cells of the first species or strain.
   This is useful where, not only does the presence of the MGE reduce first phage replication in the host cell, but also the MGE is taken up and may provide a survival, growth or other benefit to the host cell, promoting uptake and/or retention of MGEs by host cells. In an example, expression of the antibacterial agent in the host cell is under the control of an inducible promoter or weak promoter to allow for a period where uptake of MGEs into host cells may be favoured owing to the presence of the nucleotide sequence that is beneficial to cells of the first species or strain.
28. The composition of any preceding Clause, wherein the MGE is devoid of rinA.
29. The composition of any preceding Clause, wherein the MGE is is devoid of terL.
30. The composition of any preceding Clause, wherein the MGE comprises a terS or a homologue thereof, and optionally is devoid of any other terminase gene.
   The terS homologues are sequences which, like terS, recognise the SaPI-specific pac site (or other packaging sequence) comprised by the MGE or plasmid and determine packaging specificity for packaging the MGE.
   Examples of terminase genes are pacA, pacB, terA, terB and terL.
31. The composition of any preceding Clause, wherein the first phage is a pac-type phage (eg, ϕ11 or 80α) operable with a pac comprised by the MGE.
32. The composition of any one of Clauses 1 to 30, wherein the first phage is a cos-type phage (eg, ϕ12 or ϕSLT) operable with a cos comprised by the MGE.
   Optionally, the phage is P2. Optionally, the first phage is a T7 or T7-like phage that recognises direct repeat sequences comprised by the MGE for packaging.
33. The composition of any preceding Clause, wherein the plasmid or MGE comprises a pac and/or cos sequence or a homologue thereof.
34. The composition of any preceding Clause, wherein the plasmid or MGE comprises a terS or a homologue thereof and optionally devoid of terL.
   The terS homologues are sequences which, like terS, recognise the SaPI-specific pac site (or other packaging sequence) comprised by the MGE or plasmid and determine packaging specificity for packaging the MGE.
   In an example, the terS comprises the sequence of SEQ ID NO: 2:—

SEQ ID NO: 2
AATTGGCAGTAAAGTGGCAGTTTTTGATACCTAAAATGAGATATTATGATAGTGTAGGATAT
TGACTATCTTACTGCGTTTCCCTTATCGCAATTAGGAATAAAGGATCTATGTGGGTTGGCTG
ATTATAGCCAATCCTTTTTTAATTTTAAAAAGCGTATAGCGCGAGAGTTGGTGGTAAATGAA
ATGAACGAAAAACAAAAGAGATTCGCAGATGAATATATAATGAATGGATGTAATGGTAAAAA
AGCAGCAATTTCAGCAGGTTATAGTAAGAAAACAGCAGAGTCTTTAGCAAGTCGATTGTTAA
GAAATGTTAATGITTCGGAATATATTAAAGAACGATTAGAACAGATACAAGAAGAGCGTTTA
ATGAGCATTACAGAAGCTTTAGCGTTATCTGCTTCTATTGCTAGAGGAGAACCTCAAGAGGC
TTACAGTAAGAAATATGACCATTTAAACGATGAAGTGGAAAAAGAGGTTACTTACACAATCA
CACCAACTTTTGAAGAGCGICAGAGATCTATTGACCACATACTAAAAGTTCATGGTGCGTAT
ATCGACAAAAAAGAAATTACTCAGAAGAATATTGAGATTAATATTGGTGAGTACGATGACGA
AAGTTAAATTAAACTTTAACAAACCATCTAATGTTTTCAACAG

35. The composition of Clause 34, wherein the terS is a S aureus bacteriophage φ80α terS or a bacteriophage φ11 terS.
36. The composition of any preceding Clause, wherein the MGE is a modified SaPIbov1 or SaPIbov5 and is devoid of a terS.
37. The composition of any preceding Clause, wherein the first phage is devoid of a functional packaging signal sequence and the MGE comprises a packaging signal sequence operable with proteins encoded by the first phage for producing transduction particles that package copies of the MGE or copies of a nucleic acid encoding the agent or component.
38. The composition of any preceding Clause, wherein the MGE or plasmid comprises a Ppi or homologue, which is capable of complexing with first phage TerS, thereby blocking function of the TerS.
39. The composition of any preceding Clause, wherein the MGE comprises a morphogenesis (cpm) module.
40. The composition of any preceding Clause, wherein the MGE comprises cpmA and/or cpmB.
   Optionally the cpmA and B are from any SaPI disclosed herein. In an example any SaPI is a SaPI disclosed in FIG. 3 and optionally the host cell or target cell is any corresponding Staphylococcus disclosed in the table.
41. The composition of any preceding Clause, wherein the MGE or first phage comprises one, more or all genes cp1, cp2, and cp3.
   In an example, the MGE comprises a modified SaPI and comprises one, more or all genes cp1, cp2, and cp3.
42. The composition of any preceding Clause, wherein the MGE or first phage encodes a HNH nuclease.
43. The composition of any preceding Clause, wherein the MGE or first phage comprises an integrase gene that encodes an integrase for excising the MGE and integrating the MGE into a bacterial cell genome.
44. The composition of any preceding Clause, wherein the MGE is devoid of a functional integrase gene, and the first phage or host cell genome (eg, bacterial chromosome or a bacterial episome) comprises a functional integrase gene.
45. The composition of any preceding Clause, wherein the transcription of MGE nucleic acid is under the control of a constitutive promoter, for transcription of copies of the agent or component in a host cell.
   Optionally, Constitutive transcription and production of the agent in target cells may be used where the target cells should be killed, eg, in medical settings.
   Optionally, the transcription of MGE nucleic acid is under the control of an inducible promoter, for transcription of copies of the agent or component in a host cell. This may be useful, for example, to control switching on of the antibacterial activity against target bacterial cells, such as in an environment (eg, soil or water) or in an industrial culture or fermentation container containing the target cells. For example, the target cells may be useful in an industrial process (eg, for fermentation, eg, in the brewing or dairy industry) and the induction enables the process to be controlled (eg, stopped or reduced) by using the antibacterial agent against the target bacteria.
46. The composition of Clause 45, wherein the promoter is foreign to the host cell.
47. The composition of Clause 45 or 46, wherein the promoter comprises a nucleotide sequence that is at least 80% identical to an endogenous promoter sequence of the host cell.
48. The composition of any preceding Clause comprising a nucleic acid that is separate from the MGE, wherein the nucleic acid comprises all genes necessary for producing first phage particles.
49. The composition of any one of Clauses 1 to 47 comprising a nucleic acid that is separate from the MGE, wherein the nucleic acid comprises less than, all genes necessary for producing first phage particles, but comprises genes encoding structural proteins for production of transduction particles that package MGE nucleic acid encoding the antibacterial agent or one or more components thereof.
   When the agent comprises a plurality of components, eg, wherein the agent is a CRISPR/Cas system, or is a CRISPR array encoding crRNA or a nucleic acid encoding a guide RNA (eg, single guide RNA) operable with a Cas in host cells, wherein the crRNA or gRNA guides the Cas to a target sequence in the host cell to modify the target (eg, cut it or repress transcription from it).
50. The composition of Clause 48 or 49, wherein the genes are comprised by the host cell chromosome and/or one or more host cell episome(s).
51. The composition of Clause 50, wherein the genes are comprised by a chromosomally-integrated prophage of the first phage.
52. The composition of any preceding Clause, wherein the agent is a guided nuclease system or a component thereof, wherein the agent is capable of recognising and cutting host cell DNA (eg, chromosomal DNA).
   In examples, such cutting causes one or more of the following:—
(i) The host cell is killed by the antibacterial agent;
(ii) growth or proliferation of the host cell is reduced; and/or
(iii) The host cell is sensitised to an antibiotic, whereby the antibiotic is toxic to the cell.
53. The composition of Clause 52, wherein the guided nuclease system is selected from a CRISPR/Cas system, TALEN system, meganuclease system or zinc finger system.
54. The composition of Clause 52, wherein the system is a CRISPR/Cas system and each MGE encodes a (a) CRISPR array encoding crRNA or (b) a nucleic acid encoding a guide RNA (gRNA, eg, single guide RNA), wherein the crRNA or gRNA is operable with a Cas in target bacterial cells, wherein the crRNA or gRNA guides the Cas to a target nucleic acid sequence in the host cell to modify the target sequence (eg, cut it or repress transcription from it).
   Optionally, the Cas is a Cas encoded by a functional endogenous nucleic acid of a host cell. For example, the target is comprised by a DNA or RNA of the host cell.
55. The composition of Clause 52, wherein the system is a CRISPR/Cas system and each MGE encodes a Cas (eg, a Cas nuclease) that is operable in a target bacterial cells to modify a target nucleic acid sequence comprised by the target cell.
56. The composition of Clause 53, 54 or 55, wherein the Cas is a Cas3, Cas9, Cas13, CasX, CasY or Cpf1.
57. The composition of any one of Clauses 52 to 56, wherein the system is a CRISPR/Cas system and each MGE encodes one or more Cascade Cas (eg, Cas, A, B, C, D and E).
58. The composition of any one of Clauses 52 to 57, wherein each MGE further encodes a Cas3 that is operable in a target bacterial cell with the Cascade Cas.
59. The composition of any preceding Clause, wherein the first species or strain is a gram positive species or strain.
60. The composition of any one of Clauses 1 to 58, wherein the first species or strain is a gram negative species or strain.
61. The composition of any preceding Clause, wherein the first species or strain is selected from Table 1.
   In an example, the first species of strain is a Staphylococcus (eg, S aureus) species or strain and optionally the MGE is a modified SaPI; and optionally the first phage is a φ80α or φ11. In an example, the first species of strain is a Vibrio (eg, V cholerae) species or strain and optionally the MGE is Vibrio (eg, V cholerae) PLE.
62. The composition of any preceding Clause, wherein the first species or strain is selected from Shigella, E coli, Salmonella, Serratia, Klebsiella, Yersinia, Pseudomonas and Enterobacter.
   These are species that P2 phage can infect. Thus, in an embodiment, the MGE comprises one or more P4 sequences (eg, a P4 packaging sequence) and the first phage is P2. Thus, the MGE is packaged by P2 structural proteins and the resultant transduction particles can infect a broad spectrum of species, ie, two or more of Shigella, E coli, Salmonella, Serratia, Klebsiella, Yersinia, Pseudomonas and Enterobacter.
63. A nucleic acid vector comprising a MGE integrated therein, wherein the MGE is according to any preceding Clause and the vector is capable of transferring the MGE or a copy thereof into a host bacterial cell.
   Suitable vectors are plasmids (eg, conjugative plasmids) or viruses (eg, phage or packaged phagemids).
64. The vector of Clause 63, wherein the vector is a shuttle vector.
   A shuttle vector is a vector (usually a plasmid) constructed so that it can propagate in two different host species. Therefore, DNA inserted into a shuttle vector can be tested or manipulated in two different cell types.
65. The vector of Clause 63, wherein the vector is a plasmid, wherein the plasmid is capable of being transformed into a host bacterial cell comprising a first phage.
66. A non-self replicative transduction particle comprising said MGE or vector of any preceding Clause.
   By “non-replicative” it is meant that the MGE is not capable by itself of self-replicating. For example, the MGE is devoid of one or more nucleotide sequences encoding a protein (eg, a structural protein) that is necessary to produce a transduction particle comprising a copy of the MGE.
67. A composition comprising a plurality of transduction particles, wherein each particle comprises a MGE or vector according to any one of Clauses 1 to 65, wherein the transduction particles are capable of transferring the MGEs, or nucleic acid encoding the agent or component, or copies thereof into target bacterial cells, wherein
   a. target cells are killed by the antibacterial agent;
   b. growth or proliferation of target cells is reduced; or
   c. target cells are sensitised to an antibiotic, whereby the antibiotic is toxic to the cells.
      In an example, the reduction in growth or proliferation of host cells is at least 50, 60, 70, 80, 90 or 95%. The antibiotic can be any antibiotic disclosed herein.
68. The composition of Clause 67, wherein the agent is a guided nuclease system or a component thereof, wherein the agent is capable of recognising and cutting host cell DNA (eg, chromosomal DNA) whereby
   a. target cells are killed by the antibacterial agent;
   b. growth or proliferation of target cells is reduced; or
   c. target cells are sensitised to an antibiotic, whereby the antibiotic is toxic to the cells.
69. A composition comprising a plurality of non-self replicative transduction particles, wherein each particle comprises a MGE or plasmid according to any one of Clauses 1 to 65, wherein the transduction particles are capable of transferring the MGEs, or nucleic acid encoding the agent or component, or copies thereof into target bacterial cells, wherein the agent is a CRISPR/Cas system and the component comprises a nucleic acid encoding a crRNA or a guide RNA that is operable with a Cas in a target bacterial cell to guide the Cas to a target nucleic acid sequence of the cell to modify the sequence, whereby
   a. target cells are killed by the antibacterial agent;
   b. growth or proliferation of target cells is reduced; or
   c. target cells are sensitised to an antibiotic, whereby the antibiotic is toxic to the cells.
      In an example, the reduction in growth or proliferation of host cells is at least 50, 60, 70, 80, 90 or 95%. The antibiotic can be any antibiotic disclosed herein.
70. A kit comprising the composition of Clause 69 and said antibiotic.
71. The composition of Clause 69, wherein the composition comprises said antibiotic.
72. The composition of any one of Clauses 67 to 69, wherein less than 10% of transduction particles comprise by the composition are first phage particles.
73. The composition of any one of Clauses 67 to 69, wherein no first phage particles are present in the composition.
74. The MGE, vector, particle, composition or kit of any preceding Clause for medical use in a human or animal patient.
75. The MGE, vector, particle, composition or kit of any preceding Clause for treating or preventing an infection by target bacterial cells in a human or animal patient, wherein the antibacterial agent is toxic to the target cells.
76. The MGE, vector, particle, composition or kit of any preceding Clause for treating or preventing an infection by target bacterial cells in a human or animal patient, wherein in the presence of the antibacterial agent
   a. target cells are killed by the antibacterial agent;
   b. growth or proliferation of target cells is reduced; and/or
   c. target cells are sensitised to an antibiotic, whereby the antibiotic is toxic to the cells.
77. A method of producing a plurality of transduction particles, the method comprising combining the composition of any one of Clauses 1 to 62, 67 to 69 and 71 to 76 with host bacterial cells of said first species, wherein the cells comprise the first phage, allowing a plurality of said MGEs to be introduced into host cells and culturing the host cells under conditions in which first phage-encoded proteins are expressed and MGE copies are packaged by first phage proteins to produce a plurality of transduction particles, and optionally separating the transduction particles from cells and obtaining a plurality of transduction particles separated from cells.
78. The method of Clause 77, comprising separating the transduction particles from any first phage, optionally by filtering or centrifugation, thereby obtaining a plurality of transduction particles in the absence of first phage.
79. The method of Clause 77 or 78, wherein the particles encode a guided nuclease system (optionally a CRISPR/Cas system) or component thereof for cutting a target nucleic acid sequence comprised by target bacterial cells.
80. The method of Clause 79, wherein the sequence is comprised by an antibiotic resistance gene and the method comprises combining the plurality of particles with said antibiotic in a kit or a mixture.
81. The method of any one of Clauses 77 to 80, wherein said conditions comprise induction of a lytic cycle of the first phage.
82. A bacterial host cell comprising a first phage and a MGE, vector or particle as recited in any one of Clauses 1 to 66, wherein the agent is toxic to cells of the same species as the host cell, and wherein the host cell has been engineered so that the agent is not toxic to the host cell.
83. A bacterial host cell comprising a first phage, wherein the cell is comprised by a kit, the kit further comprising a composition as recited in any one of Clauses 1 to 62, 67 to 69 and 71 to 76, wherein the agent is toxic to cells of the same species as the host cell, and wherein the host cell has been engineered so that the agent is not toxic to the host cell.
84. The cell of Clause 83, wherein the agent is a guided nuclease system (optionally a CRISPR/Cas system) and cells of the same species as the host cell comprise a target sequence that is cut by the nuclease, wherein the target sequence has been removed or altered in the host cell whereby the nuclease is not capable of cutting the target sequence.
85. A bacterial host cell comprising a first phage and a MGE, vector or particle as recited in any one of Clauses 1 to 66, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.
86. A bacterial host cell comprising a first phage, wherein the cell is comprised by a kit, the kit further comprising a composition as recited in any one of Clauses 1 to 62, 67 to 69 and 71 to 76, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.
87. The cell of Clause 86, wherein the first phage is a prophage.
88. A bacterial host cell comprising a MGE, vector or particle as recited in any one of Clauses 1 to 66 and nucleic acid under the control of one or more inducible promoters, wherein the nucleic acid encodes all structural proteins necessary to produce a transduction particle that packages a copy of the MGE or plasmid, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.
89. The cell of Clause 88, wherein the structural proteins are structural proteins of a lytic phage.
90. The cell of Clause 88 or 89, wherein the nucleic acid comprises terS and/or terL.
91. The cell of any one of Clauses 88 to 90, wherein the host and second cells are of the same species and the host cell has been engineered so that the antibiotic is not toxic to the host cell.
92. The cell of any one of Clauses 88 to 91, wherein the nucleic acid is comprised by a plasmid.
93. The cell of any one of Clauses 88 to 92, wherein the agent is a guided nuclease system (optionally a CRISPR/Cas system) and the second cells comprise a target sequence that is cut by the nuclease, wherein the target sequence is absent in the genome of the host cell whereby the nuclease is not capable of cutting the host cell genome.
94. The composition, vector, particle, kit or method of any preceding Clause, wherein the cell, host cell or target cell is selected from a Staphylococcal, Vibrio, Pseudomonas, Clostridium, E coli, Helicobacter, Klebsiella and Salmonella cell.
95. A plasmid comprising
   a. A nucleotide sequence encoding an antibacterial agent or component thereof for expression in target bacterial cells;
   b. A constitutive promoter for controlling the expression of the agent or component;
   c. An optional terS nucleotide sequence;
   d. An origin of replication (ori); and
   e. A phage packaging sequence (optionally pac, cos or a homologue thereof); and
   f. the plasmid being devoid of
   g. All nucleotide sequences encoding phage structural proteins necessary for the production of a transduction particle (optionally a phage), or the plasmid being devoid of at least one of such sequences; and
   h. Optionally terL.
96. The plasmid of Clause 95, wherein the antibacterial agent is a CRISPR/Cas system and the plasmid encodes a crRNa or guide RNA (eg, single gRNA) that is operable with a Cas in the target cells to guide the Cas to a target nucleotide sequence to modify (eg, cut) the sequence, whereby
   a. target cells are killed by the antibacterial agent;
   b. growth or proliferation of target cells is reduced; or
   c. target cells are sensitised to an antibiotic, whereby the antibiotic is toxic to the cells.
97. The plasmid of Clause 95 or 96, wherein the antibacterial agent is a CRISPR/Cas system and the plasmid encodes a Cas that is operable with a crRNa or guide RNA (eg, single gRNA) in the target cells to guide the Cas to a target nucleotide sequence to modify (eg, cut) the sequence, whereby
   a. target cells are killed by the antibacterial agent;
   b. growth or proliferation of target cells is reduced; or
   c. target cells are sensitised to an antibiotic, whereby the antibiotic is toxic to the cells.
98. The plasmid of Clause 97, wherein the plasmid further encodes said crRNA or gRNA.
99. A host cell comprising the plasmid of any one of Clauses 95 to 98, wherein the host cell does not comprise the target nucleotide sequence.
100. The host cell of Clause 99, wherein the cell is capable of replicating the plasmid and packaging the replicated plasmid in transduction particles that are capable of infecting target bacterial cells.
101. The host cell of Clause 99 or 100, wherein the host cell comprises, integrated in the cell chromosome and/or one or more episomes of the cell,
   a. A terL;
   b. An optional terS; and
   c. Expressible nucleotide sequences encoding all structural proteins necessary for the production of transduction particles that package copies of the plasmid;
   d. wherein the chromosome and episomes of the cell (other than said plasmid) are devoid of a phage packaging sequence, wherein the phage packaging sequence comprised by the plasmid is operable together with the product of said terS and terL in the production of packaged plasmid.
102. The cell of Clause 101, wherein the terL, optional terS and nucleotide sequences encoding the structural proteins are comprised by a phage (optionally a prophage) genome in the host cell.
103. A bacterial host cell comprising the genome of a helper phage that is incapable of self-replication, optionally wherein the genome is present as a prophage, and a plasmid according to any one of Clauses 95 to 98, wherein the helper phage is operable to package copies of the plasmid in transduction particles, wherein the particles are capable of infecting bacterial target cells to which the antibacterial agent is toxic.
104. The cell of Clause 103, wherein the host cell is a cell of first species or strain and the target cells are of the same species or strain, and optionally wherein the hosts cell is an engineered cell that to which the antibacterial agent is not toxic.
105. The cell of Clause 103, wherein the host cell is a cell of first species or strain and the target cells are of a different species or strain, wherein the antibacterial agent is not toxic to the host cell.
106. A method of making a plurality of transduction particles, the method comprising culturing a plurality of host cells according to any one of Clauses 103 to 105, optionally inducing a lytic cycle of the helper phage, and incubating the cells under conditions wherein transducing particles comprising packaged copies of the plasmid are created, and optionally separating the particles from the cells to obtain a plurality of transduction particles.
107. A plurality of transduction particles obtainable by the method of Clause 106 for use in medicine, eg, for treating or preventing an infection of a human or animal subject by target bacterial cells, wherein transducing particles are administered to the subject for infecting target cells and killing the cells using the antibacterial agent.
108. A method of making a plurality of transduction particles, the method comprising
   i. Producing host cells whose genomes comprise nucleic acid encoding structural proteins necessary to produce transduction particles that can package first DNA, wherein the genomes are devoid of a phage packaging signal, wherein the expression of the proteins is under the control of inducible promoter(s);
   ii. Producing first DNA encoding an antibacterial agent or a component thereof (eg, as defined in any preceding Clause), wherein the DNA comprises a phage packaging signal;
   iii. Introducing the DNA into the host cells;
   iv. Inducing production of the structural proteins in host cells, whereby transduction particles are produced that package the DNA;
   v. Optionally isolating a plurality of the transduction particles; and
   vi. Optionally formulating the particles into a pharmaceutical composition for administration to a human or animal for medical use.
109. The method of Clause 108, wherein the DNA comprises a MGE as defined in any preceding Clause.
110. The method of Clause 108 or 109, wherein the structural proteins are P2 phage proteins and optionally the packaging signal is a P4 phage packaging signal.
111. The method of Clause 108 or 109, wherein the DNA comprises a modified SaPI or a genomic island DNA.
112. The method of any one of Clauses 108 to 111, wherein the cells in step (iv) comprise a gene encoding a helper phage activator, optionally wherein the activator is a P4 phage delta or ogr protein when the structural proteins are P2 proteins; or the activator is a SaPI rinA, ptiA, ptiB or ptiM when the MGE comprises a modified SaPI; and optionally the expression of the activator(s) is controlled by an inducible promoter, eg, a T7 promoter.
113. The method of any one of Clauses 108 to 112, wherein the packaging signal is P4 phage Sid and/or psu; or the signal is SaPI cpmA and/or cpmB.
   This is useful for packaging DNAs into smaller capsids.
114. The method of any one of Clauses 108 to 113, wherein the cell genomes comprise prophages, wherein each prophage comprises said nucleic acid encoding structural proteins.
115. The method of Clause 114, wherein the prophages are P2 prophages devoid of cos and optionally one, more or all genes selected from int, cox orf78, B, orf80, orf81, orf82, orf83, A, orf91, tin, old, orf30 and fun(Z); and optionally the packaging signal of (ii) is a cos or P4 packaging signal.
116. The method of Clause 114 or 115, wherein the prophages are P2 prophages devoid of cos and comprising genes from Q to S, V to G and F_{I} to ogr.
117. The method of Clause 114, wherein the prophages are phi11 prophages devoid of a packaging signal and comprising gene 29 (terS) to gene 53 (lysin); and optionally the packaging signal of (ii) is a phi11 packaging signal.
118. A plurality of transduction particles obtainable by the method of any one of Clauses 108 to 117.
119. The particles of Clause 118 for administration to a human or animal for medical use.

### Further Concepts of the Invention are as Follows:—

The present invention is optionally for an industrial or domestic use, or is used in a method for such use. For example, it is for or used in agriculture, oil or petroleum industry, food or drink industry, clothing industry, packaging industry, electronics industry, computer industry, environmental industry, chemical industry, aerospace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

The present invention is optionally for use in an industry or the environment is an industrial environment, wherein the industry is an industry of a field selected from the group consisting of the medical and healthcare; pharmaceutical; human food; animal food; plant fertilizers; beverage; dairy; meat processing; agriculture; livestock farming; poultry farming; fish and shellfish farming; veterinary; oil; gas; petrochemical; water treatment; sewage treatment; packaging; electronics and computer; personal healthcare and toiletries; cosmetics; dental; non-medical dental; ophthalmic; non-medical ophthalmic; mineral mining and processing; metals mining and processing; quarrying; aviation; automotive; rail; shipping; space; environmental; soil treatment; pulp and paper; clothing manufacture; dyes; printing; adhesives; air treatment; solvents; biodefence; vitamin supplements; cold storage; fibre retting and production; biotechnology; chemical; industrial cleaning products; domestic cleaning products; soaps and detergents; consumer products; forestry; fishing; leisure; recycling; plastics; hide, leather and suede; waste management; funeral and undertaking; fuel; building; energy; steel; and tobacco industry fields.

In an example, the first DNA, first phage or vector comprises a CRISPR array that targets target bacteria, wherein the array comprises one, or two or more spacers (eg, 2, 3, 4, 5, 6, 7, 8, 9, 10, 20, 30, 40, 50 or more spacers) for targeting the genome of target bacteria.

In an example, the target bacteria are comprised by an environment as follows. In an example, the environment is a microbiome of a human, eg, the oral cavity microbiome or gut microbiome or the bloodstream. In an example, the environment is not an environment in or on a human. In an example, the environment is not an environment in or on a non-human animal. In an embodiment, the environment is an air environment. In an embodiment, the environment is an agricultural environment. In an embodiment, the environment is an oil or petroleum recovery environment, eg, an oil or petroleum field or well. In an example, the environment is an environment in or on a foodstuff or beverage for human or non-human animal consumption.

In an example, the environment is a a human or animal microbiome (eg, gut, vaginal, scalp, armpit, skin or oral cavity microbiome). In an example, the target bacteria are comprised by a human or animal microbiome (eg, gut, vaginal, scalp, armpit, skin or oral cavity microbiome).

In an example, the DNAs, phage or composition of the invention are administered intranasally, topically or orally to a human or non-human animal, or is for such administration. The skilled person aiming to treat a microbiome of the human or animal will be able to determine the best route of administration, depending upon the microbiome of interest. For example, when the microbiome is a gut microbiome, administration can be intranasally or orally. When the microbiome is a scalp or armpit microbiome, administration can be topically. When the microbiome is in the mouth or throat, the administration can be orally.

In an example, the environment is harboured by a beverage or water (eg, a waterway or drinking water for human consumption) or soil. The water is optionally in a heating, cooling or industrial system, or in a drinking water storage container.

In an example, the host and/or target bacteria are Firmicutes selected from Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter and Weissella.

In an example, the kit, DNA(s), first phage, helper phage, composition, use or method is for reducing pathogenic infections or for re-balancing gut or oral microbiota eg, for treating or preventing obesity or disease in a human or animal. For example, the first phage, helper phage, composition, use or method is for knocking-down Clostridium difficile or E coli bacteria in a gut microbiota of a human or animal.

In an example, the packaging signal, NPF and/or HPF consists or comprises SEQ ID NO: 1 or a structural or functional homologue thereof.

In an example, the packaging signal, NPF and/or HPF consists or comprises SEQ ID NO: 1 or a nucleotide sequence that is at least 70, 75, 80, 85, 90, 91, 92, 93, 94, 95, 96, 97, 98 or 99% identical thereto.

In an example, the disease or condition is a cancer, inflammatory or autoimmune disease or condition, eg, obesity, diabetes IBD, a GI tract condition or an oral cavity condition.

Optionally, the environment is comprised by, or the target bacteria are comprised by, a gut microbiota, skin microbiota, oral cavity microbiota, throat microbiota, hair microbiota, armpit microbiota, vaginal microbiota, rectal microbiota, anal microbiota, ocular microbiota, nasal microbiota, tongue microbiota, lung microbiota, liver microbiota, kidney microbiota, genital microbiota, penile microbiota, scrotal microbiota, mammary gland microbiota, ear microbiota, urethra microbiota, labial microbiota, organ microbiota or dental microbiota. Optionally, the environment is comprised by, or the target bacteria are comprised by, a plant (eg, a tobacco, crop plant, fruit plant, vegetable plant or tobacco, eg on the surface of a plant or contained in a plant) or by an environment (eg, soil or water or a waterway or aqueous liquid).

Optionally, the disease or condition of a human or animal subject is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(lll) Sinusitis;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

Neurodegenerative or CNS Diseases or Conditions for Treatment or Prevention by the Invention

In an example, the neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease, Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and/or reduces the progression of nerve fibre damage. In an example, the method restores nerve myelin and/or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatment and/or prevention of CNS and neurodegenerative diseases and conditions, eg, agents such as immune checkpoint inhibitors, eg, anti-PD-1, anti-PD-L1, anti-TIM3 or other antibodies disclosed therein).

Cancers for Treatment or Prevention by the Method

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyelocytic, myelomonocytic, monocytic and erythroleukemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyngioma, ependymoma, pinealoma, hemangioblastoma, acoustic neuroma, oligodendroglioma, menangioma, neuroblastoma, retinoblastoma and brain metastases).

Autoimmune Diseases for Treatment or Prevention by the Method
1. Acute Disseminated Encephalomyelitis (ADEM)
2. Acute necrotizing hemorrhagic leukoencephalitis
3. Addison's disease
4. Agammaglobulinemia
5. Alopecia areata
6. Amyloidosis
7. Ankylosing spondylitis
8. Anti-GBM/Anti-TBM nephritis
9. Antiphospholipid syndrome (APS)
10. Autoimmune angioedema
11. Autoimmune aplastic anemia
12. Autoimmune dysautonomia
13. Autoimmune hepatitis
14. Autoimmune hyperlipidemia
15. Autoimmune immunodeficiency
16. Autoimmune inner ear disease (AIED)
17. Autoimmune myocarditis
18. Autoimmune oophoritis
19. Autoimmune pancreatitis
20. Autoimmune retinopathy
21. Autoimmune thrombocytopenic purpura (ATP)
22. Autoimmune thyroid disease
23. Autoimmune urticaria
24. Axonal & neuronal neuropathies
25. Balo disease
26. Behcet's disease
27. Bullous pemphigoid
28. Cardiomyopathy
29. Castleman disease
30. Celiac disease
31. Chagas disease
32. Chronic fatigue syndrome
33. Chronic inflammatory demyelinating polyneuropathy (CIDP)
34. Chronic recurrent multifocal osteomyelitis (CRMO)
35. Churg-Strauss syndrome
36. Cicatricial pemphigoid/benign mucosal pemphigoid
37. Crohn's disease
38. Cogans syndrome
39. Cold agglutinin disease
40. Congenital heart block
41. Coxsackie myocarditis
42. CREST disease
43. Essential mixed cryoglobulinemia
44. Demyelinating neuropathies
45. Dermatitis herpetiformis
46. Dermatomyositis
47. Devic's disease (neuromyelitis optica)
48. Discoid lupus
49. Dressler's syndrome
50. Endometriosis
51. Eosinophilic esophagitis
52. Eosinophilic fasciitis
53. Erythema nodosum
54. Experimental allergic encephalomyelitis
55. Evans syndrome
56. Fibromyalgia
57. Fibrosing alveolitis
58. Giant cell arteritis (temporal arteritis)
59. Giant cell myocarditis
60. Glomerulonephritis
61. Goodpasture's syndrome
62. Granulomatosis with Polyangiitis (GPA) (formerly called Wegener's Granulomatosis)
63. Graves' disease
64. Guillain-Barre syndrome
65. Hashimoto's encephalitis
66. Hashimoto's thyroiditis
67. Hemolytic anemia
68. Henoch-Schonlein purpura
69. Herpes gestationis
70. Hypogammaglobulinemia
71. Idiopathic thrombocytopenic purpura (ITP)
72. IgA nephropathy
73. IgG4-related sclerosing disease
74. Immunoregulatory lipoproteins
75. Inclusion body myositis
76. Interstitial cystitis
77. Juvenile arthritis
78. Juvenile diabetes (Type 1 diabetes)
79. Juvenile myositis
80. Kawasaki syndrome
81. Lambert-Eaton syndrome
82. Leukocytoclastic vasculitis
83. Lichen planus
84. Lichen sclerosus
85. Ligneous conjunctivitis
86. Linear IgA disease (LAD)
87. Lupus (SLE)
88. Lyme disease, chronic
89. Meniere's disease
90. Microscopic polyangiitis
91. Mixed connective tissue disease (MCTD)
92. Mooren's ulcer
93. Mucha-Habermann disease
94. Multiple sclerosis
95. Myasthenia gravis
96. Myositis
97. Narcolepsy
98. Neuromyelitis optica (Devic's)
99. Neutropenia
100. Ocular cicatricial pemphigoid
101. Optic neuritis
102. Palindromic rheumatism
103. PANDAS (Pediatric Autoimmune Neuropsychiatric Disorders Associated with Streptococcus)
104. Paraneoplastic cerebellar degeneration
105. Paroxysmal nocturnal hemoglobinuria (PNH)
106. Parry Romberg syndrome
107. Parsonnage-Turner syndrome
108. Pars planitis (peripheral uveitis)
109. Pemphigus
110. Peripheral neuropathy
111. Perivenous encephalomyelitis
112. Pernicious anemia
113. POEMS syndrome
114. Polyarteritis nodosa
115. Type I, II, & III autoimmune polyglandular syndromes
116. Polymyalgia rheumatica
117. Polymyositis
118. Postmyocardial infarction syndrome
119. Postpericardiotomy syndrome
120. Progesterone dermatitis
121. Primary biliary cirrhosis
122. Primary sclerosing cholangitis
123. Psoriasis
124. Psoriatic arthritis
125. Idiopathic pulmonary fibrosis
126. Pyoderma gangrenosum
127. Pure red cell aplasia
128. Raynauds phenomenon
129. Reactive Arthritis
130. Reflex sympathetic dystrophy
131. Reiter's syndrome
132. Relapsing polychondritis
133. Restless legs syndrome
134. Retroperitoneal fibrosis
135. Rheumatic fever
136. Rheumatoid arthritis
137. Sarcoidosis
138. Schmidt syndrome
139. Scleritis
140. Scleroderma
141. Sjogren's syndrome
142. Sperm & testicular autoimmunity
143. Stiff person syndrome
144. Subacute bacterial endocarditis (SBE)
145. Susac's syndrome
146. Sympathetic ophthalmia
147. Takayasu's arteritis
148. Temporal arteritis/Giant cell arteritis
149. Thrombocytopenic purpura (TTP)
150. Tolosa-Hunt syndrome
151. Transverse myelitis
152. Type 1 diabetes
153. Ulcerative colitis
154. Undifferentiated connective tissue disease (UCTD)
155. Uveitis
156. Vasculitis
157. Vesiculobullous dermatosis
158. Vitiligo
159. Wegener's granulomatosis (now termed Granulomatosis with Polyangiitis (GPA).

Inflammatory Diseases for Treatment or Prevention by the Method
1. Alzheimer
2. ankylosing spondylitis
3. arthritis (osteoarthritis, rheumatoid arthritis (RA), psoriatic arthritis)
4. asthma
5. atherosclerosis
6. Crohn's disease
7. colitis
8. dermatitis
9. diverticulitis
10. fibromyalgia
11. hepatitis
12. irritable bowel syndrome (IBS)
13. systemic lupus erythematous (SLE)
14. nephritis
15. Parkinson's disease
16. ulcerative colitis.

### EXAMPLES

### Example 1: Efficient Phage CRISPR Delivery Vehicle Production

Background

We designed a strategy for efficient production of phage particles comprising components of a CRISPR/Cas system for killing target E coli Nissle strain bacteria. So our phage composition will consist of a lysate primarily containing CRISPR/Cas system components packaged in phage particles which will be devoid of phage protein-encoding sequences and which will have no or a very low proportion of helper phage. Also the strategy will work alternatively in less well characterised phage/bacterial strain combinations.

Outline of Strategy for CRISPR/Cas Component Packaging in Hitherto Unknown Phages
(i) Identify a high copy number cloning/shuttle vector (capable of cloning and propagation in a first E coli strain and then transfer to a second bacterial host strain of interest) containing an E coli on for replication in the E coli cloning strain;
(j) Isolate temperate phage against the host (second) bacterium;
(k) Identify or engineer a phage production strain of the host bacteria that has an inactive CRISPR/Cas system (eg, a repressed Cas3 or other nuclease) and which can be infected and lysogenized with the temperate phage; or repress or inactivate the system in the production strain;
(l) In that strain make a lysogen using the temperate phage (helper phage) and test that it can be induced;
(m) Identify the packaging sequence (pac or cos) using PhageTerm (world wide web.ncbi.nlm.nih.gov/pmc/articles/PMC5557969) on whole genome sequenced phage;
(n) Delete the pac/cos packaging signal sequence in the helper phage in the host bacteria;
(o) Incorporate the packaging signal in the shuttle vector along with a CRISPR-array (and other components of the CRISPR/Cas system, such as a Cas9-encoding nucleotide sequence, or Cas3 and/or Cascade-encoding sequence);
(p) Transform the vector into production host strain;
(q) UV or mitomycin C induce and harvest phage comprising the CRISPR/Cas component(s). Alternatively, use a system with inducible RecA in trans to simulate SOS (needs to be activated RecA).

Example of the Above Specifically for E coli Nissle Using Phage P2:

Nissle is useful due to its GRAS (Generally Regarded as Safe) status and P2 has a relatively broad host range (most E coli, Shigella, Klebsiella, Salmonella in addition to DNA delivery into e.g. Pseudomonas; Kahn et al 1991).

We will use pUC19 or other high copy number cloning vector. Temperate phage P2 can lysogenize Nissle. Most E coli K strains have an inactive CRISPR/Cas system and can be infected by P2 and thus all regular cloning hosts can be used (here exemplified by E coli TOP10).

P2 is introduced into TOP10 to produce a lysogen. P2 cannot be induced with mitomycin C or UV but we will use the epsilon anti-repressor from the parasite phage P4 that derepresses P2 and makes it go into lytic phase. We will express this gene from an inducible promoter in the production host strain.

The 325 bp packaging signal sequence as follows will be used

(SEQ ID NO: 1)
GCATGCGTTTTCCTGCCTCATTTTCTGCAAACCGCGCCATTCCCGGCGC
GGTCTGAGCGTGTCAGTGCAACTGCATTAAAACCGCCCCGCAAAGCGGG
CGGGCGAGGCGGGGAAAGCACCGCGCGCAAACCCAGAAGTTAGTTAATT
ATTTGTGTAGTCAAAGTGCCTTGACTACATACCTCGTTAATACATTGGA
GCATAATGAAGAAAATCTATGGCCTATGGTCCAAAACTGTCTTTTTTGA
TGGCACTATCCTGAAAAATATGCAAAAAATAGATTGATGTAAGGTGGTT
CTTGTCAGTGTCGCAAGATCCTTAAGAATTC

The packaging sequence will be deleted in the P2 prophage of the lysogenic production TOP10 strain.

A pUC19 shuttle vector encoding a guide RNA that targets the genome of the target Nissle strain (or alternatively comprising a CRISPR array for producing such a guide RNA) will be constructed and the packaging signal will be added. If the target Nissle harbours it own endogenous CRISPR/Cas system, we will use an activation strategy to activate the endogenous Cas3 by including Cas activating genes in the vector. If not, we will include an exogenous Cas3-encoding nucleotide sequence (and optionally one or more nucleotide sequences encoding one or more required Cascade components) in the vector for expression in the target Nissle. We will transform the vector into the TOP10 production strain, induce the P4 anti-repressor and harvest phage comprising the CRISPR/Cas component(s).

Since the induced (helper) phage DNA does not contain a packaging signal we will be able to isolate particles with only the vector DNA packaged. Thus, we will obtain a composition comprising such phage which can be used to infect target Nissle bacteria and introduce the CRISPR/Cas component(s) therein for killing the target bacteria.

### Example 2: MGEs, Genomics Islands Etc

Overview of possible different MGE packaging strategies follow.

Applicable to different types of phages:
Identify packaging signal and structural genes in the helper phage (delivery vehicle)
Delete packaging signal in helper phage and place on plasmid comprising MGE
Place both helper and plasmid in production strain
Induce structural gene transcription of helper to get production of helper-phage-packaged MGEs

For using parasitic mobile elements (P4 phage or SaPI etc) activation of helper phage structural genes is done by induction of a helper phage activator obtained from the parasitic element Delta in P4 or one, more or al of ptiA/B/M in SaPI.

If one wants smaller size particle one can choose to package in a parasite-size capsid (typically 10-20 kb) by including in the MGE or vector P4 Sid and psu or cpmA/B from a SaPI.

One can use defective helper phages where at least the packaging signal has been removed and structural genes are either on a plasmid or integrated as a cryptic prophage in the production host. If for some reason one cannot use this approach and need to use functional helper phages, one will include in the MGE or vector the genes on the parasite that hijack the phage packaging machinery to preferentially package parasite DNA (in our case CGV) over phage DNA.

List of the Minimal Genes One could Include on a Plasmid Vector from P4.

P4 sequence: see world wide web.ncbi.nlm.nih.gov/nuccore/x51522

Cos packaging site (SEQ ID NO: 3):
GCATGCGTTTTCCTGCCTCATTTTCTGCAAACCGCGCCATTCCCGGCGC
GGTCTGAGCGTGTCAGTGCAACTGCATTAAAACCGCCCCGCAAAGCGGG
CGGGCGAGGCGGGGAAAGCACCGCGCGCAAACCGACAAGTTAGTTAATT
ATTTGTGTAGTCAAAGTGCCTTCAGTACATACCTCGTTAATACATTGGA
GCATAATGAAGAAAATCTATGGCCTATGGTC

The homology between P2 and P4 pasted below; this may be used as a packaging signal in the MGE or vector:

(SEQ ID NO: 4)
TGCATTAAAACCGCCCCGCAAAGCGGGCGGGCGAGGCGGGGAAAGCACC
GCGCGC

For small capsid size (packages 11.4 kb instead of 33.5 kb) Sid and/or Psu can be included in the MGE or vector:—

Sid (SEQ ID NO: 5):
ATGTCTGACCACACTATCCCTGAATATCTGCAACCCGCACTGGCACAAC
TGGAAAAGGCCAGAGCCGCCCATCTTGAGAACGCCCGCCTGATGGATGA
GACCGTCACGGCCATTGAACGGGCAGAGCAGGAAAAAAATGCGCTGGCG
CAGGCCGACGGAAACGACGCTGACGACTGGCGCACGGCCTTTCGTGCAG
CCGGTGGTGTCCTGAGCGACGAGCTGAAACAGCGCCACATTGAGCGCGT
GGCACGCCGGGAGCTGGTACAGGAATATGACAATCTGGCCGTGGTGCTG
AATTTCGAACGTGAACGCCTGAAAGGGGCGTGTGACAGCACGGCCACCG
CCTACCGGAAGGCACATCATCACCTTCTGAGTCTGTATGCAGAGCATGA
GCTGGAACACGCCCTGAATGAAACCTGTGAGGCGCTTGTCCGGGCAATG
CATCTGAGCATTCTGGTACAGGAAAATCCGCTCGCCAACACCACCGGCC
ATCAGGGCTACGTCGCACCGGAAAAGGCTGTCATGCAGCAGGTGAAATC
ATCGCTGGAACAGAAAATTAAACAGATGCAAATCAGCCTCACCGGCGAG
CCGGTTCTCCGGCTGACCGGACTGTCAGCGGCAACACTCCCGCACATGG
ATTATGAGGTGGCAGGCACACCGGCACAGCGCAAGGTGTGGCAGGACAA
AATAGACCAGCAGGGAGCAGAGCTTAAGGCCAGAGGGCTGCTGTCATGA
Psu (SEQ ID NO: 6):
ATGGAAAGCACAGCCTTACAGCAGGCCTTTGACACCTGTCAGAATAACA
AAGCAGCATGGCTGCAACGCAAAAATGAGCTGGCAGCGGCCGAACAGGA
ATATCTGCGGCTTCTGTCAGGAGAAGGCAGAAACGTCAGTCGCCTGGAC
GAATTACGCAATATTATCGAAGTCAGAAAATGGCAGGTGAATCAGGCCG
CCGGTCGTTATATTCGTTCGCATGAAGCCGTTCAGCACATCAGCATCCG
CGACCGGCTGAATGATTTTATGCAGCAGCACGGCACAGCACTGGCGGCC
GCACTGGCACCGGAGCTGATGGGCTACAGTGAGCTGACGGCCATTGCCC
GAAACTGTGCCATACAGCGTGCCACAGATGCCCTGCGTGAAGCCCTTCT
GTCCTGGCTTGCGAAGGGTGAAAAAATTAATTATTCCGCACAGGATAGC
GACATTTTAACGACCATCGGATTCAGGCCTGACGTGGCTTCGGTGGATG
ACAGCCGTGAAAAATTCACCCCTGCGCAGAACATGATTTTTTCGCGTAA
AAGTGCGCAACTGGCATCACGTCAGTCAGTGTAA

To activate helper phage P2, Delta can be included in a host cell genome (provided separately in a host cell, not on the MGE or vector to be packaged)

Delta (SEQ ID NO: 7):
ATGATTTACTGTCCGTCGTGTGGACATGTTGCTCACACCCGTCGCGCAC
ATTTCATGGACGATGGCACCAAGATAATGATTGCACAGTGCCGGAATAT
TTATTGCTCTGCGACATTTGAAGCGAGTGAAAGCTTTTTCTCTGACAGT
AAAGATTCAGGAATGGAATACATTTCAGGCAAACAGAGATACCGCGATT
CACTGACGTCAGCCTCCTGCGGTATGAAACGCCCGAAAAGAATGCTTGT
TACCGGATATTGTTGTCGGAGATGTAAAGGCCTTGCACTGTCAAGAACA
TCGCGGCGTCTGTCTCAGGAAGTCACCGAGCGTTTTTATGTGTGCACGG
ATCCGGGCTGTGGTCTGGTGTTTAAAACGCTTCAGACCATCAACCGCTT
CATTGTCCGCCCGGTCACGCCGGACGAACTGGCAGAACGCCTGCATGAA
AAACAGGAACTGCCGCCAGTACGGTTAAAAACACAATCATATTCGCTGC
GTCTGGAATGA

Minimum Genes to Include in the Host Chromosome/Episome from P2.

P2 sequence (acc. number: NC_001895)

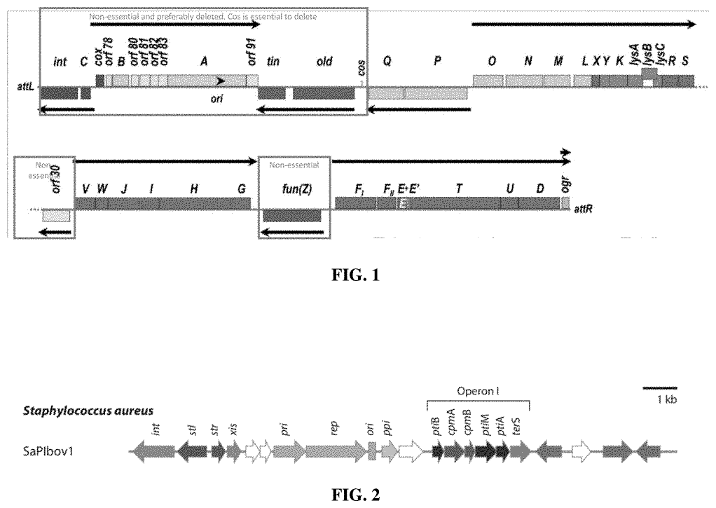

FIG. 1 shows a genetic map of P2 genome with non-essential genes boxed in red—one, more or all of these can be excluded. Cos is deleted and preferably the whole region from int through cos. This region may, for example, be swapped with a resistance marker while the orf30 and fun(Z) genes are left intact.

“Q” through “S”
(SEQ ID NO: 8)
TCAGTCGTTGTCAGTGTCCAGTGAGTAGTTTTTAAAGCGGATGACCTCCTGACCGAGCCAGC
CGTTTATCTCGCGGATCCTGTCCTGTAACGGGATAAGCTCATTGCGGACAAAGACCTTTGCC
ACTTTCTCAATATCACCCAGCGACCCGACGTTCTCCGGCTTGCCACCCATCAACTGAAAGGG
GATGCGGTGCGCGTCCAGCAGGTCAGCGGCGCTGGCTTTTTTGATATTAAAAAAATCGTCCT
TCGTCGCCACTTCACTGAGGGGGATAATTTTAATGCCGTCGGCTTTCCCCTGTGGGGCATAG
AGAAACAGGTTTTTAAAGTTGTTGCGGCCTTTCGACTTGACCATGTTTTCGCGAAGCATTTCG
ATATCGTTGCGATCCTGCACGGCATCGGTGACATACATGATGTATCCGGCATGTGCGCCATT
TTCGTAATACTTGCGGCGGAACAACGTGGCCGACTCATTCAGCCAGGCAGAGTTAAGGGCG
CTGAGATATTCCGGCAGGCCGTACAGCTCCTGATTAATATCCGGCTCCAGCAGGTGAAACAC
GGAGCCGGGCGCGAAGGCTGTCGGCTCGTTGAAGGACGGCACCCACCAGTAAACATCCTCT
TCCACGCCACGGCGGGTATATTTTGCCGGTGAGGTTTCCAGTCTGATGACCTTACCGGTGGT
GCTGTAACGCTTTTCCAGAAACGCATTACCGAACACCAGAAAATCCAGCACAAAGCGGCTG
AAATCCTGCTGGGAAAGCCATGGATGCGGGATAAATGTCGAGGCCAGAATATTGCGTTTGA
CGTAAATCGGCGAGCTGTGATGCACGGCAGCCCGCAGGCTTTTTGCCAGACCGGTAAAGCTG
ACCGGTGGCTCATACCATCTGCCGTTACTGATGCACTCGACGTAATCCAGAATGTCACGGCG
GTCGAGTACCGGCACCGGCTCACCAAAGGTGAATGCCTCCATTTTCGGGCCGCTGGCGGTCA
TTGTTTTTGCCGCAGGTTGCGGTGTTTTCCCTTTTTTCTTGCTCATCAGTAAAACTCCAGAATG
GTGGATGTCAGCGGGGTGCTGATACCGGCGGTGAGTGGCTCATTTAACAGGGCGTGCATGGT
CGCCCAGGCGAGGTCGGCGTGGCTGGCTTCCTCGCTGCGGCTGGCCTCATAGGTGGCGCTGC
GTCCGCTGCTGGTCATGGTCTTGCGGATAGCCATAAACGAGCTGGTGATGTCGGTGGCGCTG
ACGTCGTATTCCAGACAGCCACGGCGGATAACGTCTTTTGCCTTGAGCACCATTGCGGTTTTC
ATTTCCGGCGTGTAGCGGATATCACGCGCGGCGGGATAGAACGAGCGCACGAGCTGGAACA
CGCCGACACCGAGGCCGGTGGCATCAATACCGATGTATTCGACGTTGTATTTTTCGGTGAGT
TTGCGGATGGATTCCGCCTGGGTGGCAAAGTCCATGCCTTTCCACTGGTGACGCTCAAGTAT
TCTGAATTTGCCACCGGCCACCACCGGCGGTGCCAGTACCACGCATCCGGCGCTGTCGCCAC
GGTGTGACGGGTCGTAACCAATCCATACCGGGCGGGAGCCGAACGGATTGGCGGCAAACGG
CGCATAGTCTTCCCATTCTTCCAGCGTGTCGACCATGCAGCGTTGCAGCTCCTCGAACGGGA
ACACCGATGCCTTGTCGTCAACAAATTCACACATGAACAGGTTTTTAAAATCGTCGGCGCTG
TTTTCGCGTTTGAGCTGCTCAATGTCGAACAGCGTGCAGCCGCCTTTCAGGGCGTCCTCAATG
GTGACAATCTGTCGCCACTGGCCGTCCGCACAGAGAAGCCCACCGGCAAGGGCGTTATGAC
TGACGTCGATTTCCACGCGTTCGGCGGCGCTGGCGCGTCCCCGGTTAAACAGTTCACCCGAC
CAGAACGGGTAGGCGTCGTGCGCCAGCGTGGACGGGGTGGAGAAATAGGTCGAGCGCAGGT
GACTCTGTGAGGCCATACCTGATGCCACCTTACGCAGTACCTGAAAATTCGGGATCCAGAAA
ATCTCATCGACGTACAGGTCGCCGTTATGACTCTGCGCGGTGTTGGAGTTGGTGCCGAGAAA
AATCAGTTTTGCGCCGTTATTGCCCAGGACAATCGGGTCACCGGTCAGGTCAACGTCAACCA
GACGGGCAAAGGCGATGATGTATTCGCGGAACACATACGCCTGTGTTTTACTGGCCGACAG
AAAAATCTGGTTATGACCGGTTTTCAGGGCACGCAGCAGCGCCTCGCGGGAAAAATAAAAC
GTCGCGCCAATCTGGCGGGATTTCAGGATATCGCGGATGCGGTGCTCAAGCCCGGCACGATA
CCAGTGCAGCTGATAGTCGAAAGACTGCTCAAAGAAAATCTGCTCCAGCTTTTCGATGGCCT
CGTCACTGAAAAAATTCTTTTTCGGTTTGCGCCGTCCGCCTTTGTTACGGTTAGCGACGTTCG
GATTAAGGTCTGCCTCGTTGCCGGTCTGGCTGTAGCGGTTGACCCGTGCCAGTCGTTCAATCT
GGCGTCCCAGCAGGTCAATTTCCTTGAAGTCACCGCCGGTTTTCTGTGGTTTGATGATGAGCT
GGGTCAGCCGCGCTTCCAGACTCATTTCGACACGGCTGATGGGGGCAACGCTGTCCCAGCCG
TCGCGCTGTTTCCAGCTCTGCACTGTCGGGCGTTTCATCTGCAACATGGCGGCAATCTGCGGC
ACGGAAAACCCCTGCCAGTACAGCAGCGCCGCCTGACGACGCGGGTCGTGTAAAAGAGTGG
TGTCTGTGGTGATGGTCATGAATACCTCGCCGTGATGAATACACGGCAAGGCTACTGAGTCG
CGCCCCGCGATTCGCTAAGGTGCTGTTGTGTCAGTGATAAGCCATCCGGGACTGATGGCGGA
GGATGCGCATCGTCGGGAAACTGATGCCGACATGTGACTCCTCTAATCACTATTCAGGACTC
CTGACAATGGCAAAAAAAGTCTCAAAATTCTTTCGTATCGGCGTTGAGGGTGACACCTGTGA
CGGGCGTGTCATCAGTGCGCAGGATATTCAGGAAATGGCCGAAACCTTTGACCCGCGTGTCT
ATGGTTGCCGCATTAACCTGGAACATCTGCGCGGCATCCTGCCTGACGGTATTTTTAAGCGTT
ATGGCGATGTGGCCGAACTGAAGGCCGAAAAGATTGACGATGATTCGGCGCTGAAAGGCAA
ATGGGCGCTGTTTGCGAAAATCACCCCGACCGATGACCTTATCGCGATGAACAAGGCCGCGC
AGAAGGTCTACACCTCAATGGAAATTCAGCCGAACTTTGCCAACACCGGCAAATGTTATCTG
GTGGGTCTGGCCGTCACCGATGACCCGGCAAGCCTCGGCACGGAATACCTGGAATTCTGCCG
CACGGCAAAACACAACCCCCTGAACCGCTTCAAATTAAGCCCTGAAAACCTGATTTCAGTGG
CAACGCCTGTTGAGCTGGAATTTGAAGACCTGCCTGAAACCGTGTTCACCGCCCTGACCGAA
AAGGTGAAGTCCATTTTTGGCCGCAAACAGGCCAGCGATGATGCCCGTCTGAATGACGTGCA
TGAAGCGGTGACCGCTGTTGCTGAACATGTGCAGGAAAAACTGAGCGCCACTGAGCAGCGC
CTCGCTGAGATGGAAACCGCCTTTTCTGCACTTAAGCAGGAGGTGACTGACAGGGCGGATG
AAACCAGCCAGGCATTCACCCGCCTGAAAAACAGTCTCGACCACACCGAAAGTCTGACCCA
GCAGCGCCGCAGCAAAGCCACCGGCGGTGGCGGTGACGCCCTGATGACGAACTGCTGACCG
GCGTCAGTCAGTCCGGGAAAACCTTCACGATTAACCCTTAATTTCAGGAAAAACTATGCGCC
AGGAAACCCGCTTTAAATTTAATGCCTACCTGTCCCGTGTTGCCGAACTGAACGGCATCGAC
GCCGGTGATGTGTCGAAAAAATTCACCGTTGAACCGTCGGTCACCCAGACCCTGATGAACAC
CATGCAGGAGTCCTCTGACTTTCTGACCCGCATCAATATTGTGCCGGTCAGCGAAATGAAAG
GGGAAAAAATTGGTATCGGTGTCACCGGCTCCATCGCCAGCACTACCGACACTGCCGGTGGT
ACCGAGCGTCAGCCGAAGGACTTCTCGAAGCTGGCGTCAAACAAGTACGAATGCGACCAGA
TTAACTTCGATTTTTATATCCGCTACAAAACGCTGGACCTGTGGGCGCGTTATCAGGATTTCC
AGCTCCGTATCCGTAACGCCATTATCAAACGCCAGTCCCTTGATTTCATCATGGCCGGTTTTA
ACGGCGTGAAGCGTGCCGAAACCTCTGACCGCAGCAGCAATCCGATGTTGCAGGATGTGGC
GGTCGGCTGGCTGCAGAAATACCGCAATGAAGCACCGGCGCGCGTGATGAGCAAGGTCACT
GACGAGGAAGGCCGCACCACCTCTGAGGTTATCCGCGTGGGTAAGGGCGGTGATTATGCCA
GCCTTGATGCACTGGTGATGGATGCGACCAACAACCTGATTGAACCGTGGTATCAGGAAGA
CCCTGACCTTGTGGTGATTGTGGGGCGTCAGCTACTGGCGGACAAGTATTTCCCCATCGTCA
ACAAGGAGCAGGACAACAGCGAAATGCTGGCCGCTGACGTCATCATCAGCCAGAAACGCAT
CGGTAACCTACCAGCGGTACGCGTCCCGTACTTCCCGGCGGATGCGATGCTCATCACGAAGC
TGGAAAACCTGTCCATCTACTACATGGATGACAGCCATCGCCGCGTGATTGAGGAAAACCCG
AAACTCGACCGCGTGGAGAACTACGAGTCAATGAACATTGATTACGTGGTGGAAGACTACG
CCGCCGGTTGTCTGGTGGAAAAAATCAAGGTCGGTGACTTCTCCACACCGGCTAAGGCGACC
GCAGAGCCGGGAGCGTAACCGATGACGAGTCCCGCACAGCGCCACATGATGCGGGTCTCGG
CAGCGATGACCGCGCAGCGGGAAGCCGCCCCGCTGCGACATGCAACTGTCTATGAGCAGAT
GCTGGTTAAGCTCGCCGCAGACCAGCGCACACTGAAAGCGATTTACTCAAAAGAGCTGAAG
GCCGCAAAAAAACGCGAACTGCTGCCGTTCTGGTTGCCGTGGGTGAACGGCGTGCTGGAGC
TGGGCAAAGGTGCACAGGATGACATTCTGATGACGGTCATGCTGTGGCGTCTGGATACCGGC
GATATTGCCGGTGCGCTGGAGATTGCCCGTTATGCCCTGAAGTACGGTCTGACCATGCCGGG
TAAACACCGCCGTACCCCGCCGTACATGTTCACCGAGGAGGTAGCGCTTGCGGCCATGCGCG
CTCACGCTGCCGGTGAGTCTGTGGATACCCGCCTGCTGACGGAGACCCTTGAACTGACCGCC
ACGGCTGACATGCCTGATGAAGTGCGCGCAAAGCTGCACAAAATCACCGGTCTGTTTCTGCG
TGACGGTGGTGATGCCGCCGGTGCGCTGGCGCACCTGCAACGTGCGACACAGCTCGACTGTC
AGGCAGGCGTCAAAAAAGAGATTGAACGACTGGAGCGGGAGCTGAAACCGAAGCCGGAGC
CGCAGCCCAAAGCGGCCACCCGCGCCCCGCGTAAGACCCGGAGCGTGACACCGGCAAAACG
TGGACGCCCGAAAAAGAAAGCCAGTTAACAACCGAATGCGCCCCGCGCCAGGGCGGCACGC
CGGTCAGTGACGGTGAATCACCTGACACTGCACCGGCGTCCACCGCCCGACTTTTCAGAGGT
AGTCATGATGACGCTGATTATTCCGCGAAAGGAGGCTCCCGTGTCCGGTGAGGGTACGGTGG
TCATCCCGCAACCGGCAGGCGACGAGCCGGTGATTAAAAACACGTTCTTTTTTCCCGATATC
GACCCGAAGCGCGTCCGGGAACGTATGCGCCTTGAGCAGACCGTCGCCCCCGCCCGTCTGCG
TGAGGCCATCAAGTCAGGCATGGCTGAAACGAATGCGGAGCTGTACGAGTACCGCGAACAG
AAAATTGCCGCCGGTTTTACGCGTCTGGCTGACGTCCCGGCGGACGATATCGACGGTGAAAG
CATCAAGGTTTTTTACTACGAGCGCGCCGTGTGTGCGATGGCGACCGCGTCGCTTTATGAGC
GTTATCGCGGTGTGGATGCCAGTGCGAAAGGCGACAAGAAGGCTGACAGCATTGACAGCAC
CATTGATGAGCTGTGGCGGGATATGCGCTGGGCGGTGGCGCGCATCCAGGGCAAGCCGCGC
TGCATCGTGAGTCAAATCTGATGAAGACCTTTGCGCTACAGGGCGACACGCTCGACGCCATT
TGTGTCCGCTATTACGGGCGCACTGAGGGCGTGGTTGAGACCGTGCTCGCCGCAAATCCGGG
ACTGGCTGAACTGGGGGCGGTGCTGCCACACGGCACCGCCGTCGAACTGCCCGACGTTCAG
ACCGCGCCCGTGGCTGAAACTGTCAATCTGTGGGAGTAACGCATGACAGCAGAAGAAAAAA
GCGTCCTGTCGCTTTTCATGATTGGGGTGCTGATTGTTGTCGGCAAGGTGCTTGCCGGTGGTG
AACCTATCACCCCGCGTCTGTTTATCGGGCGCATGTTGCTCGGTGGTTTTGTCTCGATGGTTG
CCGGTGTTGTTCTGGTGCAGTTTCCTGACCTGTCACTGCCAGCGGTGTGCGGCATCGGCTCCA
TGCTGGGTATCGCCGGTTATCAGGTGATTGAGATTGCCATTCAGCGCCGCTTTAAGGGCAGG
GGGAAACAGTAATGCCGGTAATTAACACGCATCAGAATATCGCCGCCTTTCTCGACATGCTG
GCCGTGTCCGAAGGGACGGCGAATCATCCACTGACGAAAAACCGGGGCTATGACGTGATAG
TCACCGGACTGGACGGGAAGCCGGAAATTTTCACCGACTACAGTGACCACCCGTTCGCACAT
GGCCGACCGGCGAAGGTGTTTAACCGTCGCGGTGAAAAATCCACGGCCTCCGGTCGCTATCA
GCAGCTTTACCTGTTCTGGCCGCATTACCGCAAACAGCTTGCCCTGCCGGATTTCAGTCCGTT
GTCACAGGACAGACTCGCCATTCAGTTGATCCGCGAACGCGGAGCACTGGATGACATCCGG
GCGGGACGCATTGAGCGCGCCATTTCACGCTGTCGCAATATCTGGGCGTCCCTGCCGGGTGC
CGGTTACGGTCAGCGTGAGCATTCACTGGAAAAACTGGTCACCGTCTGGCGTACCGCTGGCG
GCGTACCGGCTTAAACGGAGTAAATACCATGAAGAAATTATCCCTTTCACTGATGCTGAACG
TGTCGCTGGCGCTGATGCTGGCACTGTCCCTGATTTACCCGCAGAGCGTGGCCGTCAATTTTG
TCGCTGCCTGGGCGATTCTGGCGACGGTTATCTGTGTGGTTGCCGGTGGTGTGGGCGTGTAT
GCCACTGAGTATGTGCTGGAACGCTACGGGCGGGAGCTGCCGCCGGAATCGCTGGCCGTGA
AGATTGTCACGTCGCTGTTTTTGCAGCCGGTGCCGTGGCGCAGACGGGCGGCGGCTCTGGTA
GTGGTGGTGGCGACGTTTATCTCGCTGGTCGCTGCCGGGTGGATTTTTACCGCGCTGATTTAT
CTTGTGGTGTCGCTGTTTTTCCGGCTGATACGTAAAGCCTGTCGTCAGCGTCTTGAGGGGCGG
GAACCATGTCAAGGCTGATGATTGTGCTGGTCGTGTTGTTATCGCTGGCGGTGGCCGGTCTG
TTTCTGGTGAAACACAAAAATGCCAGCCTGCGCGCCTCGCTGGACAGGGCGAACAACGTCG
CCAGCGGTCAGCAGACGACCATCACCATGCTGAAAAATCAGCTTCATGTTGCGCTCACCAGG
GCAGATAAAAACGAGCTGGCGCAGGTGGCACTGCGTCAGGAACTGGAGAACGCCGCGAAA
CGTGAAGCACAGCGCGAGAAAACCATCACGAGGTTACTTAATGAGAACGAAGATTTTCGCC
GCTGGTACGGTGCTGACCTGCCTGATGCTGTGCGCCGGTTGCACCAGCGCCCCGCCTGCACC
GACGCCAGTGATTGTCCCCAACGCATGCCCGAAAGTGAGCCTTTGCCCGATGCCGGGCAGTG
ACCCGCAGACGAACGGCGATTTAAGTGCCGATATCCGGCAGCTTGAGAACGCGCTGGCACG
CTGTGCCAGCCAGGTAAAAATGATTAAACACTGTCAGGACGAAAACGATGCTCAAACCCGA
CAGCCTGCGCAGGGCGCTGACTGATGCCGTCACGGTGCTGAAAACTAACCCCGATATGCTGC
GGATATTCGTGGATAACGGGAGTATTGCCTCCACACTGGCGGCGTCGCTGTCATTCGAAAAG
CGTTACACGCTCAATGTGATTGTGACCGACTTTACCGGTGATTTTGACCTGCTCATTGTGCCG
GTGCTGGCGTGGCTGCGGGAAAATCAGCCCGACATCATGACCACCGACGAAGGCCAGAAAA
AGGGCTTCACGTTTTATGCAGACATCAACAATGACAGCAGCTTTGATATCAGTATCAGCCTG
ATGCTGACCGAGCGCACGCTGGTCAGTGAGGTGGACGGCGCACTGCATGTGAAGAATATCT
CGGAACCCCCGCCGCCGGAGCCGGTCACCCGCCCGATGGAGCTGTATATCAATGGCGAACT
GGTGAGTAAGTGGGATGAATGAGTTTAAGCGTTTTGAAGACCGGCTGACCGGACTGATTGA
ATCGCTGTCACCGTCAGGGCGTCGGCGACTGAGTGCCGAACTGGCGAAACGTCTGCGGCAG
AGTCAGCAGCGTCGGGTGATGGCACAGAAAGCCCCGGACGGCACACCCTACGCGCCACGCC
AGCAGCAGAGCGTCAGAAAAAAGACCGGTCGCGTTAAGCGAAAAATGTTTGCGAAACTTAT
TACCAGTCGTTTTTTGCATATCCGTGCCAGCCCGGAGCAGGCATCAATGGAATTTTACGGCG
GGAAGTCGCCGAAAATCGCCAGTGTGCATCAGTTTGGTCTGTCGGAAGAAAACCGGAAAGA
CGGTAAGAAAATTGATTATCCGGCGCGTCCCCTGCTCGGCTTTACCGGTGAGGATGTGCAGA
TGATTGAAGAGATTATCCTGGCTCACCTTGAGCGTTAG
“V” through “G” (SEQ ID NO: 9):
ATGAACACTCTCGCAAATATTCAGGAACTCGCGCGCGCACTGCGCAACATGATTCGCACTGG
CATTATCGTCGAAACCGACCTTAACGCCGGTCGCTGCCGCGTGCAGACCGGCGGCATGTGCA
CCGACTGGCTTCAGTGGCTGACCCATCGCGCAGGACGTTCGCGCACATGGTGGGCACCTTCC
GTGGGGGAACAGGTGCTGATTCTGGCCGTGGGTGGTGAACTCGACACGGCGTTCGTTCTGCC
GGGGATTTATTCCGGCGATAACCCCTCGCCGTCTGTGTCGGCGGATGCCCTGCATATCCGTTT
CCCTGACGGGGCGGTGATTGAATATGAACCCGAAACCAGTGCACTCACGGTAAGCGGAATT
AAAACGGCCAGCGTGACGGCTTCCGGTTCTGTTACTGCCACGGTGCCGGTGGTCATGGTGAA
AGCATCAACCCGCGTCACCCTGGACACCCCGGAGGTGGTCTGCACCAACAGGCTGATTACCG
GCACGCTGGAAGTGCAGAAAGGCGGGACGATGCGCGGCAACATTGAACACACCGGCGGTG
AACTCTCATCAAACGGTAAGGTACTGCATACCCATAAACACCCCGGCGACAGCGGCGGCAC
AACCGGGAGTCCTTTATGACAGCGCGTTATCTCGGAATGAATCGCAGTGATGGCCTGACTGT
CACTGACCTTGAGCATATCAGCCAGAGTATCGGCGATATCCTGCGCACACCGGTCGGCTCAC
GGGTGATGCGTCGTGATTACGGCTCGTTGCTGGCGTCAATGATTGACCAGCCGCAGACCCCG
GCGCTTGAGTTGCAGATTAAAGTCGCCTGTTACATGGCAGTGCTGAAATGGGAACCCCGCGT
CACCCTGTCATCCGTCACCACGGCGCGCAGTTTTGACGGGCGAATGACGGTCACGTTAACCG
GCCAGCACAACGACACCGGCCAGCCACTTTCATTAACCATCCCTGTGAGTTGAAACCATGCC
GATTATCGACCTGAACCAGCTACCCGCACCGGATGTGGTCGAGGAGCTGGACTTTGAAAGC
ATTCTCGCTGAACGCAAGGCGACACTGATTTCCCTTTACCCGGAAGATCAGCAGGAGGCGGT
CGCCCGTACCCTGACACTGGAATCTGAGCCTCTCGTCAAACTGCTGGAAGAAAATGCTTATC
GTGAGCTTATCTGGCGTCAGCGTGTGAATGAGGCCGCACGGGCGGTGATGCTGGCCTGTGCC
GCCGGTAATGACCTTGATGTGATTGGTGCCAATTACAACACCACGCGCCTGACTATCACCCC
GGCAGATGATTCGACCATCCCGCCGACACCGGCAGTGATGGAATCTGACACCGATTATCGTC
TGCGTATTCAGCAGGCTTTTGAGGGCTTAAGCGTCGCCGGGTCAGTGGGAGCCTATCAGTAT
CATGGTCGCAGTGCTGACGGGCGTGTCGCGGATATTTCTGTCACCAGTCCGTCTCCGGCCTG
TGTCACCATCTCTGTGCTGTCACGTGAAAATAACGGCGTCGCATCCGAAGACCTGCTGGCTG
TGGTGCGTAACGCCCTTAATGGCGAGGACGTCAGGCCGGTGGCCGACCGCGTGACCGTGCA
GTCTGCCGCCATCGTTGAATACCAGATAAACGCCACGCTTTACCTTTACCCTGGTCCCGAAA
GCGAACCCATCCGCGCTGCCGCTGTGAAAAAGCTGGAAGCGTATATCACGGCACAGCACCG
GCTGGGGCGCGACATCCGTCTGTCTGCCATTTATGCCGCTTTGCATGTGGAAGGTGTGCAGC
GTGTCGAACTGGCTGCACCACTGGCCGACATCGTGCTCAACAGTACGCAGGCGTCTTTCTGT
ACCGAATACCGCGTCGTGACCGGAGGCTCGGATGAGTGATTCGCGACTGCTGCCGACCGGCT
CATCACCGCTTGAGGTCGCCGCCGCAAAAGCCTGTGCGGAAATTGAAAAAACGCCGGTCAG
TATTCGTGAACTGTGGAACCCGGACACCTGTCCGGCAAATCTGCTGCCGTGGCTGGCGTGGG
CGTTTTCGGTCGACAGGTGGGATGAAAAGTGGCCGGAAGCGACAAAACGCGCCGTTATCCG
CGATGCCTATTTCATCCACTGTCATAAGGGCACGATAGGTGCAATCCGGCGTGTGGTGGAGC
CGCTCGGCTATCTCATCAACGTGACGGAGTGGTGGGAAAACAGTGACCCGCCCGGCACCTTC
CGGCTTGATATTGGTGTACTGGAAAGCGGTATCACAGAGGCAATGTATCAGGAAATGGAAC
GGCTGATTGCTGATGCCAAACCTGCAAGCCGTCACCTTATTGGCCTGAACATTACCCGGGAC
ATTCCCGGCTATCTGTTCGCCGGTGGTGTGGCTTACGACGGCGATGTAATTACGGTTTACCCC
GGATAAGTGAGGAATAATGAGCATAAAATTCAGAACCGTTATCACCACTGCCGGTGCAGCA
AAGCTGGCAGCGGCAACCGCGCCGGGAAGGCGGAAGGTCGGCATTACCACGATGGCCGTCG
GGGATGGCGGTGGTAAATTGCCTGTCCCGGATGCCGGACAGACCGGGCTTATCCATGAAGTC
TGGCGACATGCGCTGAACAAAATCAGCCAGGACAAACGAAACAGTAATTATATTATCGCCG
AGCTGGTTATTCCGCCGGAGGTGGGCGGTTTCTGGATGCGTGAGCTTGGCCTGTACGATGAT
GCGGGAACGTTAATTGCCGTGGCGAACATGGCCGAAAGCTATAAGCCAGCCCTTGCCGAAG
GCTCAGGACGTTGGCAGACCTGTCGCATGGTCATCATCGTCAGCAGTGTGGCCTCAGTGGAG
CTGACCATTGACACCACAACGGTGATGGCGACGCAGGATTACGTTGATGACAAAATTGCAG
AGCACGAACAGTCACGACGTCACCCGGACGCCTCGCTGACAGCAAAAGGTTTTACTCAGTTA
AGCAGTGCGACCAACAGCACGTCTGAAACACTGGCCGCAACGCCGAAAGCGGTAAAGGCCG
CGTATGACCTGGCTAACGGGAAATATACCGCACAGGACGCCACCACAGCGCGAAAAGGCCT
TGTCCAGCTTAGTAGCGCCACCAACAGCACGTCTGAAACGCTCGCCGCAACACCAAAAGCC
GTTAAGACGGTAATGGATGAAACGAACAAAAAAGCGCCATTAAACAGCCCTGCACTGACCG
GAACGCCAACGACGCCAACTGCGCGACAGGGAACGAATAATACTCAGATCGCAAACACGGC
TTTCGTTATGGCCGCGATTGCCGCCCTTGTAGACTCGTCGCCTGACGCACTGAATACGCTGA
ACGAGCTGGCGGCGGCGCTGGGCAATGACCCGAATTTTGCTACCACCATGACTAATGCGCTT
GCGGGTAAGCAACCGAAAGATGCTACCCTGACGGCGCTGGCGGGGCTTGCTACTGCGGCAG
ACAGGTTTCCGTATTTTACGGGGAATGATGTTGCCAGCCTGGCGACCCTGACAAAAGTCGGG
CGGGATATTCTGGCTAAATCGACCGTTGCCGCCGTTATCGAATATCTCGGTTTACAGGAAAC
GGTAAACCGAGCCGGGAACGCCGTGCAAAAAAATGGCGATACCTTGTCCGGTGGACTTACT
TTTGAAAACGACTCAATCCTTGCCTGGATTCGAAATACTGACTGGGCGAAGATTGGATTTAA
AAATGATGCCGATGGTGACACTGATTCATACATGTGGTTTGAAACGGGGGATAACGGCAAT
GAATATTTCAAATGGAGAAGCCGCCAGAGTACCACAACAAAAGACCTGATGACGTTGAAAT
GGGATGCACTAAATATTCTTGTTAATGCCGTCATTAATGGCTGTTTTGGAGTTGGTACGACG
AATGCACTAGGTGGTAGCTCTATTGTTCTTGGTGATAATGATACCGGATTTAAACAGAATGG
AGACGGTATTCTTGATGTTTATGCTAACAGTCAGCGTGTATTCCGTTTTCAGAATGGAGTGGC
TATTGCTTTTAAAAATATTCAGGCAGGTGATAGTAAAAAGTTCTCGCTATCCAGCTCTAATA
CATCCACGAAGAATATTACCTTTAATTTATGGGGTGCTTCCACCCGTCCAGTGGTTGCAGAG
TTAGGCGATGAGGCCGGATGGCATTTCTATAGCCAGCGAAATACAGATAACTCGGTAATATT
TGCTGTTAACGGTCAGATGCAACCCAGCAACTGGGGAAATTTTGATTCCCGCTATGTGAAAG
ATGTTCGCCTGGGTACGCGAGTTGTTCAATTGATGGCGCGAGGTGGTCGTTATGAAAAAGCC
GGACACACGATTACCGGATTAAGAATCATTGGTGAAGTAGATGGCGATGATGAAGCCATCT
TCAGGCCGATACAAAAATACATCAATGGCACATGGTATAACGTTGCGCAGGTGTAAGTTATG
CAGCATTTAAAGAACATTAAGTCAGGTAATCCAAAAACAAAAGAGCAATATCAGCTAACAA
AGAATTTTGATGTTATCTGGTTATGGTCCGAAGACGGAAAAAACTGGTATGAGGAAGTGAA
GAACTTTCAGCCAGACACAATAAAGATTGTTTACGATGAAAATAATATTATTGTCGCTATCA
CCAGAGATGCTTCAACGCTTAATCCTGAAGGTTTTAGCGTTGTTGAGGTTCCTGATATTACCT
CCAACCGACGTGCTGACGACTCAGGTAAATGGATGTTTAAGGATGGTGCTGTGGTTAAACGG
ATTTATACGGCAGATGAACAGCAACAACAGGCAGAATCACAAAAGGCCGCGTTACTTTCCG
AAGCGGAAAACGTTATTCAGCCACTGGAACGCGCTGTCAGGCTGAATATGGCGACGGATGA
GGAACGTGCACGACTGGAGTCATGGGAACGTTACAGCGTTCTGGTCAGCCGTGTGGATCCTG
CAAATCCTGAATGGCCGGAAATGCCGCAATAA
“FI” through “ogr” (SEQ ID NO: 10)
ATGAGTGACTATCATCACGGCGTGCAGGTGCTGGAGATTAACGAGGGCACCCGCGTCATTTC
CACCGTATCCACGGCCATTGTCGGCATGGTCTGCACGGCCAGCGATGCAGATGCGGAAACCT
TCCCCCTCAATAAACCTGTGCTGATTACCAATGTGCAGAGCGCAATTTCAAAGGCCGGTAAA
AAAGGCACGCTGGCGGCATCGTTGCAGGCCATCGCTGACCAGTCAAAACCGGTCACCGTTGT
CATGCGCGTGGAAGACGGCACCGGTGATGACGAGGAAACGAAACTCGCGCAGACCGTTTCC
AATATCATCGGCACCACCGATGAAAACGGTCAGTACACCGGACTAAAAGCCATGCTGGCGG
CGGAGTCGGTAACCGGTGTTAAACCGCGTATTCTCGGCGTGCCGGGACTGGATACCAAAGA
GGTGGCTGTTGCACTGGCATCAGTCTGTCAGAAGCTGCGTGCTTTCGGGTATATCAGCGCAT
GGGGCTGTAAAACCATTTCCGAGGTGAAAGCCTATCGTCAGAATTTCAGCCAGCGTGAGCTG
ATGGTCATCTGGCCGGATTTCCTCGCATGGGATACGGTCACCAGTACCACCGCCACCGCGTA
TGCCACCGCCCGTGCGCTGGGGCTGCGCGCTAAAATCGACCAGGAGCAGGGCTGGCATAAA
ACGCTGTCCAATGTCGGGGTGAACGGTGTTACCGGCATCAGCGCATCTGTATTCTGGGATTT
GCAGGAGTCCGGCACCGATGCTGACCTGCTTAACGAGTCAGGCGTCACTACGCTGATTCGCC
GCGACGGTTTCCGCTTCTGGGGTAACCGTACCTGCTCTGATGACCCGCTGTTCCTCTTTGAAA
ACTACACCCGCACCGCGCAGGTCGTGGCCGACACGATGGCTGAGGCGCACATGTGGGCGGT
GGACAAGCCCATCACTGCAACGCTGATTCGCGACATCGTTGACGGCATCAATGCCAAATTCC
GTGAGCTGAAAACAAACGGCTATATCGTGGATGCGACCTGCTGGTTCAGCGAAGAATCCAA
CGATGCGGAAACCCTCAAGGCCGGAAAACTGTATATCGACTACGACTATACACCGGTGCCTC
CTCTCGAAAACCTGACCCTGCGCCAGCGTATTACCGATAAATACCTGGCAAATCTGGTCACC
TCGGTTAACAGCAATTAAGGAGCCTGACCGATGGCAATGCCGCGCAAACTCAAGTTAATGA
ACGTCTTTCTGAACGGCTACAGCTATCAGGGCGTTGCAAAGTCCGTCACGCTGCCAAAACTG
ACCCGTAAGCTCGAAAACTATCGCGGTGCGGGGATGAACGGCAGCGCACCGGTAGACCTCG
GCCTTGATGACGATGCGCTGTCAATGGAGTGGTCGCTCGGTGGCTTCCCGGATTCGGTTATC
TGGGAGCTTTACGCCGCAACCGGTGTGGATGCCGTGCCGATTCGTTTTGCAGGCTCTTACCA
GCGCGACGATACCGGCGAAACGGTGGCCGTCGAAGTGGTCATGCGTGGACGTCAGAAAGAA
ATCGACACCGGCGAGGGTAAACAGGGAGAAGACACTGAGTCGAAAATCTCCGTGGTCTGCA
CCTATTTCCGGCTGACGATGGACGGTAAGGAGCTGGTCGAAATTGACACCATCAACATGATT
GAGAAGGTGAACGGCGTCGATCGGCTGGAGCAACACCGCCGCAATATCGGCCTGTGATTTT
CATCCGGTCAGCCTGGCTGGCCGGTTAACCCTGATTCAGAAGTGAGAAAACCATGAACAAA
GAAAATGTCATTACCCTGGACAATCCGGTCAAACGTGGTGAGCAGGTTATCGAACAGGTCA
CGCTGATGAAACCCAGTGCCGGGACGCTACGCGGTGTCAGTCTGGCTGCGGTTGCAAACTCC
GAAGTCGATGCACTGATTAAGGTGCTGCCGCGCATGACGGCACCGATGCTGACCGAGCAGG
AAGTCGCCGCGCTGGAACTGCCTGACCTTGTGGCGCTGGCCGGTAAGGTGGTCGGTTTTTTG
TCGCCGAACTCGGTGCAGTGACGTTTCCGAAAAATCTCTCGGTCGATGACCTGATGGCGGAT
GTGGCAGTGATATTTCACTGGCCGCCATCAGAACTGTATCCCATGAGCCTGACCGAACTCAT
CACATGGCGCGAAAAGGCGCTCCGGCGAAGCGGAAACACGAATGAGTAACAATGTAAAATT
ACAGGTATTGCTCAGGGCTGTTGACCAGGCATCCCGCCCGTTTAAATCCATCCGCACAGCGA
GCAAGTCGCTGTCGGGGGATATCCGGGAAACACAAAAATCACTGCGCGAGCTGAACGGTCA
CGCATCCCGTATTGAGGGATTCCGCAAGACCAGTGCACAGCTCGCCGTGACTGGTCATGCAC
TTGAAAAGGCACGGCAGGAGGCCGAAGCCCTTGCCACACAGTTTAAAAACACCGAACGTCC
GACCCGTGCTCAGGCGAAAGTCCTGGAATCCGCAAAGCGTGCGGCGGAGGACTTACAGGCG
AAATATAACCGCCTGACAGATTCCGTTAAACGCCAGCAGCGGGAACTGGCCGCTGTGGGAA
TTAATACCCGCAATCTTGCACATGATGAGCAGGGACTGAAAAACCGTATCAGTGAAACCAC
CGCACAGCTTAACCGTCAGCGTGATGCGCTGGTGCGTGTCAGTGCGCAACAGGCAAAACTTA
ACGCAGTAAAACAGCGTTATCAGGCCGGAAAGGAACTGGCCGGAAATATGGCCTCAGTGGG
CGCTGCCGGTGTGGGGATTGCGGCGGCGGGAACGATGGCCGGTGTTAAGCTACTGATGCCC
GGTTATGAGTTTGCGCAGAAAAACTCAGAATTACAGGCTGTGATCGGAGTGGCAAAAGACT
CCGCCGAAATGGCCGCACTCCGCAAGCAGGCGCGCCAGCTCGGCGACAATACCGCCGCCTC
GGCAGATGATGCAGCCGGTGCGCAGATTATTATTGCGAAAGCCGGTGGGGATGTTGATGCC
ATTCAGGCGGCAACGCCGGTCACGCTGAACATGGCGCTGGCGAACCGTCGCACAATGGAAG
AAAACGCCGCCCTGCTGATGGGGATGAAATCCGCCTTTCAGCTTTCAAACGATAAGGTCGCT
CATATCGGGGATGTTCTCTCCATGACGATGAACAAAACCGCCGCCGATTTTGACGGCATGAG
CGATGCGCTGACCTATGCCGCACCTGTGGCAAAAAATGCCGGTGTCAGCATTGAAGAAACC
GCCGCAATGGTCGGGGCGCTGCATGATGCAAAAATCACAGGCTCAATGGCGGGGACGGGAA
GCCGTGCCGTGTTAAGCCGCCTGCAGGCACCGACGGGAAAAGCATGGGATGCACTCAAAGA
GCTTGGAGTGAAAACCTCAGACAGCAAAGGAAACACCCGGCCAATATTTACCATTCTGAAA
GAAATGCAGGCCAGTTTTGAGAAAAACCGGCTCGGTACTGCCCAGCAGGCTGAATACATGA
AAACTATTTTCGGGGAGGAGGCCAGCTCAGCCGCTGCCGTGCTGATGACTGCCGCCTCAACC
GGAAAGCTGGACAAACTGACCGCTGCGTTTAAAGCCTCAGACGGGAAGACCGCCGAGCTGG
TAAATATCATGCAGGACAACCTAGGCGGTGACTTTAAAGCGTTTCAGTCCGCTTATGAGGCG
GTGGGGACTGACCTGTTTGACCAGCAGGAAGGCGCGCTGCGTAAGCTCACGCAGACGGCCA
CAAAGTATGTGTTAAAACTCGACGGCTGGATACAGAAAAACAAATCACTGGCGTCAACCAT
CGGCATCATTGCCGGCGGTGCACTGGCGCTTACTGGCATCATCGGTGCCATTGGCCTCGTAG
CCTGGCCGGTTATCACCGGCATCAATGCCATCATCGCGGCAGCAGGCGCAATGGGGGCAGT
CTTCACGACGGTTGGCAGTGCTGTTATGACCGCCATCGGGGCTATTAGCTGGCCGGTTGTGG
CCGTGGTGGCTGCCATTGTCGCCGGTGCGTTGCTTATCCGTAAATACTGGGAGCCTGTCAGC
GCATTCTTTGGTGGTGTGGTTGAAGGGCTGAAAGCGGCATTTGCGCCGGTGGGGGAACTGTT
CACGCCACTTAAACCGGTTTTTGACTGGCTGGGCGAAAAGTTACAGGCCGCGTGGCAGTGGT
TTAAAAACCTGATTGCCCCGGTCAAAGCCACCCAGGACACCCTGAACCGTTGCCGTGACACG
GGCGTCATGTTCGGGCAGGCACTGGCTGACGCGTTGATGCTGCCGCTTAATGCGTTCAACAA
ACTGCGCAGTGGTATTGACTGGGTACTGGAAAAACTCGGTGTTATCAACAAAGAGTCAGAC
ACACTTGACCAGACCGCCGCCAGAACTCATACCGCCACGTATGGTACCGGTGACTATATTCC
GGCGACCAGCTCTTATGCAGGCTATCAGGCTTATCAGCCGGTCACGGCACCGGCTGGCCGCT
CTTATGTAGACCAGAGTAAAAACGAATATCACATCAGCCTGACGGGGGGGACTGCGCCGGG
GACACAGCTTGACCGCCAGTTACAGGATGCGCTCGAAAAATACGAGCGGGATAAACGTGCG
CGCGCCCGTGCCAGCATGATGCATGACGGTTAAGGAGGTGACGAAAAATGATGCTCGCGTT
AGGTATGTTTGTTTTTATGCGCCAGACGCTGCCACACCAGACCATGCAGCGTGAATCAGATT
ATCGCTGGCCGTCAAATTCCCGTATCGGTAAACGGGATGCCTTTCAGTTTCTCGGTGTGGGT
GAGGAAAACATCACGCTGGCCGGTGTGCTTTATCCCGAACTGACCGGCGGCAAGCTGACGA
TGACCACGCTCAGGCTGATGGCAGAGGAGGGGCGGGCGTGGCCGTTGCTGGATGGCACCGG
CATGATTTACGGCATGTATGTCATCAGCAGGGTGAGTGAAACAGGGAGTATTTTCTTTGCAG
ACGGCACACCCCGGAAAATTGATTTTACGCTGTCACTCACCCGCGTTGATGAATCACTGGCC
GCGCTTTATGGCGATATCGGTAAACAGGCGGAATCGCTCATCGGTAAGGCCGGCAGTATGG
CGACCAGATTCACAGGTATGACGGGGGCGGGATAATGCTGGATGCGCTGACATTTGATGCA
GGCAGTACGCTGACGCCGGATTACATGCTGATGCTCGACAGCAGGGATATTACCGGCAATAT
CAGCGACCGTCTGATGAGCATGACCCTGACGGATAACCGGGGCTTTGAGGCTGACCAGCTTG
ATATTGAACTGAACGATGCCGACGGGCAGGTCGGGCTGCCGGTTCGTGGCGCTGTCCTGACG
GTGTATATCGGCTGGAAAGGTTTTGCCCTGGTATGCAAAGGGAAATTTACCGTTGATGAGGT
TGAACACCGGGGCGCACCGGATGTAGTCACCATCCGCGCCCGGAGTGCAGATTTTCGCGGG
ACGCTCAATTCCCGCCGGGAAGGCTCCTGGCATGACACCACGCTCGGTGCGATTGTTAAGGC
GATAGCCACCCGTAACAGGCTGGAAGCCAGTGTCGCTCCGTCACTGGCCGGAATAAAAATT
CCACACATCGACCAGTCGCAGGAGTCTGATGCGAAATTCCTGACCCGTCTTGCAGAACGCAA
CGGCGGTGAGGTGTCGGTAAAAATGGGAAAACTGTTGTTTCTCAAAGCGGGGCAGGGAGTG
ACGGCCAGCGGTAAAAAAATCCCGCAGGTCACCATAACCCGCAGCGACGGCGACCGCCATC
ATTTTGCGATTGCTGACCGTGGAGCCTACACCGGTGTAACGGCAAAATGGCTACACACTAAA
GACCCGAAGCCGCAAAAGCAGAAGGTAAAACTGAAACGCAAAAAGAAAGAGAAACACCTG
CGCGCACTGGAGCACCCGAAAGCGAAACCGGTCAGGCAGAAGAAAGCGCCTAAAGTACCG
GAAGCGCGTGAAGGTGAATACATGGCCGGTGAGGCTGACAACGTTTTTGCCCTGACCACGG
TATATGCCACGAAAGCGCAGGCCATGCGCGCCGCTCAGGCGAAGTGGGATAAACTGCAACG
GGGCGTTGCGGAGTTCTCTATCAGCCTGGCTACCGGTCGGGCAGATATTTACACGGAAACAC
CGGTCAAAGTGTCTGGCTTTAAGCGCGTCATAGACGAGCAGGACTGGACAATCACTAAGGT
GACACATTTTCTGAATAATAGCGGCTTCACGACGTCCTTAGAGCTTGAGGTCAGGCTTTCTG
ATGTGGAGTACGAAACAGAAGATGATGAGTGATGTTTTTGTTTTATCTGTTTGTTTTGTAAGG
ATAAATTAACTAAAATGGCACCATCAACAAAACCGGAAGAGGTGCTCGCGATGTTTCATTGT
CCTTTATGCCAGCATGCCGCACATGCGCGTACAAGTCGCTATATCACTGACACGACAAAAGA
GCGTTATCATCAGTGCCAGAACGTGAATTGCAGCGCCACGTTCATCACTTATGAGTCGGTAC
AGCGATACATCGTGAAGCCGGGAGAAGTCCACGCCGTAAGGCCGCACCCGTTGCCATCAGG
GCAGCAAATTATGTGGATGTAA

Minimal Genes to Include from a SaPI on a Vector or MGE.

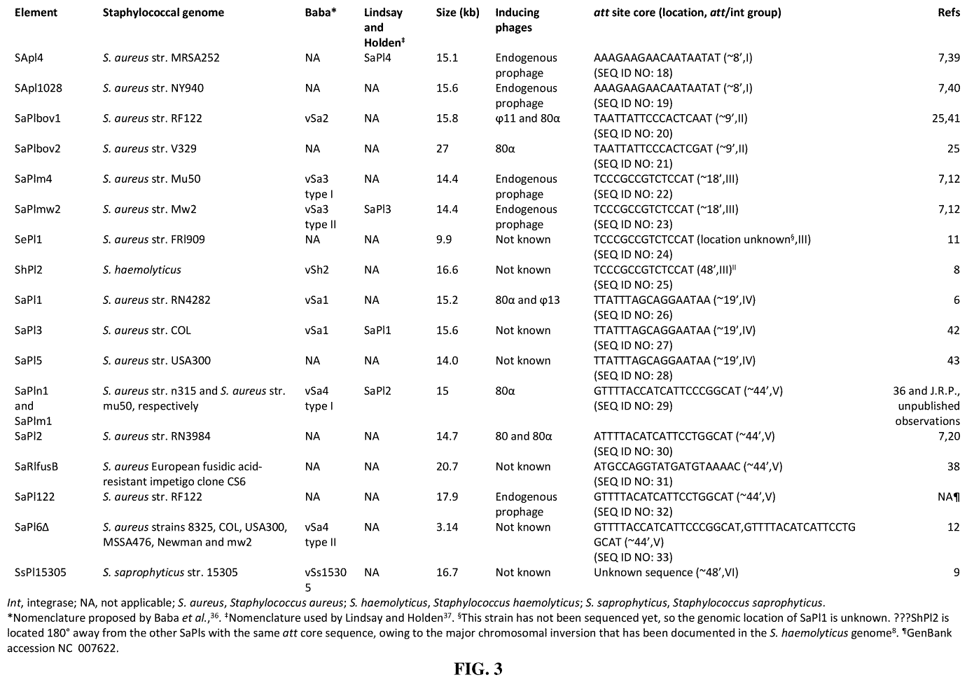

Several different SaPI systems exist. FIG. 2 is exemplified one of the well characterized SaPIs (SaPIbov1), which exploits phages phi11 or phi80alpha as helper phage. SaPIbov1 sequence (acc. number: AF217235.1)

### Packaging Signal

If one uses a defective helper phage with deleted packaging signal one can use that signal from the helper phage. In this example from S. aureus phi11 (acc. number: AF424781), as follows:

(SEQ ID NO: 11)
ANGATTTANTCC

For small capsid size (packages 15.8 kb instead of 43.6 kb), one can include cpmA and/or cpmB in the MGE or vector.

cpmA
(SEQ ID NO: 12)
MKTESYFKEYNQFVLDQHKAIQELEQERNALESKIKLDKSTYKQLIMDG
QDDKADNLYQATDADEKKLKALNKRLETKKSVSKEVKYQKTIELLKHQS
ELSSLYESEKQSAIEKLKKAVDAYNEIIDEIEDINDRYEDEHQQYASVY
SQEQLYDDKEARKALNGHFKENIFTSFINGNDLPYEHNNKLFLKC
cpmB (SEQ ID NO: 13):
MKTKYELNNTKKVANAFCLNEEDTNLLINAVDLDIKNNMQEISSELQQA
EQSKQKQYGTTLQNLAKQNRIIK

To activate helper phage phi11 one can include one, more or all of ptiA, B and M (provided separately in a host cell and not on the MGE or vector to be packaged)

ptiA
(SEQ ID NO: 14)
MDKQQIKDFVCDYHERTRSDVLIDDDINTDEFFSIADENSNEWMADDNI
DDHIVKNHLEMIVDRVANDKEFYIFDSLIQGRSYQDISGVLDCSEQSVR
FWYETLLDKIVEVIE
ptiB
(SEQ ID NO: 15)
MESIAEKETYHLPTEHLQVFNVIKNTSNKYITKTKILNQLGYEYNSSNE
RWLRRVINSLVYDYGYPIGCSYKPSERGYYIITTEQEKQQAMRSIKKLA
DGSMKRYEALKRIEV
ptiM (SEQ ID NO: 16):
MIAYPIRVGSVYRGEQMKLLKTKNCLYYRNGDNKLSEYQLLTQFNPTFI
NKKIRMCEFQIESMYHMSASTTTCDEMMGVVSVSYPIEKLVIKIIETKA
RLQNYKNRSISNMVLLKTVLNHYTEKEQKKVVKYMRSNGRYKPYNVIER
LQVDLYQASIKQRSERQKQRNIAIENSKIARVNAYHQSSYVKVV

Minimum Genes to Include in the Host Chromosome/Episome from Phi11.

Phi11 sequence (acc.number: AF424781)
gene #29 (terS) through gene #53 (lysin)
(SEQ ID NO: 17)
atgaacgaaaaacaaaagagattcgcagatgaatatataatgaatggatgtaatggtaaaaaagcagcaattacagcaggttata
gtaagaaaacagcagagtctttagcaagtcgattgttaagaaatgttaatgtttcggaatatattaaagaacgattagaacagatacaagaaga
gcgtttaatgagtattacagaagctttagcgttatctgcttctattgctagaggagaacctcaagaggcttacagtaagaaatatgaccatttaaa
cgatgaagtggaaaaagaggttacttacacaatcacaccaacttttgaagagcgtcagagatctattgaccacatactaaaagtacatggtgc
gtatatcgataaaaaagaaattactcagaagaatattgagattaatattggtgagtacgatgacgaaagttaaattaaactttaacaaaccgtct
aatgttttcaatagaaacatattcgaaatactaaccaattacgataacttcactgaagtacattacggtggaggttcgagcggtaagtctcacgg
cgttatacaaaaagttgtactcaaagcattgcaagattggaaatatcctaggcgtatactgtggcttagaaaagtacaatcaacaattaaagata
gtttgttcgaagatgttaaagattgtttgataaactttggtatttgggacatgtgcctttggaataagactgataacaaagttgaattgccaaacgg
cgcagtttttttgtttaaaggattagataacccagagaaaataaagtcgataaaaggcatatcagacatagtcatggaagaagcgtctgaattc
acactaaatgattacacgcaattaacgttgcgtttgagggagcgtaaacacgtgaataagcaaatatttttgatgtttaacccagtatctaaactg
aattgggtttataagtatttctttgaacatggtgaaccaatggaaaatgtcatgattagacaatctagttatcgagataataagtttcttgatgaaat
gacacgacaaaacttagagttgttagcaaatcgtaatccagcatattacaaaatttatgcgttaggtgaatttgctacactagacaaattggtttt
ccctaagtatgaaaaacgtttaataaataaagatgagttaagacatttaccttcttattttggattggactttggctacgttaatgatcctagtgcttt
tatacattctaaaatagatgtaaagaaaaagaagttatacatcattgaagagtatgttaaacaaggtatgctgaatgatgaaatagctaatgtcat
aaagcaacttggttatgctaaagaagaaattacagcagatagtgcagaacaaaaaagtatagctgaattaaggaatctagggcttaaaagga
ttttaccaaccaaaaaagggaagggctcggttgtacaagggttacaattcttaatgcaatttgaaatcattgttgatgaacgttgtttcaagacta
ttgaagagtttgacaactacacatggcaaaaggacaaagatacaggtgaatataccaatgaaccagtagatacatacaatcattgtatcgatt
cgttgcgttattcagtggaacgattctacagaccggttagaaaacgcacaaatctcagttcgaaagttgacacaataaaatctctaggattata
ggagggaacaaatgttaaaagtaaacgaatttgaaacagatacagatctacggggaaacataaattacttatttaatgatgaagccaatgttgt
ttacacatatgacgggacggaatccgatttattacaaaacgttaatgaagtaagtaaatacattgaacatcacatggattaccaacgacctaga
ttgaaagtgttaagtgattattacgaaggtaaaactaagaacttagttgagttaacacgacgcaaagaagagtacatggcagataaccgtgta
gcgcatgattacgcatcttatattagcgattttatcaacggctatttcttgggtaatccaattcaatatcaagatgatgacaaagatgtattagaag
ttattgaggcgttcaatgatttaaatgatgttgagtcacacaatagatctttaggattagatttgtcaatttatggcaaagcttatgagttaatgatta
gaaaccaagatgatgaaacgcgtttatacaagagtgatgcaatgagtacttttgtcatatacgacaatacaattgaacgtaatagtatcgcagg
cgttagatatttaagaactaaaccaatagacaagactgacgaagatgaagtgtttacagttgatttattcacttcacacggtgtttatagatatctt
accagtagaacaaatggattgaagctcacaccacgtgaaaacggttttgaatcacactctttcgaacgtatgcctattacagaatttagcaaca
acgaaagaagaaaaggggattatgagaaagtaatcactttaattgatttgtatgataatgctgaatcagatactgctaactatatgagtgatttaa
atgacgctatgttacttattaaaggtaatttaaatttagatcctgtagaagttagaaaacaaaaggaagctaacgtgttgtttttagaaccgactgt
ttatgctgatagcgaaggtagagaaacagaaggctctgttgatggtggttatatttataagcaatacgatgtacaaggtaccgaagcttataaa
gaccgtttaaacagtgatatacacatgtttaccaacacgcctaacatgaaagatgataactttagcggcactcaatcgggcgaggcaatgaa
atacaaattatttggattggaacaacgtactaaaactaaagaaggattgtttactaaagggttaagacgtcgtgctaagttgttagagacaatac
ttaaaaatacatggtcgattgacgctaacaaagatttcaatactgttagatacgtatacaacagaaacttacctaaatcattgattgaagaattaa
aagcttatattgattctggtgggaagattagccaaacaactttaatgtctctattctcgttcttccaagaccctgaattagaagttaagaaaatcga
agaagatgagaaagaatctattaaaaaagctcaaaaaggtatttataaagaccctagagacatcaatgatgacgaacaagatgatgatacaa
aagatactgttgataaaaaggaatgattgtaattgcctaacaaaaacactcaagaatattgggaagaacgcggacgcaaagcaatcgagaat
gagttgaagcgtgataaaactaaagctgaagaaatagaacgtatattgaatatgatgattaagcgcattgaaaaagagatcaatgcgtttattg
tcaagtacggagattttgcaggcgttacattacaagaagcacaaaagattattgatgagttcgatgtaaaagcgtttcaagaagaagcaaaaa
gattggtcgaaaacaaggagtttagcgatagagcaaatgaagaattaaagaagtataacacgaaaatgtatgtatctagagaacagatgtta
aagattcaaatagaattcttaattgcttatgcaacagctcaaacagaattatcgatgagggaatatttcgaatcaacagcttatcgtgtgttcagt
gatcaagcgggtattttaggtgaaggtgtacaagtagctaaagaagttatagatacaatcgttgatacacaatttcatggtgtcgtttggtcaga
gcgattatggactaataccgaagcaatgaaacaagaagtagaagaaataattgctaatgtagttattagaggtcgacatcctaatgaatatgtt
aaagatatgcgcaagcacttaaataaattcgaaggcacagcacgacaaaagaccgcagcaattaaatcattgctttatacggaatcggcac
gtgttcacgcacaatcaagcattgacagcatgaaagaaatttcaccggaaggatattatatgtatattgcaaaaatcgataatagaacaactaa
agtatgcaaagggcttaatggagaaatattcaaagttaaagacgctaaaattggtgttaatttctatcctatgcatatcaattgtcgttcagattgc
gctttactacctaaatctatgtggccgaaaaaaccaagcaagaaacgaaaaacaaaatacttcggagggaaagtgaaaagcggtgattgatt
taaaagtgaagttttttaaaggcaagttagttttgtatgacagtaaattaaatgtttggaggatactaatatgagtaatactgacaaataccttaga
gacatagcaagagaattaaaaggtatacgtaaagagttacaaaagcgaaacgaaacagttattattgatgcaaacttagacagtttaaggtcg
gcagtattagccgataaagaaaaatcgaaatataatgaacctctcttttaatagctagcacttaattgtgttggctattttttatgtccaaaacgtgc
tgatgacataaaaagcacgcatggaaaaacagtcgacagactataaatggaggtatatctcatggaagaaaataaacttaagtttaatttgca
attttttgcagaccaatcagatgatccggacgaaccaggcggagatggtaaaaaaggaaatcctgataagaaagaaaatgacgaaggtact
gaaataactttcacgccagagcaacaaaagaaagttgatgaaatacttgaacgtcgtgtagcccacgaaaagaaaaaagctgatgagtatg
caaaagaaaaagcagcagaagctgctaaagaagctgctaaattagcgaaaatgaacaaggatcaaaaagatgaatatgaacgcgaacaa
atggaaaaagaactggaacaattacgttcagaaaaacaattaaacgaaatgcgttcagaagcacgaaaaatgttgagtgaagcggaagttg
attcatcagatgaggttgtcaatttagttgtaacagatactgctgaacaaactaaattgaatgttgaagctttttctaatgcagtaaaaaaagcggt
taatgaagcggttaaggttaacgctagacaatcgccattgactggtggagattcatttaatcactcgactaaaaataaaccgcaaaacttagct
gaaatagctagacaaaaaagaattattaaaaattaacggaggcatttaaatggaacaaacacaaaaattaaaattaaatttgcaacattttgca
agtaacaatgttaaaccacaagtatttaaccctgacaatgtaatgatgcatgaaaagaaagatggcacgttgttaaacgactttacaacaccta
tcttacaagaggttatggaaaactctaaaatcatgcaattaggtaagtacgaaccaatggaaggtactgagaagaagtttactttttgggctgat
aaaccaggtgcttactgggtaggtgaaggtcaaaaaatcgaaacgtctaaggctacttgggttaatgctacaatgagagcgtttaaattaggg
gttatcttaccagtaacaaaagaattcttgaattacacttattcacaattctttgaagaaatgaaacctatgattgctgaagctttctataaaaagttt
gacgaggcaggtattttgaatcaaggtaacaatccgttcggtaaatcaattgcacaatcaattgaaaaaactaataaggttattaaaggtgactt
cacacaagataacattattgatttagaggcattgcttgaagatgacgaattagaagcaaatgcatttatctcaaaaacacaaaacagaagcttg
ttacgtaaaattgtagatcctgaaacgaaagaacgtatttatgaccgtaacagtgattcgttagacggtctacctgtggttaaccttaaatcaag
caacttaaaacgtggtgaattaatcactggtgacttcgacaaattgatttatggtatccctcaattaatcgaatacaaaatcgatgaaactgcaca
attatctacagttaaaaacgaagatggcacacctgtaaacttgtttgaacaagacatggtggcattacgtgcaactatgcatgtagcattgcata
ttgctgatgataaagcgtttgctaagttagttcctgctgacaaaagaacagattcagttccaggagaagtttaataaataattaggagtggtaac
atgcccgaaatcattggaattgttaaagtagattttacagatttagaagataacagacatgtctatatgaaagggcatgtctaccctcgtaaagg
ttataatcctacagatgaacgtatcaaagctttagctagtgttgaaaataaacgcaacaaacaaatgatttacattgtaaatgacaaattaaccaa
aaaagaacttgtcgaaatagcaagtgttgctggcttacaagttgatgaaaaacaaacaaaagctgaaattatcaatgcttttgagtcactagag
taggtggttatatgactacgctagctgatgtaaaaaaacgtattggtcttaaagatgaaaagcaagatgaacaattagaagaaatcataaaaa
gttgtgaaagccagttgttatcaatgttacctattgaagttgaacaaataccggaaaggtttagttacatgattaaagaagttgcagttaaacgct
acaacaggattggtgctgaaggtatgacatcagaagcggttgacggacgtagcaatgcgtatgaattgaacgatttcaaggagtatgaagct
attattgataattactttaatgctagaacgagaactaaaaaaggaagggctgtgttcttttgagatatgaagatagagttatttttcaattagaaca
agtagcaacttacaatcctaaaactagcaaaaaagaaaacacactaatcacttatgatgcgataccatgcaatattaaccccatttctagagca
agaaagcaacttgaatttggtgatgtaaaaaacgatgtaagtgttctgaggataaaagaatcaatatcttaccctgttagccacgtgttggttaat
ggcattcgctacaagatagttgatacaaggatatacagacacgaaacgtcatattatatcgaagaggtcaattgatgaatatagatggattaga
cgcactgttaaaccaatttcacgatatgaaaaccaacattgatgatgatgtagatgatattttacaggaaaacgccaaagaatatgtagtacga
gctaaattgaaagctagagaagtaatgaataagggttattggactggtaatttatcacgcaatatcagatataaaaaaactggcgatttgcaata
cactatcacatcgcacgcagcttatagtggtttcttagaatttggtactcgatacatggaggctgaaccttttatgtggccggtatacgaagtgat
taggaaatcaactgtagaagaattgaaagcgttgtttgaataggagataaaagcatgacaccgaacttacaactttataataaagcgtatgaaa
cgctacaaggatatggattccctgttatttctcgtaaagagatgcaacaagagattccgtatcctttttttgtaataaaaatgccggagtcaaata
gaagtaagtacacgtttgatagttattctggcgatacgaatttagttattgatatttggagtgtaagcgatgatttaggacatcatgacggacttgt
taaaagatgtattgatgatttaacacctagcgttaaaacaaacgattatgactttgaagaagaagatactaacatcacacagttagttgatgata
ctaccaatcaagaattgctacacacatcagtaacgatatcttacaaaacattttaaaaaacggaggaatattgaatggcaaatatgaaaaatag
taatgatcgtattattttatttagaaaagctggcgaaaaagtagatgctactaaaatgctttttttaactgaatacggcttatcacatgaagctgata
cagatacagaggatacaatggacggttcttataacactggtggttctgttgagtcaacaatgtctggtactgctaaaatgttttatggtgacgatt
ttgcagatgaaattgaagatgcagttgtagatcgcgtattgtatgaagcttgggaagttgaaagtagaataccaggcaaaaatggggattccg
ctaaatttaaagcgaaatatttccaaggtttccacaataaatttgaattaaaagcagaagctaacggtattgatgaatatgaatatgaatatggag
tgaatggtcgtttccaacgtggatttgcaacactacctgaggctgtaacaaagaaacttaaggcgactggatacagattccacgacactacaa
aagcagatgcattaactggcgaagatttaacagcaattccacaacctaaagtagattcaccaccggttgcaccaagagaggtataaaaatag
ggcgttaagccctttttattttgtttaaattaattatgaatggagattttaagttatgaatgtagaaattaacggaaagtcattagaattaagttttggt
tttaaatttttaagagaaatcgataaccgattaggtttaaaagttgaacaagcttctatcggtcaaggtgtatcaatgttgcctgtaggtttagaaag
tggaaatccggttgtgattggcgaagttttaatcgcagctacatctcacttaaaaaaacaagcaattactattaataacattgatgaagcattaga
tgaaatcgcagaaaatatcggactagaagaattcggttcggatattttaacggagttgggaaagcgacctatgacccgaaacctagtcgaag
tagtggaaacggaagaaaaaccagcggaagcctaataacttacgacagaatcgttataacttgtatgtcaacacttggtattacagatttgaac
gttattgagcaaatgacattaacagaatataactatcgaatgtatgcgaaagagtatgaaatgctaacccaagaattcgaacgttacaaacttg
cgtttgctattcgtgatgctgcagctactaaaaatgttgggacagaaaataaacctaaagaggaatatgtttttaacaatgcaaacgacgtattg
ccttatgaagaaaatatccaacggcttaacgaaggtaaagatataagatttagtagcgaacgtgatgaatacgaaccacaaaataatgaattc
tttaaagttatagcagaatttaataagcaatagaaagagaggtgttaatgtgacggaatataaaattaaagcgactattgaagctagtgtagcc
aaattcaaaaggcaaattgatagtgcggttaagtctgtgcaaagatttaaacgagtagcagatcaaactaaagatgttgaattaaacgctaac
gataaaaatttacaaaaaactatcaaagttgctaaaaagtctttagatgcctttagtaacaaaaatgtaaaagctaaattagatgctagtatacaa
gatttacaacaaaaggtactagaatcgaattttgaactagacaaactaaactctaaagaagttacaccagaagttaagttgcaaaaacaaaag
ttgattaaagatatcgctgaaacagaagctaaattatcagaattagaaaagaaacgtgtcaatattgacgtcaatgcagataacagtaaattca
atcgagtgttaaaagtatctaaagctagtctcgaagcattaaataggtctaaagccaaagctattatagacgtggacaatggtgttgctaactct
aaaatcaaacgtactaaagaagaacttaaaagtattccgaacaaaactagatctcgacttgatgtagatacagggctttctataccaactattta
tgcgtttaaaaaatcattagacgcattgccgaacaaaaaaacaacaaaggtagatgtcgatactaatggtttaaagaaagcttatgcctacata
ataaaagcaaatgacaattttcaaagacagatggggaatttagctaatatgttccgtgtgttcggtactgtaggttctaatatggttggtggatta
cttacatcatcttttagtatcttaatacctgtaatagcgagcgtagtacctgtagtatttgcgctattaaacgctatcaaagtgttaactggtggtgt
acttgctttaggtggtgccgtagcaatagcgggagcaggatttgtagcgtttggcgcaatggctatcagcgctataaagatgcttaatgatgg
cactttacaagctagctcagcaacaaacgaatacaaaaaagcgttagatggcgtaaagtcagcatggactgatattataaagcaaaatcaat
ccgctatcttcacaactcttgcaaatggtttaaatactgttaaaactgcaatgcagagcttacaaccgttttttagtggtatttcaagaggaatgga
agaagcgtctcaaagcgtgcttaaatgggctgaaaatagcagtgtagcttcaagattctttaatatgatgaatacaacgggtgtttcggtattta
acaagctattaagtgctgcaggtggttttggtgacggattagtcaatgtattcacgcaattagcaccactgtttcaatggtcggctgattggttgg
atagattaggtcaatctttctctaactgggctaatagtgcagctggagaaaattcgataactcgttttattgaatacacaaaaacaaacttacctat
cattggtaatattttcaaaaatgttttcgttggaattaacaatttgatgaatgcattcagcggatcatcaactggcatattccaatctcttgaacaaat
gacagctaagtttagggaatggtctgaacaagtaggacaatctcaagggtttaaagactttgtcagttatatacaaacaaatggaccactaata
atgcaattgattggaaacatcgcaagaggattagttgcattcgcaacagcaatggctcctatagctagtgcagtattacgcgttgcagttgcaa
taactggttggatagctaacttgtttgaggcgcatccagctacagcacaattagttggtgtcattataactttagttggtgcatttagatttttaatac
cgattattcttgctgtatctaactttatgggtggcggattaataggtagaatcattgcattagtaagtaagttcggtttattaagagcgggattaac
aattttaaaaggtgcgttcatgttattaaaaggaccattaaaaattatatcagttatattccaattgttattcggtaagattggattaattagaaatgct
atcacaggactagtaactgtgtttggtattttaggcggtccaataacaatagtaattggtgtaattgctgcattaatagctatattcgttttattgtgg
aataaaaatgaaggattcagaaactttattataaatgcttggaatgcgataaaaacgtttatggttaatgtttggaatgtattaaaagctgtagctt
cggttgtatggaatgctattttaacagctatcactacagcagtatcgaatgtttacaattttataatgattgtttggaatcaaatagtcgcttatttac
aagggctatggaatggaattatcgctattgcaacaacagtatggaaccttttagttacaatcattacaactgttttcacgacgataatgacaatag
ttatgacgatatggacagctatttggacgttcttaagtacaatctggaatacgataattacaatcgctactacgatttggaatttgttagtcactgta
ataactacagtgtttaccacaattatgactatcgcaataacaatttggaacgctatttggacgttcttacaaacgttgtggaacactatagttactg
tggcaactaaggtttggaacgctatcactacagctatatctactgcgttacaagcggcatggagttttatttctaatatatggaatacgatttgga
gtttcttatctggtatattaacgacaatttggaataaagttgtaagcatattcacacaagttgtttcaactatatcagacaaaatgtctcaagcttgg
aacttcattgtcactaaaggtatgcaatgggtatctactataacaagtacgctaattaactttgttaatagagttgttcaaggattcgttaatgttgta
aacaaagttagtcaaggtatgacaaatgcagtaaataaagttaaaagctttgtggatgactttgtatcagcaggtgctgatatgatccgtggttt
gatgagaggtattggtaatatggctagagacttagctgaaaaagcagctagtgtagcaaaaggtgctttaaatgcagccaaaagagcgcta
ggtattcactcaccttcacgtgaattcatggatgttggtatgtattcaatgttaggtttcgttaaaggtatagataatcattcaagtaaagttatccgt
aatgtttctaatgttgcagataaagtagttgatgcatttcaacctacattaaacgcacctgacatttctagtattacaggaaacttaagtaatttagg
tggaaatataaatgcgcaagtacaacacacacattctattgaaacatcaccgaacatgaaaactgttaaagttgaattcgatgtcaataacgat
gcgcttactagtattgttaacggcagaaatgctaaacgcaattctgagtattacttataaaggaggttacaaatggacatagaattaacaaaaa
aagatggtactgtaatcaaattaagtgaatacgggtttatcgttaacgatatagtaattgatagcatgcaaatcaacacaaagtatcaagacaaa
gaaaatatgaacggtcgtatattaatggggagcaattatatcagtagagatatagttgttccttgtttttgtaaagttaaaaatcgttcagacattg
cttatatgcgagatatgttgtattcgttaacgacagacatagaacctatgtatttgcgagaaatcagaagaaaagaagagttgaattacaggttt
actcaaccaacttctgatgattacgtgaaattagataaaaacaacttcccggattacgaatattcaagacacgatcaacaaatttatgtaaatgg
taaacagtataaagttatttttaacggagttataaaccctaaacaaaaaggtaataaagtttcttttgaactaaaattcgaaactacagaattacca
tacggtgaaagtattggaacaagcctagagttagaagaaaacaaaaaggttggattgtggtcgtttgattttaatattgattggcatgcaggcg
gagacaaaagaaagtatacatttgaaaatttgagcaaaggtacagtttactatcatggtagtgctcctaacgaccaattcaacatgtataaaaa
gataacaattattttaggcgaagatacagaatcgtttgtatggaatttaacgcatgctgaaataatgaaaatcgaagggatcaaactaaaagct
ggagacagaattgtttatgatagcttccgagtttataaaaacggtgttgaaataagtaccgaaacgaatatagcccaaccaaaatttaaatacg
gagctaataaatttgagtttaatcaaacggtacaaaaagttcagtttgatttgaaattttattataagtaggtgtcagaatgacaataactattaaac
cacctaaaggtaatggcgcacctgtaccagtagaaacaactttagtaaaaaaagttaatgctgacggtgtattaacttttgatattctagaaaat
aaatatacttatgaagttattaacgctatagggaaaagatggattgttagtcatgtcgaaggtgaaaacgacaagaaagaatatgtaataactg
tcattgataggaaatcagaaggcgacagacaactggttgaatgtactgctagagagattcccatagacaagttaatgattgatagaatttatgtt
aatgtaacaggatcttttacagtagaaagatattttaacattgtgtttcaaggtactggaatgctttttgaagtcgagggcaaagttaaatcttcaa
agtttgaaaatggtggtgaaggcgatacaaggttagaaatgtttaaaaagggattagaacatttcggtttagaatataaaataacgtatgacaa
aaagaaagacagatataagtttgtattgacgccttttgcaaatcaaaaagcgtcttattttatttctgacgaagtcaacgccaacgctataaaact
cgaggaagatgcaagtgatttcgccaccttcattagaggatatggtaattattcaggagaagaaacattcgaacacgctgggctcgtaatgg
aagctagaagtgcattagctgaaatatacggcgacatccacgcagaaccatttaaagatggtaaagtgactgaccaagaaactatggataaa
gaattacaatcgagattgaaaaagtcgttaaaacaatctttgtctttggactttttggtgttaagagaatcatatccagaagcagacccacaacc
cggagacatagtacaaataaaatctaccaaactaggtttgaatgatttagtccgtatagtacaagttaaaacgattaggggtataaacaatgta
attgttaagcaagatgtaacgcttggtgagtttaatcgagaacaacgatatatgaaaaaagttaatactgcagctaactatgtttctggattaaat
gatgttaacctttctaatcctagtaaagcggcagaaaacttgaagtctaaagtagcgtcaatagctaaatcaacactcgatttgatgagtagaa
ctgatttgattgaagataaacaacagaaggtaagctctaaaactgtgactacatctgacggcactatcgttcatgattttatagataaatcaaac
attaaagatgtaaaaacgattggaacgattggcgattctgtagctagaggatcacatgcgaaaactaatttcacagaaatgttaggcaagaag
ttaaaagctaaaacgaccaaccttgcaagaggtggcgcaacaatggcaacagttccaataggtaaagaagcggtagaaaacagcatttata
gacaagcagagcaaataagaggagacctaatcatattacaaggtacagatgatgactggttacatggttattgggcaggcgtaccgatagg
cactgataaaaccgacactaaaacgttttacggcgccttttgttctgcaattgaagttatcaggaaaaataatccagcttcaaaaatacttgtaat
gacagctactaggcaatgccctatgagtggtacaacgatacgccgtaaagatacggacaaaaacaaactagggttaactttagaggattatg
tcaatgctcagatattggcttgtagtgaattggatgtaccagtatatgatgcttatcacacagattatttcaaaccatataatccagcatttagaaa
atctagcatgcctgatggattacatcctaatgaaagaggtcatgaagttattatgtatgagcttattaaaaattattatcagttttatggatagtaaa
ggaggaaaacatgagtaataaactaattacagatttaagtagagtctttgactacagatatgtagatgaaaatgagtataactttaaacttatttc
agacatgctgacggattttaatttctctcttgaataccacagaaataaagaggtattcgcacatgatggagaacaaataaagtatgaacatttaa
atgttacaagtaacgtctctgactttttaacatatttaaacggtcgatttagcaacatggtactaggtcataacggcgacggtatcaacgaagta
aaagacgcgcgcgttgataatacaggttatggtcataagacattgcaagatcgtttgtatcatgattattcaacactagatgttttcactaaaaag
gttgagaaagctgtagatgaacactataaagaatatcgagcgacagaataccgattcgaaccaaaagagcaagaaccggaatttatcactg
atttatcgccatatacaaatgcagtaatgcaatcattttgggtagaccctagaacgaaaattatttatatgacgcaagctcgtccaggtaatcatt
acatgttatctagattgaagcccaacggacaatttattgatagattgcttgttaaaaacggcggtcacggtacacacaatgcgtatagatacatt
gatggagaattatggatttattcagctgtattggacagtaacaaaaacaacaagtttgtacgtttccaatatagaactggagaaataacttatggt
aatgaaatgcaagatgtcatgccgaatatatttaacgacagatatacgtcagcgatttataatccggtagaaaatttaatgatttttagacgtgaa
tataaacccactgaaagacaacttaagaattcgttgaactttgttgaggttagaagtgctgacgatattgataaaggtatagacaaagtattgtat
caaatggatatacctatggaatacacttcagatacacaacctatgcaaggtatcacttatgatgcaggtatcttatattggtatacaggtgattcg
aatacagccaaccctaactacttacaaggcttcgatatcaaaacgaaagaattgttatttaaacgtcgcatcgatataggcggtgtgaataaca
actttaaaggagatttccaagaggctgagggtctagatatgtattacgatctagaaacaggacgtaaagcacttctaatcggggtaactattgg
acctggtaacaacagacatcattcaatttattctatcggtcaaagaggtgtaaaccaattcttgaaaaacatcgcacctcaagtatcaatgactg
attcaggcggacgtgttaaaccgttaccaatacagaacccagcatatctaagtgatattacggaagttggtcattactatatctatacgcaaga
cacacaaaatgcattagatttcccgttaccgaaagcgtttagagatgcagggtggttcttggatgtactgcctggacactataatggtgctctaa
gacaagtacttaccagaaacagcacaggtagaaatatgcttaaattcgaacgtgtcattgacattttcaataagaaaaacaacggagcatgga
atttctgcccgcaaaacgccggttattgggaacatatccctaagagtattacaaaattatcagatttaaaaatcgttggtttagatttctatatcact
actgaagaatcaaaccgatttactgattttcctaaagactttaaaggtattgcaggttggatattagaagtaaaatcgaatacaccaggtaatac
aacacaagtattaagacgtaataacttcccgtctgcacatcaatttttagttagaaactttggtactggtggcgttggtaaatggagtttattcgaa
ggaaaggtggttgaataatggtagtagataatttttcgaaagatgataacttaatcgagttacaaacaacatcacaatataatccggttattgac
acaaacatcagtttctatgaatcagatagaggaactggtgttttaaattttgcagtaactaagaataacagacccttatctataagttctgaacatg
ttaaaacatctatcgtgttaaaaaccgatgattataacgtagatagaggcgcttatatttcagacgaattaacgatagtagacgcaattaatggg
cgtttgcagtatgtgataccgaatgaatttttaaaacattcaggcaaggtgcatgctcaggcattctttacacaaaacgggagtaataatgttgtt
gttgaacgtcaatttagcttcaatattgaaaatgatttagttagtgggtttgatggtataacaaagcttgtttatatcaaatctattcaagatactatc
gaagctgtcggtaaagattttaaccaattaaagcaaaatatggctgatacacaaacgttaatagcaaaagtgaatgatagtgcgacaaaagg
cattcaacaaatcgaaatcaagcaaaacgaagctatacaagctattactgcgacgcaaactagtgcaacacaagctgttacagctgaattcg
ataaaatagttgaaaaagagcaagcgatttttgaacgtgttaacgaagttgaacaacaaatcaatggcgctgaccttgttaaaggtaattcaac
aacgaattggcaaaagtctaaacttacagatgattacggtaaagcaattgaatcgtatgagcagtccatagatagcgttttaagcgcagttaac
acatctaggattattcatattactaatgcaacagatgcgccagaaaagacggatataggcacgttagagaagcctggacaagatggtgttgat
gacggttcttcgttcgatgaatcaacttatacatcaagcaaatctggtgtgttagttgtttatgttgttgataataatactgctcgtgcaacatggta
cccagacgattcaaacgatgagtacacaaaatacaaaatctacggcacatggtacccgttttataaaaagaatgatggaaacttaactaagca
atttgttgaagaaacgtctaacaacgctttaaatcaagctaagcagtatgtagatgataaattcggaacaacgagctggcaacaacataagat
gacagaggcgaatggtcaatcaattcaagttaacttaaataatgcgcaaggcgatttgggatatttaactgctggtaattactatgcaacaaga
gtgccggatttaccaggtagcgttgaaagttatgagggttatttatcggtattcgttaaagatgatacaaacaagctatttaacttcacaccttata
actctaaaaagatttacacacgatcaatcacaaacggcagacttgagcaacagtggacagttcctaatgaacataaatcaacggtattgttcg
acggtggcgcaaatggtgtaggtacaacaatcaatctaactgaaccgtacacaaactattctattttgttggtaagtggaacttatccaggtgg
cgttattgagggattcggactaaccgcattacctaacgcgattcaattgagtaaagcgaatgtagttgactcagacggcaacggtggcggtat
ttatgagtgcttactatccaaaacaagtagcactactttaagaatagataacgatgtgtactttgatttaggtaaaacatcaggttctggagcgaa
tgccaacaaagttactataactaaaattatggggtggaaataatgaaaatcacagtaaacgataaaaacgaagttatcggattcgttaatactg
gcggtttacgcaatagtttagatgtagatgataacaatgtgcctattaaatttaaagaagagttcgaacctagaaagtttgttttcactaacggcg
aaattaaatacaatagcaatttcgaaaaagaagacgtaccgaatgcatcaaaccaacaaagtgcgtcagatttaagtgatgaggaacttcgc
ggaatggttgcgagtatgcaaatgcaggtggcacaagtaaacgtattaacaatggaattagctcaacaaaacgctatgttaacacaacagtt
gactgaactgaaaactaacaaaacaagtactgagggggacgtttaaataatgaagatgatttatccaacttttaaagacattaaaactttttatgt
ttggggttactataaaaacgagcaaattaagtggtacgtagacaagggtttaatcgataaagaagaatacgctttaatcactggagaaaaatat
ccagaaacaaaagatgaaaagtcacaggtgtaatgcttgtggctttttaatttgaataaagtgggtggcataatgtttggatttaccaaacgaca
tgaacaagattggcgtttaacgcgattagaagaaaatgataagactatgtttgaaaaattcgacagaatagaagatagtcttagagcgcaaga
aaagatttatgacaaattagatagaaattttgaagaattaaagcgcgacaaggtagaagatgaaaagaataaagaaaagaatgccaagaata
ttagagacataaaaatgtggattctaggtttgatagggactatcttcagtacgattgtcatagctttactaagaactgtttttggtatttaaaggagg
tgattaccatgcttaaagggattttaggatatagcttctgggcgtgcttctggtttggtaaatgtaaataacagttaagagtcagtgcttcggcact
ggctttttattttgattgaaatgaggtgcatacatgggattacctaatccgaaaaatagaaagcccacagctagtgaagtggttgaatgggcgtt
atatatcgctaaaaacaaaatagctattgatgtacctggttctggaatgggagcacaatgctgggatttacctaattatttactcgataaatattgg
gggtttagaacatggggaaatgctgatgctatggctcaaaaatccaattatagaggtagagatttcaagataattagaaatacaaaagattttgt
accacaaccaggcgactggggtgtttggactggtggttgggcaggacatgtaaacattgtagtgggaccatgcacaaaagactattggtat
ggcgtagatcaaaactggtatacaaataacgcaacaggaagtccaccttataaaattaaacactcttatcatgatggaccaggtggaggggtt
aaatattttgttagaccaccatatcatccagacaaaactacaccggcacctaaaccagaagatgatagtgatgataacgaaaaaaataataaa
aaagttccaatttggaaagatgtaacaactataaagtacactatttctagccaagaggttaattatccagaatatatttatcactttatagtagaag
gtaatcgacgactcgaaaaacctaaaggaataatgattagaaacgcacaaacgatgagctcggtagaaagtttatataacagtaggaagaa
atacaaacaggatgtagaatatccccacttttatgttgatagacataatatttgggcacctagaagagctgtatttgaagttcctaatgaacctga
ttatatagttatagacgtatgtgaagattatagtgcgagtaaaaatgaatttatttttaatgagattcacgcaatggttgtagctgtagatatgatgg
ccaaatatgagatacctctaagtattgaaaatttaaaagtagacgacagcatttggcgttcaatgttggaacatgttaattggaatatgattgaca
acggtgttcctcctaaagataaatacgaagcattagaaaaggcattacttaatatatttaaaaacagagaaaaattattaaattctataactaagc
caacagtaacaaaatctagaataaaagttatggtagataataaaaacgctgatatagctaatgtaagagactcgtcaccaacagccaacaatg
gttcggcatctaaacaaccgcagatcataacagaaacgagtccttatacattcaaacaagcactggataaacaaatggcaagaggtaaccc
gaaaaaatctaatgcttggggttgggctaacgctacacgagcacaaacgagttcagcaatgaatgtaaagcgtatatgggaaagtaacaca
caatgctaccaaatgcttaatttaggcaagtatcaaggtgtttcagttagcgcacttaataagatacttaaaggtaagggaacattgaataatca
aggtaaagcgttcgcagaagcttgtaaaaagcacaacattaatgaaatttatttaatcgcgcatgctttcttagaaagtggatatggaacaagta
acttcgctaacggaaaagatggagtatacaactacttcggcattggcgcttacgacaacaatcctaactacgcaatgacgtttgcaaggaata
aaggttggacatctccagcaaaagcaatcatgggcggtgctagcttcgtaagaaaggattacatcaataaaggtcaaaacacattgtaccga
attagatggaatcctaagaatccagctacccaccaatacgctactgctatagagtggtgccaacatcaagcaagtacaatcgctaagttatata
aacaaatcggcttaaaaggtatctacttcacaagggataaatataaataaagaggtgtgtaaatgtacaaaataaaagatgttgaaacgagaa
taaaaaatgatggtgttgacttaggtgacattggctgtcgattttacactgaagatgaaaatacagcatctataagaataggtatcaatgacaaa
caaggtcgtatcgatctaaaagcacatggcttaacacctagattacatttgtttatggaagatggctctatattcaaaaatgagccccttattatc
gacgatgttgtaaaaggtttccttacctacaagatacctaaaaaggttatcaaacacgctggttatgttcgctgtaagctgtttttagagaaagaa
gaagaaaaaatacatgtcgcaaacttttctttcaatatcgttgatagtggtattgaatctgctgtagcaaaagaaatcgatgttaaattggtagat
gatgctattacgagaattttaaaagataacgcgacagatttattgagcaaagactttaaagagaaaatagataaagatgtcatttcttacatcga
aaagaatgaaagtagatttaaaggtgcgaaaggtgataaaggtgaaccgggacaacctggagcaaaaggtgaagcaggtaaaaaagga
gaacaaggcgcacccggtaaaaacggtactgtagtatcaatcaatcctgacactaaaatgtggcaaattgatggtaaagatacagatatcaa
agcagaacctgagttattggacaaaatcaatatcgcaaatgttgaagggttagaaaataaattgcaagaagttgaaaaaatcaaagatacaac
tctcaacgactctaaaacgtatacggatacaaaaattgctgaactagttgatagcgcgcctgaatctatgaacacattaagagaattagcaga
agcaatacaaaacaactctatttcagaaagtgtattgcaacagattggctcaaaagttaatacagaagattttgaggaattcaaacaaacacta
aatgatttatatgctccaaaaaatcataatcatgacgagcggtatgttttgtcatctcaagcttttactaaacaacaagcggataatttatatcaact
aaaaagcgcatctcaaccgacggttaaaatttggacaggaacagaaaatgaatataactatatatatcaaaaagacccgaatacgttatattta
attaaagggtgatttttatggaaggtaattttaaaaatgtaaagaagtttatttacgaaggtgaagaatatacaaaagtatatgctggaaatatcc
aagtatggaaaaagccttcatcttttgtaataaaacccttacctaaaaataaatatccggatagcatagaagaatcaacagcaaaatggacaat
aaatggagttgaacccaataaaagttatcaggtgacaatagaaaatgtacgtagcggtataatgaggatttcgcaaactaatttagggtcaagt
gatttaggaatatcaggagtcaatagcggagttgcaagtaaaaatatcaactttagtaatccttcagggatgttgtacgtcactataagtgatgtt
tattcaggatctccgacattgaccattgaataattttaaacgactaatttttagtcgtttttttattttggataaaaggagcaaacaaatggatattaa
ctggaaattgagattcaaaaacaaagcagtactaactggtttagttggagcattgttgctatttatcaagcaagtcacggatttattcggattaga
tttatctactcaattaaatcaagctagcgcaattataggcgctatcctcacgttacttacaggtattggcgttattactgacccaacgtcaaaagg
cgtctcagattcatctatagcacagacatatcaagcgcctagagatagcaaaaaagaagaacaacaagttacgtggaaatcatcacaagac
agtagtttaacgccggaattaagcgcgaaagcaccaaaagaatatgatacatcacaacctttcacagacgcctctaacgatgttggctttgat
gtgaatgagtatcatcatggaggtggcgacaatgcaagcaaaattaactaaaaatgagtttatagagtggttgaaaacttctgagggaaaaca
attcaatgtggacttatggtatggatttcaatgctttgattatgccaatgctggttggaaagttttgtttggattacttctaaaaggtttaggtgcaaa
agatattccgttcgctaacaacttcgacggattagctactgtataccaaaatacaccggacttcttagcacaacctggcgacatggtggtattc
ggtagcaactacggtgctggatatggtcacgttgcatgggtaattgaagcaactttagattacatcattgtatatgagcagaattggctaggcg
gtggctggactgacggaatcgaacaacccggctggggttgggaaaaagttacaagacgacaacatgcttatgatttccctatgtggtttatcc
gtccgaattttaaaagtgagacagcgccacgatcagttcaatctcctacacaagcacctaaaaaagaaacagctaagccacaacctaaagc
agtagaacttaaaatcatcaaagatgtggttaaaggttatgacctacctaagcgtggtagtaaccctaaaggtatagttatacacaacgacgca
gggagcaaaggggcgactgctgaagcatatcgtaacggattagtaaatgcacctttatcaagattagaagcgggcattgcgcatagttacgt
atcaggcaacacagtttggcaagccttagatgaatcacaagtaggttggcataccgctaatcaaataggtaataaatattattacggtattgaa
gtatgtcaatcaatgggcgcagataacgcgacattcttaaaaaatgaacaggcaactttccaagaatgcgctagattgttgaaaaaatgggg
attaccagcaaacagaaatacaatcagattgcacaatgaatttacttcaacatcatgccctcatagaagttcggttttacacactggttttgaccc
agtaactcgcggtctattgccagaagacaagcggttgcaacttaaagactactttatcaagcagattagggcgtacatggatggtaaaatacc
ggttgccactgtctctaatgagtcaagcgcttcaagtaatacagttaaaccagttgcaagtgcatggaaacgtaataaatatggtacttactaca
tggaagaaagtgctagattcacaaacggcaatcaaccaatcacagtaagaaaagtggggccattcttatcttgtccagtgggttatcagttcc
aacctggtgggtattgtgattatacagaagtgatgttacaagatggtcatgtttgggtaggatatacatgggaggggcaacgttattacttgcct
attagaacatggaatggttctgccccacctaatcagatattaggtgacttatggggagaaatcagttagaatgacatagtcatgtctatttaagc
aggtgcgttacatacctgctttctatttacatttaaagataaaatgtgctattattttactagaactttttaacatttctctcaagatttaaatgtagat
aacaggcaggtactacggtacttgcctatttttttatgcaaattttaaaaaacactttactaataaacatttgtttagtataattatatttgtaggttag
ttgatgacttacaaattatgtgtaaggaggtgaaaagcctcatgctagacataataaaaacacttctagaacatcaagtattggcagtactgataatt
ccagaagtgttaaaacaacttagagaatggcatctcggctacctagaccgaaagccaaacaacaaagattaacattatgcttggagcctgat
ggctcctccttacacttatataatataatattatttggaggttttcaattatgacagaacaaatgtatttaatattgtttttattaagcctaccattgtt
attatttatcgggagaaagacacatttttattgtttagataaaaagaatggacgtagataatatgagtgattataaattaaaaataattgaattgatcaa
aagtgatataacaggttaccaaattcacaaacaaactggcgtagcgcaatatgtaatttcacaattaaggcaaggaaagcgcgaagtagata
acttaactttaaatacaactgaaaaactatacagttacgcacgacaagtgttataatataaatgtgaaatggtcattcttgaaatgactcggtcgc
tactggcacagaccgtttaaagtgtcaccacaacatgaactgagaattcatatgacgttgctgacgagcgacaaagctctgtgttcctgaatg
ggagtaggtttgtgtggtggtataatttagtaacagcatagactgtctatagcaaagttgccgaagagattctaaacgtatttataaatacgtgg
cccttgctagataaccgcatcttaactgatgcggttatttttatccccacacaaccaacaaaaccacaccacctattaatttaggagtgtggttgt
tttaatatgtgaagctaaaataactacaaatgataccatttttgataccattttgttgtaaaacagaaaaaataaggaaaataaaaaaggcaaaaa
aacgcattaaatcaacgtttattgtctcatgaaatttaaatgtatataaatttca

A List of Phage that Work with SaPIs

Different SaPIs are linked to different helper phages (see FIG. 3 below)

One can mutates the helper phage to only contain structural genes to direct the phage to package in smaller capsids. If only looking at the genes responsible for small capsid packaging (cpmA and cpmB) these are highly conserved among staphylococci indicating that they will function to redirect packaging in a variety of phages broader than the list below (FIG. 3).

TABLE 1
Example Bacteria
Optionally, the host cells are selected from this Table and/or the target cells are selected from this Table (eg, wherein the host and target cells are of a different species; or of the same species but are
different strain or the host cells are engineered but the target cells are wild-type or vice versa). For example the host cells are E coli cells and the target cells are
C dificile, E coli, Akkermansia, Enterobacteriacea, Ruminococcus, Faecalibacterium, Firmicutes, Bacteroides, Salmonella, Klebsiella, Pseudomonas, Acinterobacter or Streptococcus cells.

| | | | | |
|---|---|---|---|---|
| Abiotrophia | Acidocella | Actinomyces | Alkalilimnicola | Aquaspirillum |
| Abiotrophia defectiva | Acidocella aminolytica | Actinomyces bovis | Alkalilimnicola ehrlichii | Aquaspirillum polymorphum |
| Acaricomes | Acidocella facilis | Actinomyces denticolens | Alkaliphilus | Aquaspirillum |
| Acaricomes phytoseiuli | Acidomonas | Actinomyces europaeus | Alkaliphilus oremlandii | putridiconchylium |
| Acetitomaculum | Acidomonas methanolica | Actinomyces georgiae | Alkaliphilus transvaalensis | Aquaspirillum serpens |
| Acetitomaculum ruminis | Acidothermus | Actinomyces gerencseriae | Allochromatium | Aquimarina |
| Acetivibrio | Acidothermus cellulolyticus | Actinomyces | Allochromatium vinosum | Aquimarina latercula |
| Acetivibrio cellulolyticus | Acidovorax | hordeovulneris | Alloiococcus | Arcanobacterium |
| Acetivibrio ethanolgignens | Acidovorax anthurii | Actinomyces howellii | Alloiococcus otitis | Arcanobacterium |
| Acetivibrio multivorans | Acidovorax caeni | Actinomyces hyovaginalis | Allokutzneria | haemolyticum |
| Acetoanaerobium | Acidovorax cattleyae | Actinomyces israelii | Allokutzneria albata | Arcanobacterium pyogenes |
| Acetoanaerobium noterae | Acidovorax citrulli | Actinomyces johnsonii | Altererythrobacter | Archangium |
| Acetobacter | Acidovorax defluvii | Actinomyces meyeri | Altererythrobacter ishigakiensis | Archangium gephyra |
| Acetobacter aceti | Acidovorax delafieldii | Actinomyces naeslundii | Altermonas | Arcobacter |
| Acetobacter cerevisiae | Acidovorax facilis | Actinomyces neuii | Altermonas haloplanktis | Arcobacter butzleri |
| Acetobacter cibinongensis | Acidovorax konjaci | Actinomyces odontolyticus | Altermonas macleodii | Arcobacter cryaerophilus |
| Acetobacter estunensis | Acidovorax temperans | Actinomyces oris | Alysiella | Arcobacter halophilus |
| Acetobacter fabarum | Acidovorax valerianellae | Actinomyces radingae | Alysiella crassa | Arcobacter nitrofigilis |
| Acetobacter ghanensis | Acinetobacter | Actinomyces slackii | Alysiella filiformis | Arcobacter skirrowii |
| Acetobacter indonesiensis | Acinetobacter baumannii | Actinomyces turicensis | Aminobacter | Arhodomonas |
| Acetobacter lovaniensis | Acinetobacter baylyi | Actinomyces viscosus | Aminobacter aganoensis | Arhodomonas aquaeolei |
| Acetobacter malorum | Acinetobacter bouvetii | Actinoplanes | Aminobacter aminovorans | Arsenophonus |
| Acetobacter nitrogenifigens | Acinetobacter calcoaceticus | Actinoplanes auranticolor | Aminobacter niigataensis | Arsenophonus |
| Acetobacter oeni | Acinetobacter gerneri | Actinoplanes brasiliensis | Aminobacterium | nasoniae |
| Acetobacter orientalis | Acinetobacter haemolyticus | Actinoplanes consettensis | Aminobacterium mobile | Arthrobacter |
| Acetobacter orleanensis | Acinetobacter johnsonii | Actinoplanes deccanensis | Aminomonas | Arthrobacter agilis |
| Acetobacter pasteurianus | Acinetobacter junii | Actinoplanes derwentensis | Aminomonas paucivorans | Arthrobacter albus |
| Acetobacter pornorurn | Acinetobacter lwoffi | Actinoplanes digitatis | Ammoniphilus | Arthrobacter aurescens |
| Acetobacter senegalensis | Acinetobacter parvus | Actinoplanes durhamensis | Ammoniphilus oxalaticus | Arthrobacter chlorophenolicus |
| Acetobacter xylinus | Acinetobacter radioresistens | Actinoplanes ferrugineus | Ammoniphilus oxalivorans | Arthrobacter citreus |
| Acetobacterium | Acinetobacter schindleri | Actinoplanes globisporus | Amphibacillus | Arthrobacter crystallopoietes |
| Acetobacterium bakii | Acinetobacter soli | Actinoplanes humidus | Amphibacillus xylanus | Arthrobacter cumminsii |
| Acetobacterium carbinolicum | Acinetobacter tandoii | Actinoplanes italicus | Amphritea | Arthrobacter globiformis |
| Acetobacterium dehalogenans | Acinetobacter tjernbergiae | Actinoplanes liguriensis | Amphritea balenae | Arthrobacter |
| Acetobacterium fimetarium | Acinetobacter towneri | Actinoplanes lobatus | Amphritea japonica | histidinolovorans |
| Acetobacterium malicum | Acinetobacter ursingii | Actinoplanes missouriensis | Amycolatopsis | Arthrobacter ilicis |
| Acetobacterium paludosum | Acinetobacter venetianus | Actinoplanes palleronii | Amycolatopsis alba | Arthrobacter luteus |
| Acetobacterium tundrae | Acrocarpospora | Actinoplanes philippinensis | Amycolatopsis albidoflavus | Arthrobacter methylotrophus |
| Acetobacterium wieringae | Acrocarpospora corrugata | Actinoplanes rectilineatus | Amycolatopsis azurea | Arthrobacter mysorens |
| Acetobacterium woodii | Acrocarpospora | Actinoplanes regularis | Amycolatopsis coloradensis | Arthrobacter nicotianae |
| Acetofilamentum | macrocephala | Actinoplanes | Amycolatopsis lurida | Arthrobacter nicotinovorans |
| Acetofilamentum rigidum | Acrocarpospora pleiomorpha | teichomyceticus | Amycolatopsis mediterranei | Arthrobacter oxydans |
| Acetohalobium | Actibacter | Actinoplanes utahensis | Amycolatopsis rifamycinica | Arthrobacter pascens |
| Acetohalobium arabaticum | Actibacter sediminis | Actinopolyspora | Amycolatopsis rubida | Arthrobacter |
| Acetomicrobium | Actinoalloteichus | Actinopolyspora halophila | Amycolatopsis sulphurea | phenanthrenivorans |
| Acetomicrobium faecale | Actinoalloteichus | Actinopolyspora mortivallis | Amycolatopsis tolypomycina | Arthrobacter |
| Acetomicrobium flavidum | cyanogriseus | Actinosynnema | Anabaena | polychromogenes |
| Acetonema | Actinoalloteichus | Actinosynnema mirum | Anabaena cylindrica | Atrhrobacter protophormiae |
| Acetonema longum | hymeniacidonis | Actinotalea | Anabaena flos-aquae | Arthrobacter |
| Acetothermus | Actinoalloteichus spitiensis | Actinotalea fermentans | Anabaena variabilis | psychrolactophilus |
| Acetothermus paucivorans | Actinobaccillus | Aerococcus | Anaeroarcus | Arthrobacter ramosus |
| Acholeplasma | Actinobacillus capsulatus | Aerococcus sanguinicola | Anaeroarcus burkinensis | Arthrobacter sulfonivorans |
| Acholeplasma axanthum | Actinobacillus delphinicola | Aerococcus urinae | Anaerobaculum | Arthrobacter sulfureus |
| Acholeplasma brassicae | Actinobacillus hominis | Aerococcus urinaeequi | Anaerobaculum mobile | Arthrobacter uratoxydans |
| Acholeplasma cavigenitalium | Actinobacillus indolicus | Aerococcus urinaehominis | Anaerobiospirillum | Arthrobacter ureafaciens |
| Acholeplasma equifetale | Actinobacillus lignieresii | Aerococcus viridans | Anaerobiospirillum | Arthrobacter viscosus |
| Acholeplasma granularum | Actinobacillus minor | Aeromicrobium | succiniciproducens | Arthrobacter woluwensis |
| Acholeplasma hippikon | Actinobacillus muris | Aeromicrobium erythreum | Anaerobiospirillum thomasii | Asaia |
| Acholeplasma laidlawii | Actinobacillus | Aeromonas | Anaerococcus | Asaia bogorensis |
| Acholeplasma modicum | pleuropneumoniae | Aeromonas | Anaerococcus hydrogenalis | Asanoa |
| Acholeplasma morum | Actinobacillus porcinus | allosaccharophila | Anaerococcus lactolyticus | Asanoa ferruginea |
| Acholeplasma multilocale | Actinobacillus rossii | Aeromonas bestiarum | Anaerococcus prevotii | Asticcacaulis |
| Acholeplasma oculi | Actinobacillus scotiae | Aeromonas caviae | Anaerococcus tetradius | Asticcacaulis biprosthecium |
| Acholeplasma palmae | Actinobacillus seminis | Aeromonas encheleia | Anaerococcus vaginalis | Asticcacaulis excentricus |
| Acholeplasma parvum | Actinobacillus succinogenes | Aeromonas | Anaerofustis | Atopobacter |
| Acholeplasma pleciae | Actinobaccillus suis | enteropelogenes | Anaerofustis stercorihominis | Atopobacter phocae |
| Acholeplasma vituli | Actinobacillus ureae | Aeromonas eucrenophila | Anaeromusa | Atopobium |
| Achromobacter | Actinobaculum | Aeromonas ichthiosmia | Anaeromusa acidaminophila | Atopobium fossor |
| Achromobacter denitrificans | Actinobaculum massiliense | Aeromonas jandaei | Anaeromyxobacter | Atopobium minutum |
| Achromobacter insolitus | Actinobaculum schaalii | Aeromonas media | Anaeromyxobacter | Atopobium parvulum |
| Achromobacter piechaudii | Actinobaculum suis | Aeromonas popoffii | dehalogenans | Atopobium rimae |
| Achromobacter ruhlandii | Actinomyces urinale | Aeromonas sobria | Anaerorhabdus | Atopobium vaginae |
| Achromobacter spanius | Actinocatenispora | Aeromonas veronii | Anaerorhabdus furcosa | Aureobacterium |
| Acidaminobacter | Actinocatenispora rupis | Agrobacterium | Anaerosinus | Aureobacterium barkeri |
| Acidaminobacter | Actinocatenispora | Agrobacterium | Anaerosinus glycerini | Aurobacterium |
| hydrogenoformans | thailandica | gelatinovorum | Anaerovirgula | Aurobacterium liquefaciens |
| Acidaminococcus | Actinocatenispora sera | Agrococcus | Anaerovirgula multivorans | Avibacterium |
| Acidaminococcus fermentans | Actinocorallia | Agrococcus citreus | Ancalomicrobium | Avibacterium avium |
| Acidaminococcus intestini | Actinocorallia aurantiaca | Agrococcus jenensis | Ancalomicrobium adetum | Avibacterium gallinarum |
| Acidicaldus | Actinocorallia aurea | Agromonas | Ancylobacter | Avibacterium paragallinarum |
| Acidicaldus organivorans | Actinocorallia cavernae | Agromonas oligotrophica | Ancylobacter aquaticus | Avibacterium volantium |
| Acidimicrobium | Actinocorallia glomerata | Agromyces | Aneurinibacillus | Azoarcus |
| Acidimicrobium ferrooxidans | Actinocorallia herbida | Agromyces fucosus | Aneurinibacillus aneurinilyticus | Azoarcus indigens |
| Acidiphilium | Actinocorallia libanotica | Agromyces hippuratus | Aneurinibacillus migulanus | Azoarcus tolulyticus |
| Acidiphilium acidophilum | Actinocorallia longicatena | Agromyces luteolus | Aneurinibacillus | Azoarcus toluvorans |
| Acidiphilium angustum | Actinomadura | Agromyces mediolanus | thermoaerophilus | Azohydromonas |
| Acidiphilium cryptum | Actinomadura alba | Agromyces ramosus | Angiococcus | Azohydromonas australica |
| Acidiphilium multivorum | Actinomadura atramentaria | Agromyces rhizospherae | Angiococcus disciformis | Azohydromonas lata |
| Acidiphilium organovorum | Actinomadura | Akkermansia | Angulomicrobium | Azomonas |
| Acidiphilium rubrum | bangladeshensis | Akkermansia muciniphila | Angulomicrobium tetraedrale | Azomonas agilis |
| Acidisoma | Actinomadura catellatispora | Albidiferax | Anoxybacillus | Azomonas insignis |
| Acidisoma sibiricum | Actinomadura chibensis | Albidiferax ferrireducens | Anoxybacillus pushchinoensis | Azomonas macrocytogenes |
| Acidisoma tundrae | Actinomadura chokoriensis | Albidovulum | Aquabacterium | Azorhizobium |
| Acidisphaera | Actinomadura citrea | Albidovulum inexpectatum | Aquabacterium commune | Azorhizobium caulinodans |
| Acidisphaera rubrifaciens | Actinomadura coerulea | Alcaligenes | Aquabacterium parvum | Azorhizophilus |
| Acidithiobacillus | Actinomadura echinospora | Alcaligenes denitrificans | | Azorhizophilus paspali |
| Acidithiobacillus albertensis | Actinomadura fibrosa | Alcaligenes faecalis | | Azospirillum |
| Acidithiobacillus caldus | Actinomadura formosensis | Alcanivorax | | Azospirillum brasilense |
| Acidithiobacillus ferrooxidans | Actinomadura hibisca | Alcanivorax borkumensis | | Azospirillum halopraeferens |
| Acidithiobacillus thiooxidans | Actinomadura kijaniata | Alcanivorax jadensis | | Azospirillum irakense |
| Acidobacterium | Actinomadura latina | Algicola | | Azotobacter |
| Acidobacterium capsulatum | Actinomadura livida | Algicola bacteriolytica | | Azotobacter beijerinckii |
| | Actinomadura | Alicyclobacillus | | Azotobacter chroococcum |
| | luteofluorescens | Alicyclobacillus | | Azotobacter nigricans |
| | Actinomadura macra | disulfidooxidans | | Azotobacter salinestris |
| | Actinomadura madurae | Alicyclobacillus | | Azotobacter vinelandii |
| | Actinomadura oligospora | sendaiensis | | |
| | Actinomadura pelletieri | Alicyclobacillus vulcanalis | | |
| | Actinomadura rubrobrunea | Alishewanella | | |
| | Actinomadura rugatobispora | Alishewanella fetalis | | |
| | Actinomadura umbrina | Alkalibacillus | | |
| | Actinomadura | Alkalibacillus | | |
| | verrucosospora | haloalkaliphilus | | |
| | Actinomadura vinacea | | | |
| | Actinomadura viridilutea | | | |
| | Actinomadura viridis | | | |
| | Actinomadura yumaensis | | | |
| Bacillus | Bacteroides | Bibersteinia | Borrelia | Brevinema |
| [see below] | Bacteroides caccae | Bibersteinia trehalosi | Borrelia afzelii | Brevinema andersonii |
| Bacteriovorax | Bacteroides coagulans | Bifidobacterium | Borrelia americana | Brevundimonas |
| Bacteriovorax stolpii | Bacteroides eggerthii | Bifidobacterium adolescentis | Borrelia burgdorferi | Brevundimonas alba |
| | Bacteroides fragilis | Bifidobacterium angulatum | Borrelia carolinensis | Brevundimonas aurantiaca |
| | Bacteroides galacturonicus | Bifidobacterium animalis | Borrelia coriaceae | Brevundimonas diminuta |
| | Bacteroides helcogenes | Bifidobacterium asteroides | Borrelia garinii | Brevundimonas intermedia |
| | Bacteroides ovatus | Bifidobacterium bifidum | Borrelia japonica | Brevundimonas subvibrioides |
| | Bacteroides pectinophilus | Bifidobacterium boum | Bosea | Brevundimonas vancanneytii |
| | Bacteroides pyogenes | Bifidobacterium breve | Bosea minatitlanensis | Brevundimonas variabilis |
| | Bacteroides salyersiae | Bifidobacterium catenulatum | Bosea thiooxidans | Brevundimonas vesicularis |
| | Bacteroides stercoris | Bifidobacterium choerinum | Brachybacterium | Brochothrix |
| | Bacteroides suis | Bifidobacterium coryneforme | Brachybacterium | Brochothrix campestris |
| | Bacteroides tectus | Bifidobacterium cuniculi | alimentarium | Brochothrix thermosphacta |
| | Bacteroides thetaiotaomicron | Bifidobacterium dentium | Brachybacterium faecium | Brucella |
| | Bacteroides uniformis | Bifidobacterium gallicum | Brachybacterium | Brucella canis |
| | Bacteroides ureolyticus | Bifidobacterium gallinarum | paraconglomeratum | Brucella neotomae |
| | Bacteroides vulgatus | Bifidobacterium indicum | Brachybacterium rhamnosum | Bryobacter |
| | Balnearium | Bifidobacterium longum | Brachybacterium | Bryobacter aggregatus |
| | Balnearium lithotrophicum | Bifidobacterium | tyrofermentans | Burkholderia |
| | Balneatrix | magnumBifidobacterium | Brachyspira | Burkholderia ambifaria |
| | Balneatrix alpica | merycicum | Brachyspira alvinipulli | Burkholderia andropogonis |
| | Balneola | Bifidobacterium minimum | Brachyspira hyodysenteriae | Burkholderia anthina |
| | Balneola vulgaris | Bifidobacterium | Brachyspira innocens | Burkholderia caledonica |
| | Barnesiella | pseudocatenulatum | Brachyspira murdochii | Burkholderia caryophylli |
| | Barnesiella viscericola | Bifidobacterium | Brachyspira | Burkholderia cenocepacia |
| | Bartonella | pseudolongum | pilosicoli | Burkholderia cepacia |
| | Bartonella alsatica | Bifidobacterium pullorum | Bradyrhizobium | Burkholderia cocovenenans |
| | Bartonella bacilliformis | Bifidobacterium ruminantium | Bradyrhizobium canariense | Burkholderia dolosa |
| | Bartonella clarridgeiae | Bifidobacterium saeculare | Bradyrhizobium elkanii | Burkholderia fungorum |
| | Bartonella doshiae | Bifidobacterium subtile | Bradyrhizobium japonicum | Burkholderia glathei |
| | Bartonella elizabethae | Bifidobacterium | Bradyrhizobium liaoningense | Burkholderia glumae |
| | Bartonella grahamii | thermophilum | Brenneria | Burkholderia graminis |
| | Bartonella henselae | Bilophila | Brenneria alni | Burkholderia kururiensis |
| | Bartonella rochalimae | Bilophila wadsworthia | Brenneria nigrifluens | Burkholderia multivorans |
| | Bartonella vinsonii | Biostraticola | Brenneria quercina | Burkholderia phenazinium |
| | Bavariicoccus | Biostraticola tofi | Brenneria quercina | Burkholderia plantarii |
| | Bavariicoccus seileri | Bizionia | Brenneria salicis | Burkholderia pyrrocinia |
| | Bdellovibrio | Bizionia argentinensis | Brevibacillus | Burkholderia silvatlantica |
| | Bdellovibrio bacteriovorus | Blastobacter | Brevibacillus agri | Burkholderia stabilis |
| | Bdellovibrio exovorus | Blastobacter capsulatus | Brevibacillus borstelensis | Burkholderia thailandensis |
| | Beggiatoa | Blastobacter denitrificans | Brevibacillus brevis | Burkholderia tropica |
| | Beggiatoa alba | Blastococcus | Brevibacillus centrosporus | Burkholderia unamae |
| | Beijerinckia | Blastococcus aggregatus | Brevibacillus choshinensis | Burkholderia vietnamiensis |
| | Beijerinckia derxii | Blastococcus saxobsidens | Brevibacillus invocatus | Buttiauxella |
| | Beijerinckia fluminensis | Blastochloris | Brevibacillus laterosporus | Buttiauxella agrestis |
| | Beijerinckia indica | Blastochloris viridis | Brevibacillus parabrevis | Buttiauxella brennerae |
| | Beijerinckia mobilis | Blastomonas | Brevibacillus reuszeri | Buttiauxella ferragutiae |
| | Belliella | Blastomonas natatoria | Brevibacterium | Buttiauxella gaviniae |
| | Belliella baltica | Blastopirellula | Brevibacterium abidum | Buttiauxella izardii |
| | Bellilinea | Blastopirellula marina | Brevibacterium album | Buttiauxella noackiae |
| | Bellilinea caldifistulae | Blautia | Brevibacterium aurantiacum | Buttiauxella warmboldiae |
| | Belnapia | Blautia coccoides | Brevibacterium celere | Butyrivibrio |
| | Belnapia moabensis | Blautia hansenii | Brevibacterium epidermidis | Butyrivibrio fibrisolvens |
| | Bergeriella | Blautia producta | Brevibacterium | Butyrivibrio hungatei |
| | Bergeriella denitrificans | Blautia wexlerae | frigoritolerans | Butyrivibrio proteoclasticus |
| | Beutenbergia | Bogoriella | Brevibacterium halotolerans | |
| | Beutenbergia cavernae | Bogoriella caseilytica | Brevibacterium iodinum | |
| | | Bordetella | Brevibacterium linens | |
| | | Bordetella avium | Brevibacterium lyticum | |
| | | Bordetella bronchiseptica | Brevibacterium mcbrellneri | |
| | | Bordetella hinzii | Brevibacterium otitidis | |
| | | Bordetella holmesii | Brevibacterium oxydans | |
| | | Bordetella parapertussis | Brevibacterium paucivorans | |
| | | Bordetella pertussis | Brevibacterium stationis | |
| | | Bordetella petrii | | |
| | | Bordetella trematum | | |
| Bacillus | | | | |
| B. acidiceler | B. aminovorans | B. glucanolyticus | B. taeanensis | B. lautus |
| B. acidicola | B. amylolyticus | B. gordonae | B. tequilensis | B. lehensis |
| B. acidiproducens | B. andreesenii | B. gottheilii | B. thermantarcticus | B. lentimorbus |
| B. acidocaldarius | B. aneurinilyticus | B. graminis | B. thermoaerophilus | B. lentus |
| B. acidoterrestris | B. anthracis | B. halmapalus | B. thermoamylovorans | B. licheniformis |
| B. aeolius | B. aquimaris | B. haloalkaliphilus | B. thermocatenulatus | B. ligniniphilus |
| B. aerius | B. arenosi | B. halochares | B. thermocloacae | B. litoralis |
| B. aerophilus | B. arseniciselenatis | B. halodenitrificans | B. thermocopriae | B. locisalis |
| B. agaradhaerens | B. arsenicus | B. halodurans | B. thermodenitrificans | B. luciferensis |
| B. agri | B. aurantiacus | B. halophilus | B. thermoglucosidasius | B. luteolus |
| B. aidingensis | B. arvi | B. halosaccharovorans | B. thermolactis | B. luteus |
| B. akibai | B. aryabhattai | B. hemicellulosilyticus | B. thermoleovorans | B. macauensis |
| B. alcalophilus | B. asahii | B. hemicentroti | B. thermophilus | B. macerans |
| B. algicola | B. atrophaeus | B. herbersteinensis | B. thermoruber | B. macquariensis |
| B. alginolyticus | B. axarquiensis | B. horikoshii | B. thermosphaericus | B. macyae |
| B. alkalidiazotrophicus | B. azotofixans | B. horneckiae | B. thiaminolyticus | B. malacitensis |
| B. alkalinitrilicus | B. azotoformans | B. horti | B. thioparans | B. mannanilyticus |
| B. alkalisediminis | B. badius | B. huizhouensis | B. thuringiensis | B. marisflavi |
| B. alkalitelluris | B. barbaricus | B. humi | B. tianshenii | B. marismortui |
| B. altitudinis | B. bataviensis | B. hwajinpoensis | B. trypoxylicola | B. marmarensis |
| B. alveayuensis | B. beijingensis | B. idriensis | B. tusciae | B. massiliensis |
| B. alvei | B. benzoevorans | B. indicus | B. validus | B. megaterium |
| B. amyloliquefaciens | B. beringensis | B. infantis | B. vallismortis | B. mesonae |
| B. | B. berkeleyi | B. infernus | B. vedderi | B. methanolicus |
| a. subsp. amyloliquefaciens | B. beveridgei | B. insolitus | B. velezensis | B. methylotrophicus |
| B. a. subsp. plantarum | B. bogoriensis | B. invictae | B. vietnamensis | B. migulanus |
| | B. boroniphilus | B. iranensis | B. vireti | B. mojavensis |
| B. dipsosauri | B. borstelensis | B. isabeliae | B. vulcani | B. mucilaginosus |
| B. drentensis | B. brevis Migula | B. isronensis | B. wakoensis | B. muralis |
| B. edaphicus | B. butanolivorans | B. jeotgali | B. weihenstephanensis | B. murimartini |
| B. ehimensis | B. canaveralius | B. kaustophilus | B. xiamenensis | B. mycoides |
| B. eiseniae | B. carboniphilus | B. kobensis | B. xiaoxiensis | B. naganoensis |
| B. enclensis | B. cecembensis | B. kochii | B. zhanjiangensis | B. nanhaiensis |
| B. endophyticus | B. cellulosilyticus | B. kokeshiiformis | B. peoriae | B. nanhaiisediminis |
| B. endoradicis | B. centrosporus | B. koreensis | B. persepolensis | B. nealsonii |
| B. farraginis | B. cereus | B. korlensis | B. persicus | B. neidei |
| B. fastidiosus | B. chagannorensis | B. kribbensis | B. pervagus | B. neizhouensis |
| B. fengqiuensis | B. chitinolyticus | B. krulwichiae | B. plakortidis | B. niabensis |
| B. firmus | B. chondroitinus | B. laevolacticus | B. pocheonensis | B. niacini |
| B. flexus | B. choshinensis | B. larvae | B. polygoni | B. novalis |
| B. foraminis | B. chungangensis | B. laterosporus | B. polymyxa | B. oceanisediminis |
| B. fordii | B. cibi | B. salexigens | B. popilliae | B. odysseyi |
| B. formosus | B. circulans | B. saliphilus | B. pseudalcalophilus | B. okhensis |
| B. fortis | B. clarkii | B. schlegelii | B. pseudofirmus | B. okuhidensis |
| B. fumarioli | B. clausii | B. sediminis | B. pseudomycoides | B. oleronius |
| B. funiculus | B. coagulans | B. selenatarsenatis | B. psychrodurans | B. oryzaecorticis |
| B. fusiformis | B. coahuilensis | B. selenitireducens | B. psychrophilus | B. oshimensis |
| B. galactophilus | B. cohnii | B. seohaeanensis | B. psychrosaccharolyticus | B. pabuli |
| B. galactosidilyticus | B. composti | B. shacheensis | B. psychrotolerans | B. pakistanensis |
| B. galliciensis | B. curdlanolyticus | B. shackletonii | B. pulvifaciens | B. pallidus |
| B. gelatini | B. cycloheptanicus | B. siamensis | B. pumilus | B. pallidus |
| B. gibsonii | B. cytotoxicus | B. silvestris | B. purgationiresistens | B. panacisoli |
| B. ginsengi | B. daliensis | B. simplex | B. pycnus | B. panaciterrae |
| B. ginsengihumi | B. decisifrondis | B. siralis | B. qingdaonensis | B. pantothenticus |
| B. ginsengisoli | B. decolorationis | B. smithii | B. qingshengii | B. parabrevis |
| B. globisporus (eg, B. | B. deserti | B. soli | B. reuszeri | B. paraflexus |
| g. subsp. Globisporus; or B. | | B. solimangrovi | B. rhizosphaerae | B. pasteurii |
| g. subsp. Marinus) | | B. solisalsi | B. rigui | B. patagoniensis |
| | | B. songklensis | B. ruris | |
| | | B. sonorensis | B. safensis | |
| | | B. sphaericus | B. salarius | |
| | | B. sporothermodurans | | |
| | | B. stearothermophilus | | |
| | | B. stratosphericus | | |
| | | B. subterraneus | | |
| | | B. subtilis (eg, B. | | |
| | | s. subsp. Inaquosorum, or B. | | |
| | | s. subsp. Spizizenr, or B. | | |
| | | s. subsp. Subtilis) | | |
| Caenimonas | Campylobacter | Cardiobacterium | Catenuloplanes | Curtobacterium |
| Caenimonas koreensis | Campylobacter coli | Cardiobacterium hominis | Catenuloplanes atrovinosus | Curtobacterium albidum |
| Caldalkalibacillus | Campylobacter concisus | Carnimonas | Catenuloplanes castaneus | Curtobacterium citreus |
| Caldalkalibacillus uzonensis | Campylobacter curvus | Carnimonas nigrificans | Catenuloplanes crispus | |
| Caldanaerobacter | Campylobacter fetus | Carnobacterium | Catenuloplanes indicus | |
| Caldanaerobacter subterraneus | Campylobacter gracilis | Carnobacterium alterfunditum | Catenuloplanes japonicus | |
| Caldanaerobius | Campylobacter helveticus | Carnobacterium divergens | Catenuloplanes nepalensis | |
| Caldanaerobius fijiensis | Campylobacter hominis | Carnobacterium funditum | Catenuloplanes niger | |
| Caldanaerobius | Campylobacter hyointestinalis | Carnobacterium gallinarum | Chryseobacterium | |
| polysaccharolyticus | Campylobacter jejuni | Carnobacterium | Chryseobacterium | |
| Caldanaerobius zeae | Campylobacter lari | maltaromaticum | balustinum | |
| Caldanaerovirga | Campylobacter mucosalis | Carnobacterium mobile | Citrobacter | |
| Caldanaerovirga acetigignens | Campylobacter rectus | Carnobacterium viridans | C. amalonaticus | |
| Caldicellulosiruptor | Campylobacter showae | Caryophanon | C. braakii | |
| Caldicellulosiruptor bescii | Campylobacter sputorum | Caryophanon latum | C. diversus | |
| Caldicellulosiruptor kristjanssonii | Campylobacter upsaliensis | Caryophanon tenue | C. farmeri | |
| Caldicellulosiruptor owensensis | Capnocytophaga | Catellatospora | C. freundii | |
| | Capnocytophaga canimorsus | Catellatospora citrea | C. gillenii | |
| | Capnocytophaga cynodegmi | Catellatospora | C. koseri | |
| | Capnocytophaga gingivalis | methionotrophica | C. murliniae | |
| | Capnocytophaga granulosa | Catenococcus | C. pasteurii^{[1]} | |
| | Capnocytophaga haemolytica | Catenococcus thiocycli | C. rodentium | |
| | Capnocytophaga ochracea | | C. sedlakii | |
| | Capnocytophaga sputigena | | C. werkmanii | |
| | | | C. youngae | |
| | | | Clostridium | |
| | | | (see below) | |
| | | | Coccochloris | |
| | | | Coccochloris elabens | |
| | | | Corynebacterium | |
| | | | Corynebacterium flavescens | |
| | | | Corynebacterium variabile | |
| Clostridium | | | | |

Clostridium absonum, Clostridium aceticum, Clostridium acetireducens, Clostridium acetobutylicum, Clostridium acidisoli, Clostridium aciditolerans, Clostridium acidurici, Clostridium aerotolerans, Clostridium
aestuarii, Clostridium akagii, Clostridium aldenense, Clostridium aldrichii, Clostridium algidicarni, Clostridium algidixylanolyticum, Clostridium algifaecis, Clostridium algoriphilum, Clostridium alkalicellulosi,
Clostridium aminophilum, Clostridium aminovalericum, Clostridium amygdalinum, Clostridium amylolyticum, Clostridium arbusti, Clostridium arcticum, Clostridium argentinense, Clostridium asparagiforme,
Clostridium aurantibutyricum, Clostridium autoethanogenum, Clostridium baratii, Clostridium barkeri, Clostridium bartlettii, Clostridium beijerinckii, Clostridium bifermentans, Clostridium bolteae, Clostridium
bornimense, Clostridium botulinum, Clostridium bowmanii, Clostridium bryantii, Clostridium butyricum, Clostridium cadaveris, Clostridium caenicola, Clostridium caminithermale, Clostridium carboxidivorans,
Clostridium carnis, Clostridium cavendishii, Clostridium celatum, Clostridium celerecrescens, Clostridium cellobioparum, Clostridium cellulofermentans, Clostridium cellulolyticum, Clostridium cellulosi,
Clostridium cellulovorans, Clostridium chartatabidum, Clostridium chauvoei, Clostridium chromiireducens, Clostridium citroniae, Clostridium clariflavum, Clostridium clostridioforme, Clostridium coccoides,
Clostridium cochlearium, Clostridium colletant, Clostridium colicanis, Clostridium colinum, Clostridium collagenovorans, Clostridium cylindrosporum, Clostridium difficile, Clostridium diolis, Clostridium
disporicum, Clostridium drakei, Clostridium durum, Clostridium estertheticum, Clostridium estertheticum estertheticum, Clostridium estertheticum laramiense, Clostridium fallax, Clostridium felsineum, Clostridium
fervidum, Clostridium fimetarium, Clostridium formicaceticum, Clostridium frigidicarnis, Clostridium frigoris, Clostridium ganghwense, Clostridium gasigenes, Clostridium ghonii, Clostridium glycolicum,
Clostridium glycyrrhizinilyticum, Clostridium grantii, Clostridium haemolyticum, Clostridium halophilum, Clostridium hastiforme, Clostridium hathewayi, Clostridium herbivorans, Clostridium hiranonis,
Clostridium histolyticum, Clostridium homopropionicum, Clostridium huakuii, Clostridium hungatei, Clostridium hydrogeniformans, Clostridium hydroxybenzoicum, Clostridium hylemonae, Clostridium jejuense,
Clostridium indolis, Clostridium innocuum, Clostridium intestinale, Clostridium irregulare, Clostridium isatidis, Clostridium josui, Clostridium kluyveri, Clostridium lactatifermentans, Clostridium lacusfryxellense,
Clostridium laramiense, Clostridium lavalense, Clostridium lentocellum, Clostridium lentoputrescens, Clostridium leptum, Clostridium limosum, Clostridium litorale, Clostridium lituseburense, Clostridium ljungdahlii,
Clostridium lortetii, Clostridium lundense, Clostridium magnum, Clostridium malenominatum, Clostridium mangenotii, Clostridium mayombei, Clostridium methoxybenzovorans, Clostridium methylpentosum,
Clostridium neopropionicum, Clostridium nexile, Clostridium nitrophenolicum, Clostridium novyi, Clostridium oceanicum, Clostridium orbiscindens, Clostridium oroticum, Clostridium oxalicum, Clostridium
papyrosolvens, Clostridium paradoxum, Clostridium paraperfringens (Alias: C. welchii), Clostridium paraputrificum, Clostridium pascui, Clostridium pasteurianum, Clostridium peptidivorans, Clostridium perenne,
Clostridium perfringens, Clostridium pfennigii, Clostridium phytofermentans, Clostridium piliforme, Clostridium polysaccharolyticum, Clostridium populeti, Clostridium propionicum, Clostridium proteoclasticum,
Clostridium proteolyticum, Clostridium psychrophilum, Clostridium puniceum, Clostridium purinilyticum, Clostridium putrefaciens, Clostridium putrificum, Clostridium quercicolum, Clostridium quinii,
Clostridium ramosum, Clostridium rectum, Clostridium roseum, Clostridium saccharobutylicum, Clostridium saccharogumia, Clostridium saccharolyticum, Clostridium saccharoperbutylacetonicum, Clostridium
sardiniense, Clostridium sartagoforme, Clostridium scatologenes, Clostridium schirmacherense, Clostridium scindens, Clostridium septicum, Clostridium sordellii, Clostridium sphenoides, Clostridium spiroforme,
Clostridium sporogenes, Clostridium sporosphaeroides, Clostridium stercorarium, Clostridium stercorarium leptospartum, Clostridium stercorarium stercorarium, Clostridium stercorarium thermolacticum,
Clostridium sticklandii, Clostridium straminisolvens, Clostridium subterminale, Clostridium sufflavum, Clostridium sulfidigenes, Clostridium symbiosum, Clostridium tagluense, Clostridium
tepidiprofundi, Clostridium termitidis, Clostridium tertium, Clostridium tetani, Clostridium tetanomorphum, Clostridium thermaceticum, Clostridium thermautotrophicum, Clostridium thermoalcaliphilum,
Clostridium thermobutyricum, Clostridium thermocellum, Clostridium thermocopriae, Clostridium thermohydrosulfuricum, Clostridium thermolacticum, Clostridium thermopalmarium,
Clostridium thermopapyrolyticum, Clostridium thermosaccharolyticum, Clostridium thermosuccinogenes, Clostridium thermosulfurigenes, Clostridium thiosulfatireducens, Clostridium tyrobutyricum,
Clostridium uliginosum, Clostridium ultunense, Clostridium villosum, Clostridium vincentii, Clostridium viride, Clostridium xylanolyticum, Clostridium xylanovorans

| | | | | |
|---|---|---|---|---|
| Dactylosporangium | Deinococcus | Delftia | Echinicola | |
| Dactylosporangium aurantiacum | Deinococcus aerius | Delftia acidovorans | Echinicola pacifica | |
| Dactylosporangium fulvum | Deinococcus apachensis | Desulfovibrio | Echinicola vietnamensis | |
| Dactylosporangium matsuzakiense | Deinococcus aquaticus | Desulfovibrio desulfuricans | | |
| Dactylosporangium roseum | Deinococcus aquatilis | Diplococcus | | |
| Dactylosporangium thailandense | Deinococcus caeni | Diplococcus pneumoniae | | |
| Dactylosporangium vinaceum | Deinococcus radiodurans | | | |
| | Deinococcus radiophilus | | | |
| Enterobacter | Enterobacter kobei | Faecalibacterium | Flavobacterium | |
| E. aerogenes | E. ludwigii | Faecalibacterium prausnitzii | Flavobacterium antarcticum | |
| E. amnigemis | E. mori | Fangia | Flavobacterium aquatile | |
| E. agglomerans | E. nimipressuralis | Fangia hongkongensis | Flavobacterium aquidurense | |
| E. arachidis | E. oryzae | Fastidiosipila | Flavobacterium balustinum | |
| E. asburiae | E. pulveris | Fastidiosipila sanguinis | Flavobacterium croceum | |
| E. cancerogenous | E. pyrinus | Fusobacterium | Flavobacterium cucumis | |
| E. cloacae | E. radicincitans | Fusobacterium nucleatum | Flavobacterium daejeonense | |
| E. cowanii | E. taylorae | | Flavobacterium defluvii | |
| E. dissolvens | E. turicensis | | Flavobacterium degerlachei | |
| E. gergoviae | E. sakazakii Enterobacter soli | | Flavobacterium | |
| E. helveticus | Enterococcus | | denitrificans | |
| E. hormaechei | Enterococcus durans | | Flavobacterium filum | |
| E. intermedins | Enterococcus faecalis | | Flavobacterium flevense | |
| | Enterococcus faecium | | Flavobacterium frigidarium | |
| | Erwinia | | Flavobacterium mizutaii | |
| | Erwinia hapontici | | Flavobacterium | |
| | Escherichia | | okeanokoites | |
| | Escherichia coli | | | |
| Gaetbulibacter | Haemophilus | Ideonella | Janibacter | |
| Gaetbulibacter saemankumensis | Haemophilus aegyptius | Ideonella azotifigens | Janibacter anophelis | |
| Gallibacterium | Haemophilus aphrophilus | Idiomarina | Janibacter corallicola | |
| Gallibacterium anatis | Haemophilus felis | Idiomarina abyssalis | Janibacter limosus | |
| Gallicola | Haemophilus gallinarum | Idiomarina baltica | Janibacter melonis | |
| Gallicola barnesae | Haemophilus haemolyticus | Idiomarina fontislapidosi | Janibacter terrae | |
| Garciella | Haemophilus influenzae | Idiomarina loihiensis | Jannaschia | |
| Garciella nitratireducens | Haemophilus paracuniculus | Idiomarina ramblicola | Jannaschia cystaugens | |
| Geobacillus | Haemophilus parahaemolyticus | Idiomarina seosinensis | Jannaschia helgolandensis | |
| Geobacillus thermoglucosidasius | Haemophilus parainfluenzae | Idiomarina zobellii | Jannaschia | |
| Geobacillus stearothermophilus | Haemophilus | Ignatzschineria | pohangensis | |
| Geobacter | paraphrohaemolyticus | Ignatzschineria | Jannaschia rubra | |
| Geobacter bemidjiensis | Haemophilus parasuis | larvae | Janthinobacterium | |
| Geobacter bremensis | Haemophilus pittmaniae | Ignavigranum | Janthinobacterium | |
| Geobacter chapellei | Hafnia | Ignavigranum ruoffiae | agaricidamnosum | |
| Geobacter grbiciae | Hafnia alvei | Ilumatobacter | Janthinobacterium lividum | |
| Geobacter hydrogenophilus | Hahella | Ilumatobacter fluminis | Jejuia | |
| Geobacter lovleyi | Hahella ganghwensis | Ilyobacter | Jejuia pallidilutea | |
| Geobacter metallireducens | Halalkalibacillus | Ilyobacter delafieldii | Jeotgalibacillus | |
| Geobacter pelophilus | Halalkalibacillus halophilus | Ilyobacter insuetus | Jeotgalibacillus | |
| Geobacter pickeringii | Helicobacter | Ilyobacter polytropus | alimentarius | |
| Geobacter sulfurreducens | Helicobacter pylori | Ilyobacter tartaricus | Jeotgalicoccus | |
| Geodermatophilus | | | Jeotgalicoccus halotolerans | |
| Geodermatophilus obscurus | | | | |
| Gluconacetobacter | | | | |
| Gluconacetobacter xylinus | | | | |
| Gordonia | | | | |
| Gordonia rubripertincta | | | | |
| Kaistia | Labedella | Listeria ivanovii | Micrococcus | Nesterenkonia |
| Kaistia adipata | Labedella gwakjiensis | L. marthii | Micrococcus luteus | Nesterenkonia holobia |
| Kaistia soli | Labrenzia | L. monocytogenes | Micrococcus lylae | Nocardia |
| Kangiella | Labrenzia aggregata | L. newyorkensis | Moraxella | Nocardia argentinensis |
| Kangiella aquimarina | Labrenzia alba | L. riparia | Moraxella bovis | Nocardia corallina |
| Kangiella | Labrenzia alexandrii | L. rocourtiae | Moraxella nonliquefaciens | Nocardia |
| koreensis | Labrenzia marina | L. seeligeri | Moraxella osloensis | otitidiscaviarum |
| Kerstersia | Labrys | L. weihenstephanensis | Nakamurella | |
| Kerstersia gyiorum | Labrys methylaminiphilus | L. welshimeri | Nakamurella multipartita | |
| Kiloniella | Labrys miyagiensis | Listonella | Nannocystis | |
| Kiloniella laminariae | Labrys monachus | Listonella anguillarum | Nannocystis pusilia | |
| Klebsiella | Labrys okinawensis | Macrococcus | Natranaerobius | |
| K. gramilomatis | Labrys | Macrococcus bovicus | Natranaerobius | |
| K. oxytoca | portucalensis | Marinobacter | thermophilus | |
| K. pneumoniae | Lactobacillus | Marinobacter algicola | Natranaerobius trueperi | |
| K. terrigena | [see below] | Marinobacter bryozoorum | Naxibacter | |
| K. variicola | Laceyella | Marinobacter flavimaris | Naxibacter alkalitolerans | |
| Kluyvera | Laceyella putida | Meiothermus | Neisseria | |
| Kluyvera ascorbata | Lechevalieria | Meiothermus ruber | Neisseria cinerea | |
| Kocuria | Lechevalieria aerocolonigenes | Methylophilus | Neisseria denitrificans | |
| Kocuria roasea | Legionella | Methylophilus methylotrophus | Neisseria gonorrhoeae | |
| Kocuria varians | [see below] | Microbacterium | Neisseria lactamica | |
| Kurthia | Listeria | Microbacterium | Neisseria mucosa | |
| Kurthia zopfii | L. aquatica | ammoniaphilum | Neisseria sicca | |
| | L. booriae | Microbacterium arborescens | Neisseria subflava | |
| | L. cornellensis | Microbacterium liquefaciens | Neptunomonas | |
| | L. fleischmannii | Microbacterium oxydans | Neptunomonas japonica | |
| | L. floridensis | | | |
| | L. grandensis | | | |
| | L. grayi | | | |
| | L. innocua | | | |
| Lactobacillus | | | | |
| L. acetotolerans | L. catenaformis | L. mali | L. parakefiri | L. sakei |
| L. acidifarinae | L. ceti | L. manihotivorans | L. paralimentarius | L. salivarius |
| L. acidipiscis | L. coleohominis | L. mindensis | L. paraplantarum | L. sanfranciscensis |
| L. acidophilus | L. collinoides | L. mucosae | L. pentosus | L. satsumensis |
| Lactobacillus agilis | L. composti | L. murinus | L. perolens | L. secaliphilus |
| L. algidus | L. concavus | L. nagelii | L. plantarum | L. sharpeae |
| L. alimentarius | L. coryniformis | L. namurensis | L. pontis | L. siliginis |
| L. amylolyticus | L. crispatus | L. nantensis | L. protectus | L. spicheri |
| L. amylophilus | L. crustorum | L. oligofermentans | L. psittaci | L. suebicus |
| L. amylotrophicus | L. curvatus | L. oris | L. rennini | L. thailandensis |
| L. amylovorus | L. delbrueckii subsp. bulgaricus | L. panis | L. reuteri | L. ultunensis |
| L. animalis | L. delbrueckii subsp. | L. pantheris | L. rhamnosus | L. vaccinostercus |
| L. antri | delbrueckii | L. parabrevis | L. rimae | L. vaginalis |
| L. apodemi | L. delbrueckii subsp. lactis | L. parabuchneri | L. rogosae | L. versmoldensis |
| L. aviarius | L. dextrinicus | L. paracasei | L. rossiae | L. vini |
| L. bifermentans | L. diolivorans | L. paracollinoides | L. ruminis | L. vitulinus |
| L. brevis | L. equi | L. parafarraginis | L. saerimneri | L. zeae |
| L. buchneri | L. equigenerosi | L. homohiochii | L. jensenii | L. zymae |
| L. camelliae | L. farraginis | L. iners | L. johnsonii | L. gastricus |
| L. casei | L. farciminis | L. ingluviei | L. kalixensis | L. ghanensis |
| L. kitasatonis | L. fermentum | L. intestinalis | L. kefiranofaciens | L. graminis |
| L. kunkeei | L. fornicalis | L. fuchuensis | L. kefiri | L. hammesii |
| L. leichmannii | L. fructivorans | L. gallinarum | L. kimchii | L. hamsteri |
| L. lindneri | L. frumenti | L. gasseri | L. helveticus | L. harbinensis |
| L. malefermentans | | | L. hilgardii | L. hayakitensis |
| Legionella | | | | |
| Legionella adelaidensis | Legionella drancourtii | Candidatus Legionella jeonii | Legionella quinlivanii | |
| Legionella anisa | Legionella dresdenensis | Legionella jordanis | Legionella rowbothamii | |
| Legionella beliardensis | Legionella drozanskii | Legionella lansingensis | Legionella rubrilucens | |
| Legionella birminghamensis | Legionella dumoffii | Legionella londiniensis | Legionella sainthelensi | |
| Legionella bozemanae | Legionella erythra | Legionella longbeachae | Legionella santicrucis | |
| Legionella brunensis | Legionella fairfieldensis | Legionella lytica | Legionella shakespearei | |
| Legionella busanensis | Legionella fallonii | Legionella maceachernii | Legionella spiritensis | |
| Legionella cardiaca | Legionella feeleii | Legionella massiliensis | Legionella steelei | |
| Legionella cherrii | Legionella geestiana | Legionella micdadei | Legionella steigerwaltii | |
| Legionella cincinnatiensis | Legionella genomospecies | Legionella monrovica | Legionella taurinensis | |
| Legionella clemsonensis | Legionella gormanii | Legionella moravica | Legionella tucsonensis | |
| Legionella donaldsonii | Legionella gratiana | Legionella nagasakiensis | Legionella tunisiensis | |
| | Legionella gresilensis | Legionella nautarum | Legionella wadsworthii | |
| | Legionella hackeliae | Legionella norrlandica | Legionella waltersii | |
| | Legionella impletisoli | Legionella oakridgensis | Legionella worsleiensis | |
| | Legionella israelensis | Legionella parisiensis | Legionella yabuuchiae | |
| | Legionella jamestowniensis | Legionella pittsburghensis | | |
| | | Legionella pneumophila | | |
| | | Legionella quateirensis | | |
| Oceanibulbus | Paenibacillus | Prevotella | Quadrisphaera | |
| Oceanibulbus indolifex | Paenibacillus thiaminolyticus | Prevotella albensis | Quadrisphaera | |
| Oceanicaulis | Pantoea | Prevotella amnii | granulorum | |
| Oceanicaulis alexandrii | Pantoea | Prevotella bergensis | Quatrionicoccus | |
| Oceanicola | agglomerans | Prevotella bivia | Quatrionicoccus | |
| Oceanicola batsensis | Paracoccus | Prevotella brevis | australiensis | |
| Oceanicola granulosus | Paracoccus alcaliphilus | Prevotella bryantii | Quinella | |
| Oceanicola nanhaiensis | Paucimonas | Prevotella buccae | Quinella | |
| Oceanimonas | Paucimonas lemoignei | Prevotella buccalis | ovalis | |
| Oceanimonas baumannii | Pectobacterium | Prevotella copri | Ralstonia | |
| Oceaniserpentilla | Pectobacterium aroidearum | Prevotella dentalis | Ralstonia eutropha | |
| Oceaniserpentilla haliotis | Pectobacterium atrosepticum | Prevotella denticola | Ralstonia insidiosa | |
| Oceanisphaera | Pectobacterium betavasculorum | Prevotella disiens | Ralstonia mannitolilytica | |
| Oceanisphaera donghaensis | Pectobacterium cacticida | Prevotella histicola | Ralstonia pickettii | |
| Oceanisphaera litoralis | Pectobacterium carnegieana | Prevotella intermedia | Ralstonia | |
| Oceanithermus | Pectobacterium carotovorum | Prevotella maculosa | pseudosolanacearum | |
| Oceanithermus desulfurans | Pectobacterium chrysanthemi | Prevotella marshii | Ralstonia syzygii | |
| Oceanithermus profundus | Pectobacterium cypripedii | Prevotella melaninogenica | Ralstonia solanacearum | |
| Oceanobacillus | Pectobacterium rhapontici | Prevotella micans | Ramlibacter | |
| Oceanobacillus caeni | Pectobacterium wasabiae | Prevotella multiformis | Ramlibacter henchirensis | |
| Oceanospirillum | Planococcus | Prevotella nigrescens | Ramlibacter | |
| Oceanospirillum linum | Planococcus citreus | Prevotella oralis | tataouinensis | |
| | Planomicrobium | Prevotella oris | Raoultella | |
| | Planomicrobium okeanokoites | Prevotella oulorum | Raoultella ornithinolytica | |
| | Plesiomonas | Prevotella pallens | Raoultella planticola | |
| | Plesiomonas shigelloides | Prevotella salivae | Raoultella terrigena | |
| | Proteus | Prevotella stercorea | Rathayibacter | |
| | Proteus vulgaris | Prevotella tannerae | Rathayibacter caricis | |
| | | Prevotella timonensis | Rathayibacter festucae | |
| | | Prevotella veroralis | Rathayibacter iranicus | |
| | | Providencia | Rathayibacter rathayi | |
| | | Providencia stuartii | Rathayibacter toxicus | |
| | | Pseudomonas | Rathayibacter tritici | |
| | | Pseudomonas aeruginosa | Rhodobacter | |
| | | Pseudomonas alcaligenes | Rhodobacter sphaeroides | |
| | | Pseudomonas anguillispetica | Ruegeria | |
| | | Pseudomonas fluorescens | Ruegeria gelatinovorans | |
| | | Pseudoalteromonas | | |
| | | haloplanktis | | |
| | | Pseudomonas mendocina | | |
| | | Pseudomonas | | |
| | | pseudoalcaligenes | | |
| | | Pseudomonas putida | | |
| | | Pseudomonas tutzeri | | |
| | | Pseudomonas syringae | | |
| | | Psychrobacter | | |
| | | Psychrobacter faecalis | | |
| | | Psychrobacter | | |
| | | phenylpyruvicus | | |
| Saccharococcus | Sagittula | Sanguibacter | Stenotrophomonas | Tatlockia |
| Saccharococcus thermophilus | Sagittula stellata | Sanguibacter keddieii | Stenotrophomonas | Tatlockia maceachernii |
| Saccharomonospora | Salegentibacter | Sanguibacter suarezii | maltophilia | Tatlockia micdadei |
| Saccharomonospora azurea | Salegentibacter salegens | Saprospira | Streptococcus | Tenacibaculum |
| Saccharomonospora cyanea | Salimicrobium | Saprospira grandis | [also see below] | Tenacibaculum |
| Saccharomonospora viridis | Salimicrobium album | Sarcina | Streptomyces | amylolyticum |
| Saccharophagus | Salinibacter | Sarcina maxima | Streptomyces | Tenacibaculum discolor |
| Saccharophagus degradans | Salinibacter ruber | Sarcina ventriculi | achromogenes | Tenacibaculum |
| Saccharopolyspora | Salinicoccus | Sebaldella | Streptomyces | gallaicum |
| Saccharopolyspora erythraea | Salinicoccus alkaliphilus | Sebaldella | cesalbus | Tenacibaculum |
| Saccharopolyspora gregorii | Salinicoccus hispanicus | termitidis | Streptomyces cescaepitosus | lutimaris |
| Saccharopolyspora hirsuta | Salinicoccus roseus | Serratia | Streptomyces cesdiastaticus | Tenacibaculum |
| Saccharopolyspora hordei | Salinispora | Serratia fonticola | Streptomyces cesexfoliatus | mesophilum |
| Saccharopolyspora rectivirgula | Salinispora arenicola | Serratia marcescens | Streptomyces fimbriatus | Tenacibaculum |
| Saccharopolyspora spinosa | Salinispora tropica | Sphaerotilus | Streptomyces fradiae | skagerrakense |
| Saccharopolyspora taberi | Salinivibrio | Sphaerotilus natans | Streptomyces fulvissimus | Tepidanaerobacter |
| Saccharothrix | Salinivibrio costicola | Sphingobacterium | Streptomyces griseoruber | Tepidanaerobacter |
| Saccharothrix australiensis | Salmonella | Sphingobacterium multivorum | Streptomyces griseus | syntrophicus |
| Saccharothrix coeruleofusca | Salmonella bongori | Staphylococcus | Streptomyces lavendulae | Tepidibacter |
| Saccharothrix espanaensis | Salmonella enterica | [see below] | Streptomyces | Tepidibacter |
| Saccharothrix longispora | Salmonella subterranea | | phaeochromogenes | formicigenes |
| Saccharothrix mutabilis | Salmonella typhi | | Streptomyces | Tepidibacter thalassicus |
| Saccharothrix syringae | | | thermodiastaticus | Thermus |
| Saccharothrix tangerinus | | | Streptomyces tubercidicus | Thermus aquaticus |
| Saccharothrix texasensis | | | | Thermus filiformis |
| | | | | Thermus thermophilus |
| Staphylococcus | | | | |
| S. arlettae | S. equorum | S. microti | S. schleiferi | |
| S. agnetis | S. felis | S. muscae | S. sciuri | |
| S. aureus | S. fleurettii | S. nepalensis | S. simiae | |
| S. auricularis | S. gallinarum | S. pasteuri | S. simulans | |
| S. capitis | S. haemolyticus | S. petrasii | S. stepanovicii | |
| S. caprae | S. hominis | S. pettenkoferi | S. succinus | |
| S. carnosus | S. hyicus | S. piscifermentans | S. vitulinus | |
| S. caseolyticus | S. intermedius | S. pseudintermedius | S. warneri | |
| S. chromogenes | S. kloosii | S. pseudolugdunensis | S. xylosus | |
| S. cohnii | S. leei | S. pulvereri | | |
| S. condimenti | S. lentus | S. rostri | | |
| S. delphini | S. lugdunensis | S. saccharolyticus | | |
| S. devriesei | S. lutrae | S. saprophyticus | | |
| S. epidermidis | S. lyticans | | | |
| | S. massiliensis | | | |
| Streptococcus | | | | |
| Streptococcus agalactiae | Streptococcus infantarius | Streptococcus orisratti | Streptococcus thermophilus | |
| Streptococcus anginosus | Streptococcus iniae | Streptococcus parasanguinis | Streptococcus sanguinis | |
| Streptococcus bovis | Streptococcus intermedius | Streptococcus peroris | Streptococcus sobrinus | |
| Streptococcus canis | Streptococcus lactarius | Streptococcus pneumoniae | Streptococcus suis | |
| Streptococcus constellatus | Streptococcus milleri | Streptococcus | Streptococcus uberis | |
| Streptococcus downei | Streptococcus mitis | pseudopneumoniae | Streptococcus vestibularis | |
| Streptococcus dysgalactiae | Streptococcus mutans | Streptococcus pyogenes | Streptococcus viridans | |
| Streptococcus equines | Streptococcus oralis | Streptococcus ratti | Streptococcus | |
| Streptococcus faecalis | Streptococcus tigurinus | Streptococcus salivariu | zooepidemicus | |
| Streptococcus ferus | | | | |
| Uliginosibacterium | Vagococcus | Vibrio | Virgibacillus | Xanthobacter |
| Uliginosibacterium | Vagococcus carniphilus | Vibrio aerogenes | Virgibacillus | Xanthobacter agilis |
| gangwonense | Vagococcus elongatus | Vibrio aestuarianus | halodenitrificans | Xanthobacter |
| Ulvibacter | Vagococcus fessus | Vibrio albensis | Virgibacillus | aminoxidans |
| Ulvibacter litoralis | Vagococcus fluvialis | Vibrio alginolyticus | pantothenticus | Xanthobacter |
| Umezawaea | Vagococcus lutrae | Vibrio campbellii | Weissella | autotrophicus |
| Umezawaea tangerina | Vagococcus salmoninarum | Vibrio cholerae | Weissella cibaria | Xanthobacter flavus |
| Undibacterium | Variovorax | Vibrio cincinnatiensis | Weissella confusa | Xanthobacter tagetidis |
| Undibacterium pigrum | Variovorax boronicumulans | Vibrio coralliilyticus | Weissella halotolerans | Xanthobacter viscosus |
| Ureaplasma | Variovorax dokdonensis | Vibrio cyclitrophicus | Weissella hellenica | Xanthomonas |
| Ureaplasma | Variovorax paradoxus | Vibrio diazotrophicus | Weissella kandleri | Xanthomonas |
| urealyticum | Variovorax soli | Vibrio fluvialis | Weissella koreensis | albilineans |
| Ureibacillus | Veillonella | Vibrio furnissii | Weissella minor | Xanthomonas alfalfae |
| Ureibacillus composti | Veillonella atypica | Vibrio gazogenes | Weissella | Xanthomonas |
| Ureibacillus suwonensis | Veillonella caviae | Vibrio halioticoli | paramesenteroides | arboricola |
| Ureibacillus terrenus | Veillonella criceti | Vibrio harveyi | Weissella soli | Xanthomonas |
| Ureibacillus thermophilus | Veillonella dispar | Vibrio ichthyoenteri | Weissella thailandensis | axonopodis |
| Ureibacillus thermosphaericus | Veillonella montpellierensis | Vibrio mediterranei | Weissella viridescens | Xanthomonas |
| | Veillonella parvula | Vibrio metschnikovii | Williamsia | campestris |
| | Veillonella ratti | Vibrio mytili | Williamsia marianensis | Xanthomonas citri |
| | Veillonella rodentium | Vibrio natriegens | Williamsia maris | Xanthomonas codiaei |
| | Venenivibrio | Vibrio navarrensis | Williamsia serinedens | Xanthomonas |
| | Venenivibrio stagnispumantis | Vibrio nereis | Winogradskyella | cucurbitae |
| | Verminephrobacter | Vibrio nigripulchritudo | Winogradskyella | Xanthomonas |
| | Verminephrobacter eiseniae | Vibrio ordalii | thalassocola | euvesicatoria |
| | Verrucomicrobium | Vibrio orientalis | Wolbachia | Xanthomonas fragariae |
| | Verrucomicrobium spinosum | Vibrio parahaemolyticus | Wolbachia persica | Xanthomonas fuscans |
| | | Vibrio pectenicida | Wolinella | Xanthomonas gardneri |
| | | Vibrio penaeicida | Wolinella succinogenes | Xanthomonas hortorum |
| | | Vibrio proteolyticus | Zobellia | Xanthomonas hyacinthi |
| | | Vibrio shilonii | Zobellia galactanivorans | Xanthomonas perforans |
| | | Vibrio splendidus | Zobellia uliginosa | Xanthomonas phaseoli |
| | | Vibrio tubiashii | Zoogloea | Xanthomonas pisi |
| | | Vibrio vulnificus | Zoogloea ramigera | Xanthomonas populi |
| | | | Zoogloea resiniphila | Xanthomonas theicola |
| | | | | Xanthomonas |
| | | | | translucens |
| | | | | Xanthomonas |
| | | | | vesicatoria |
| | | | | Xylella |
| | | | | Xylella fastidiosa |
| | | | | Xylophilus |
| | | | | Xylophilus ampelinus |
| Xenophilus | Yangia | Yersinia mollaretii | Zooshikella | Zobellella |
| Xenophilus azovorans | Yangia pacifica | Yersinia philomiragia | Zooshikella ganghwensis | Zobellella denitrificans |
| Xenorhabdus | Yaniella | Yersinia pestis | Zunongwangia | Zobellella taiwanensis |
| Xenorhabdus beddingii | Yaniella flava | Yersinia pseudotuberculosis | Zunongwangia profunda | Zeaxanthinibacter |
| Xenorhabdus bovienii | Yaniella halotolerans | Yersinia rohdei | Zymobacter | Zeaxanthinibacter |
| Xenorhabdus cabanillasii | Yeosuana | Yersinia ruckeri | Zymobacter palmae | enoshimensis |
| Xenorhabdus doucetiae | Yeosuana aromativorans | Yokenella | Zymomonas | Zhihengliuella |
| Xenorhabdus griffiniae | Yersinia | Yokenella regensburgei | Zymomonas mobilis | Zhihengliuella |
| Xenorhabdus hominickii | Yersinia aldovae | Yonghaparkia | Zymophilus | halotolerans |
| Xenorhabdus koppenhoeferi | Yersinia bercovieri | Yonghaparkia alkaliphila | Zymophilus paucivorans | Xylanibacterium |
| Xenorhabdus nematophila | Yersinia enterocolitica | Zavarzinia | Zymophilus raffinosivorans | Xylanibacterium ulmi |
| Xenorhabdus poinarii | Yersinia entomophaga | Zavarzinia compransoris | | |
| Xylanibacter | Yersinia frederiksenii | | | |
| Xylanibacter oryzae | Yersinia intermedia | | | |
| | Yersinia kristensenii | | | |

1. A composition for use in antibacterial treatment of bacteria, the composition comprising an engineered mobile genetic element (MGE) that is capable of being mobilised in a first bacterial host cell of a first species or strain, the cell comprising a first phage genome, wherein in the cell the MGE is mobilised using proteins encoded by the phage and replication of first is inhibited, wherein the MGE encodes an antibacterial agent or encodes a component of such an agent.

2. The composition of claim 1, wherein the agent is toxic to cells of the same species or strain as the host cell.

3. The composition of claim 1 or 2, wherein the agent is toxic to cells of a species or strain that is different from the strain or species of the host cell.

4. The composition of claim 1, wherein the agent is toxic to cells of the same species as the host cell, and wherein the host cell has been engineered so that the agent is not toxic to the host cell.

5. The composition of claim 4, wherein the agent is a guided nuclease system (optionally a CRISPR/Cas system) and cells of the same species as the host cell comprise a target sequence that is cut by the nuclease, wherein the target sequence has been removed or altered in the host cell whereby the nuclease is not capable of cutting the target sequence.

6. The composition of any preceding claim, wherein the first phage is a temperate phage.

7. The composition of any preceding claim, wherein the first cell comprises the first phage as a prophage.

8. The composition of any one of claims 1 to 5, wherein the first phage is a lytic phage.

9. The composition of any preceding claim, wherein in the presence of a first phage the mobilisation of the MGE causes host cell lysis.

10. The composition of any preceding claim, wherein the MGE is capable of being packaged in transduction particles that comprise some, but not all, structural proteins of the first phage.

11. The composition of any preceding claim, wherein mobilisation of the MGE comprises packaging of copies of the MGE or nucleic acid encoding the agent or component into transduction particles that are capable of transferring the copies into target bacterial cells for antibacterial treatment of the target cells.

12. The composition of claim 10 or 11, wherein the transduction particles are particles of second phage that are capable of infecting cells of said first species or strain.

13. The composition of any one of claims 10 to 12, wherein the transduction particles are non-self replicative particles.

14. The composition of any preceding claim, wherein the MGE is devoid of genes encoding phage structural proteins.

15. The composition of any one of claims 1 to 13, wherein the MGE comprises phage structural genes and a packaging signal sequence and the first phage is devoid of a packaging signal sequence.

16. The composition of any preceding claim, wherein the MGE is a modified version of a MGE that is naturally found in bacterial cells of the first species or strain.

17. The composition of any preceding claim, wherein the MGE comprises a modified genomic island.

18. The composition of any preceding claim, wherein the MGE comprises a modified pathogenicity island.

19. The composition of claim 18, wherein the pathogenicity island is a SaPI (S aureus pathogenicity island).

20. The composition of claim 19, wherein the first phage is ϕ11, 80α, ϕ12 or ϕSLT.

21. The composition of claim 18, wherein the pathogenicity island is a V. cholerae PLE (phage-inducible chromosomal island-like element) and optionally the first phage is ICP1.

22. The composition of claim 18, wherein the pathogenicity island is a E coli PLE.

23. The composition of any one of claims 1 to 16, wherein the MGE comprises P4 DNA, eg, a P4 packaging signal sequence.

24. The composition of claim 23, wherein the first phage are P2 phage or a modified P2 phage that is self-replicative defective; optionally present as a prophage.

25. The composition of any preceding claim, wherein the MGE comprises a pacA gene of the Enterobacteriaceae bacteriophage P1.

26. The composition of any preceding claim, wherein the MGE comprises a packaging initiation site sequence, optionally a packaging initiation site sequence of P1.

27. The composition of any preceding claim, wherein the MGE comprises a nucleotide sequence that is beneficial to cells of the first species or strain, optionally encoding a protein that is beneficial to cells of the first species or strain.

28. The composition of any preceding claim, wherein the MGE is devoid of rinA.

29. The composition of any preceding claim, wherein the MGE is is devoid of terL.

30. The composition of any preceding claim, wherein the MGE comprises a terS or a homologue thereof, and optionally is devoid of any other terminase gene.

31. The composition of any preceding claim, wherein the first phage is a pac-type phage operable with a pac comprised by the MGE.

32. The composition of any one of claims 1 to 30, wherein the first phage is a cos-type phage operable with a cos comprised by the MGE.

33. The composition of any preceding claim, wherein the plasmid or MGE comprises a pac and/or cos sequence or a homologue thereof.

34. The composition of any preceding claim, wherein the plasmid or MGE comprises a terS or a homologue thereof and optionally devoid of terL.

35. The composition of claim 34, wherein the terS is a S aureus bacteriophage φ80α terS or a bacteriophage φ11 terS.

36. The composition of any preceding claim, wherein the MGE is a modified SaPIbov1 or SaPIbov5 and is devoid of a terS.

37. The composition of any preceding claim, wherein the first phage is devoid of a functional packaging signal sequence and the MGE comprises a packaging signal sequence operable with proteins encoded by the first phage for producing transduction particles that package copies of the MGE or copies of a nucleic acid encoding the agent or component.

38. The composition of any preceding claim, wherein the MGE or plasmid comprises a Ppi or homologue, which is capable of complexing with first phage TerS, thereby blocking function of the TerS.

39. The composition of any preceding claim, wherein the MGE comprises a morphogenesis (cpm) module.

40. The composition of any preceding claim, wherein the MGE comprises cpmA and/or cpmB.

41. The composition of any preceding claim, wherein the MGE or first phage comprises one, more or all genes cp1, cp2, and cp3.

42. The composition of any preceding claim, wherein the MGE or first phage encodes a HNH nuclease.

43. The composition of any preceding claim, wherein the MGE or first phage comprises an integrase gene that encodes an integrase for excising the MGE and integrating the MGE into a bacterial cell genome.

44. The composition of any preceding claim, wherein the MGE is devoid of a functional integrase gene, and the first phage or host cell genome (eg, bacterial chromosome or a bacterial episome) comprises a functional integrase gene.

45. The composition of any preceding claim, wherein the transcription of MGE nucleic acid is under the control of a constitutive promoter, for transcription of copies of the agent or component in a host cell.

46. The composition of claim 45, wherein the promoter is foreign to the host cell.

47. The composition of claim 45 or 46, wherein the promoter comprises a nucleotide sequence that is at least 80% identical to an endogenous promoter sequence of the host cell.

48. The composition of any preceding claim comprising a nucleic acid that is separate from the MGE, wherein the nucleic acid comprises all genes necessary for producing first phage particles.

49. The composition of any one of claims 1 to 47 comprising a nucleic acid that is separate from the MGE, wherein the nucleic acid comprises less than, all genes necessary for producing first phage particles, but comprises genes encoding structural proteins for production of transduction particles that package MGE nucleic acid encoding the antibacterial agent or one or more components thereof.

50. The composition of claim 48 or 49, wherein the genes are comprised by the host cell chromosome and/or one or more host cell episome(s).

51. The composition of claim 50, wherein the genes are comprised by a chromosomally-integrated prophage of the first phage.

52. The composition of any preceding claim, wherein the agent is a guided nuclease system or a component thereof, wherein the agent is capable of recognising and cutting host cell DNA (eg, chromosomal DNA).

53. The composition of claim 52, wherein the guided nuclease system is selected from a CRISPR/Cas system, TALEN system, meganuclease system or zinc finger system.

54. The composition of claim 52, wherein the system is a CRISPR/Cas system and each MGE encodes a (a) CRISPR array encoding crRNA or (b) a nucleic acid encoding a guide RNA (gRNA, eg, single guide RNA), wherein the crRNA or gRNA is operable with a Cas in target bacterial cells, wherein the crRNA or gRNA guides the Cas to a target nucleic acid sequence in the host cell to modify the target sequence (eg, cut it or repress transcription from it).

55. The composition of claim 52, wherein the system is a CRISPR/Cas system and each MGE encodes a Cas (eg, a Cas nuclease) that is operable in a target bacterial cells to modify a target nucleic acid sequence comprised by the target cell.

56. The composition of claim 53, 54 or 55, wherein the Cas is a Cas3, Cas9, Cas13, CasX, CasY or Cpf1.

57. The composition of any one of claims 52 to 56, wherein the system is a CRISPR/Cas system and each MGE encodes one or more Cascade Cas (eg, Cas, A, B, C, D and E).

58. The composition of any one of claims 52 to 57, wherein each MGE further encodes a Cas3 that is operable in a target bacterial cell with the Cascade Cas.

59. The composition of any preceding claim, wherein the first species or strain is a gram positive species or strain.

60. The composition of any one of claims 1 to 58, wherein the first species or strain is a gram negative species or strain.

61. The composition of any preceding claim, wherein the first species or strain is selected from Table 1.

62. The composition of any preceding claim, wherein the first species or strain is selected from Shigella, E coli, Salmonella, Serratia, Klebsiella, Yersinia, Pseudomonas and Enterobacter.

63. A nucleic acid vector comprising a MGE integrated therein, wherein the MGE is according to any preceding claim and the vector is capable of transferring the MGE or a copy thereof into a host bacterial cell.

64. The vector of claim 63, wherein the vector is a shuttle vector.

65. The vector of claim 63, wherein the vector is a plasmid, wherein the plasmid is capable of being transformed into a host bacterial cell comprising a first phage.

66. A non-self replicative transduction particle comprising said MGE or vector of any preceding claim.

67. A composition comprising a plurality of transduction particles, wherein each particle comprises a MGE or vector according to any one of claims 1 to 65, wherein the transduction particles are capable of transferring the MGEs, or nucleic acid encoding the agent or component, or copies thereof into target bacterial cells, wherein
(i) target cells are killed by the antibacterial agent;
(ii) growth or proliferation of target cells is reduced; or
(iii) target cells are sensitised to an antibiotic, whereby the antibiotic is toxic to the cells.

68. The composition of claim 67, wherein the agent is a guided nuclease system or a component thereof, wherein the agent is capable of recognising and cutting host cell DNA (eg, chromosomal DNA) whereby
(i) target cells are killed by the antibacterial agent;
(ii) growth or proliferation of target cells is reduced; or
(iii) target cells are sensitised to an antibiotic, whereby the antibiotic is toxic to the cells.

69. A composition comprising a plurality of non-self replicative transduction particles, wherein each particle comprises a MGE or plasmid according to any one of claims 1 to 65, wherein the transduction particles are capable of transferring the MGEs, or nucleic acid encoding the agent or component, or copies thereof into target bacterial cells, wherein the agent is a CRISPR/Cas system and the component comprises a nucleic acid encoding a crRNA or a guide RNA that is operable with a Cas in a target bacterial cell to guide the Cas to a target nucleic acid sequence of the cell to modify the sequence, whereby
(i) target cells are killed by the antibacterial agent;
(ii) growth or proliferation of target cells is reduced; or
(iii) target cells are sensitised to an antibiotic, whereby the antibiotic is toxic to the cells.

70. A kit comprising the composition of claim 69 and said antibiotic.

71. The composition of claim 69, wherein the composition comprises said antibiotic.

72. The composition of any one of claims 67 to 69, wherein less than 10% of transduction particles comprise by the composition are first phage particles.

73. The composition of any one of claims 67 to 69, wherein no first phage particles are present in the composition.

74. The MGE, vector, particle, composition or kit of any preceding claim for medical use in a human or animal patient.

75. The MGE, vector, particle, composition or kit of any preceding claim for treating or preventing an infection by target bacterial cells in a human or animal patient, wherein the antibacterial agent is toxic to the target cells.

76. The MGE, vector, particle, composition or kit of any preceding claim for treating or preventing an infection by target bacterial cells in a human or animal patient, wherein in the presence of the antibacterial agent
(i) target cells are killed by the antibacterial agent;
(ii) growth or proliferation of target cells is reduced; and/or
(iii) target cells are sensitised to an antibiotic, whereby the antibiotic is toxic to the cells.

77. A method of producing a plurality of transduction particles, the method comprising combining the composition of any one of claims 1 to 62, 67 to 69 and 71 to 76 with host bacterial cells of said first species, wherein the cells comprise the first phage, allowing a plurality of said MGEs to be introduced into host cells and culturing the host cells under conditions in which first phage-encoded proteins are expressed and MGE copies are packaged by first phage proteins to produce a plurality of transduction particles, and optionally separating the transduction particles from cells and obtaining a plurality of transduction particles separated from cells.

78. The method of claim 77, comprising separating the transduction particles from any first phage, optionally by filtering or centrifugation, thereby obtaining a plurality of transduction particles in the absence of first phage.

79. The method of claim 77 or 78, wherein the particles encode a guided nuclease system (optionally a CRISPR/Cas system) or component thereof for cutting a target nucleic acid sequence comprised by target bacterial cells.

80. The method of claim 79, wherein the sequence is comprised by an antibiotic resistance gene and the method comprises combining the plurality of particles with said antibiotic in a kit or a mixture.

81. The method of any one of claims 77 to 80, wherein said conditions comprise induction of a lytic cycle of the first phage.

82. A bacterial host cell comprising a first phage and a MGE, vector or particle as recited in any one of claims 1 to 66, wherein the agent is toxic to cells of the same species as the host cell, and wherein the host cell has been engineered so that the agent is not toxic to the host cell.

83. A bacterial host cell comprising a first phage, wherein the cell is comprised by a kit, the kit further comprising a composition as recited in any one of claims 1 to 62, 67 to 69 and 71 to 76, wherein the agent is toxic to cells of the same species as the host cell, and wherein the host cell has been engineered so that the agent is not toxic to the host cell.

84. The cell of claim 83, wherein the agent is a guided nuclease system (optionally a CRISPR/Cas system) and cells of the same species as the host cell comprise a target sequence that is cut by the nuclease, wherein the target sequence has been removed or altered in the host cell whereby the nuclease is not capable of cutting the target sequence.

85. A bacterial host cell comprising a first phage and a MGE, vector or particle as recited in any one of claims 1 to 66, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

86. A bacterial host cell comprising a first phage, wherein the cell is comprised by a kit, the kit further comprising a composition as recited in any one of claims 1 to 62, 67 to 69 and 71 to 76, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

87. The cell of claim 86, wherein the first phage is a prophage.

88. A bacterial host cell comprising a MGE, vector or particle as recited in any one of claims 1 to 66 and nucleic acid under the control of one or more inducible promoters, wherein the nucleic acid encodes all structural proteins necessary to produce a transduction particle that packages a copy of the MGE or plasmid, wherein the agent is not toxic to the host cell, but the agent is toxic to second cells of a species or strain that is different from the species or strain of the host cell, wherein the MGE is mobilizable in transduction particles producible by the host cell that are capable of transferring the MGE or a copy thereof into a said second cell, whereby the second cell is exposed to the antibacterial agent.

89. The cell of claim 88, wherein the structural proteins are structural proteins of a lytic phage.

90. The cell of claim 88 or 89, wherein the nucleic acid comprises terS and/or terL.

91. The cell of any one of claims 88 to 90, wherein the host and second cells are of the same species and the host cell has been engineered so that the antibiotic is not toxic to the host cell.

92. The cell of any one of claims 88 to 91, wherein the nucleic acid is comprised by a plasmid.

93. The cell of any one of claims 88 to 92, wherein the agent is a guided nuclease system (optionally a CRISPR/Cas system) and the second cells comprise a target sequence that is cut by the nuclease, wherein the target sequence is absent in the genome of the host cell whereby the nuclease is not capable of cutting the host cell genome.

94. The composition, vector, particle, kit or method of any preceding claim, wherein the cell, host cell or target cell is selected from a Staphylococcal, Vibrio, Pseudomonas, Clostridium, E coli, Helicobacter, Klebsiella and Salmonella cell.

95. A plasmid comprising
(i) A nucleotide sequence encoding an antibacterial agent or component thereof for expression in target bacterial cells;
(ii) A constitutive promoter for controlling the expression of the agent or component;
(iii) An optional terS nucleotide sequence;
(iv) An origin of replication (ori); and
(v) A phage packaging sequence (optionally pac, cos or a homologue thereof); and
the plasmid being devoid of
(vi) All nucleotide sequences encoding phage structural proteins necessary for the production of a transduction particle (optionally a phage), or the plasmid being devoid of at least one of such sequences; and
(vii) Optionally terL.

96. The plasmid of claim 95, wherein the antibacterial agent is a CRISPR/Cas system and the plasmid encodes a crRNa or guide RNA (eg, single gRNA) that is operable with a Cas in the target cells to guide the Cas to a target nucleotide sequence to modify (eg, cut) the sequence, whereby
(i) target cells are killed by the antibacterial agent;
(ii) growth or proliferation of target cells is reduced; or
(iii) target cells are sensitised to an antibiotic, whereby the antibiotic is toxic to the cells.

97. The plasmid of claim 95 or 96, wherein the antibacterial agent is a CRISPR/Cas system and the plasmid encodes a Cas that is operable with a crRNa or guide RNA (eg, single gRNA) in the target cells to guide the Cas to a target nucleotide sequence to modify (eg, cut) the sequence, whereby
(i) target cells are killed by the antibacterial agent;
(ii) growth or proliferation of target cells is reduced; or
(iii) target cells are sensitised to an antibiotic, whereby the antibiotic is toxic to the cells.

98. The plasmid of claim 97, wherein the plasmid further encodes said crRNA or gRNA.

99. A host cell comprising the plasmid of any one of claims 95 to 98, wherein the host cell does not comprise the target nucleotide sequence.

100. The host cell of claim 99, wherein the cell is capable of replicating the plasmid and packaging the replicated plasmid in transduction particles that are capable of infecting target bacterial cells.

101. The host cell of claim 99 or 100, wherein the host cell comprises, integrated in the cell chromosome and/or one or more episomes of the cell,
(i) A terL;
(ii) An optional terS; and
(iii) Expressible nucleotide sequences encoding all structural proteins necessary for the production of transduction particles that package copies of the plasmid;
wherein the chromosome and episomes of the cell (other than said plasmid) are devoid of a phage packaging sequence, wherein the phage packaging sequence comprised by the plasmid is operable together with the product of said terS and terL in the production of packaged plasmid.

102. The cell of claim 101, wherein the terL, optional terS and nucleotide sequences encoding the structural proteins are comprised by a phage (optionally a prophage) genome in the host cell.

103. A bacterial host cell comprising the genome of a helper phage that is incapable of self-replication, optionally wherein the genome is present as a prophage, and a plasmid according to any one of claims 95 to 98, wherein the helper phage is operable to package copies of the plasmid in transduction particles, wherein the particles are capable of infecting bacterial target cells to which the antibacterial agent is toxic.

104. The cell of claim 103, wherein the host cell is a cell of first species or strain and the target cells are of the same species or strain, and optionally wherein the hosts cell is an engineered cell that to which the antibacterial agent is not toxic.

105. The cell of claim 103, wherein the host cell is a cell of first species or strain and the target cells are of a different species or strain, wherein the antibacterial agent is not toxic to the host cell.

106. A method of making a plurality of transduction particles, the method comprising culturing a plurality of host cells according to any one of claims 103 to 105, optionally inducing a lytic cycle of the helper phage, and incubating the cells under conditions wherein transducing particles comprising packaged copies of the plasmid are created, and optionally separating the particles from the cells to obtain a plurality of transduction particles.

107. A plurality of transduction particles obtainable by the method of claim 106 for use in medicine, eg, for treating or preventing an infection of a human or animal subject by target bacterial cells, wherein transducing particles are administered to the subject for infecting target cells and killing the cells using the antibacterial agent.

108. A method of making a plurality of transduction particles, the method comprising
(i) Producing host cells whose genomes comprise nucleic acid encoding structural proteins necessary to produce transduction particles that can package first DNA, wherein the genomes are devoid of a phage packaging signal, wherein the expression of the proteins is under the control of inducible promoter(s);
(ii) Producing first DNA encoding an antibacterial agent or a component thereof (eg, as defined in any preceding claim), wherein the DNA comprises a phage packaging signal;
(iii) Introducing the DNA into the host cells;
(iv) Inducing production of the structural proteins in host cells, whereby transduction particles are produced that package the DNA;
(v) Optionally isolating a plurality of the transduction particles; and
(vi) Optionally formulating the particles into a pharmaceutical composition for administration to a human or animal for medical use.

109. The method of claim 108, wherein the DNA comprises a MGE as defined in any preceding claim.

110. The method of claim 108 or 109, wherein the structural proteins are P2 phage proteins and optionally the packaging signal is a P4 phage packaging signal.

111. The method of claim 108 or 109, wherein the DNA comprises a modified SaPI or a genomic island DNA.

112. The method of any one of claims 108 to 111, wherein the cells in step (iv) comprise a gene encoding a helper phage activator, optionally wherein the activator is a P4 phage delta or ogr protein when the structural proteins are P2 proteins; or the activator is a SaPI rinA, ptiA, ptiB or ptiM when the MGE comprises a modified SaPI; and optionally the expression of the activator(s) is controlled by an inducible promoter, eg, a T7 promoter.

113. The method of any one of claims 108 to 112, wherein the packaging signal is P4 phage Sid and/or psu; or the signal is SaPI cpmA and/or cpmB.

114. The method of any one of claims 108 to 113, wherein the cell genomes comprise prophages, wherein each prophage comprises said nucleic acid encoding structural proteins.

115. The method of claim 114, wherein the prophages are P2 prophages devoid of cos and optionally one, more or all genes selected from int, cox orf78, B, orf80, orf81, orf82, orf83, A, orf91, tin, old, orf30 and fun(Z); and optionally the packaging signal of (ii) is a cos or P4 packaging signal.

116. The method of claim 114 or 115, wherein the prophages are P2 prophages devoid of cos and comprising genes from Q to S, V to G and F_{I} to ogr.

117. The method of claim 114, wherein the prophages are phi11 prophages devoid of a packaging signal and comprising gene 29 (terS) to gene 53 (lysin); and optionally the packaging signal of (ii) is a phi11 packaging signal.

118. A plurality of transduction particles obtainable by the method of any one of claims 108 to 117.

119. The particles of claim 118 for administration to a human or animal for medical use.


SEQUENCE LISTING (33 sequences)

SEQ ID NO: 1 - DNA, 325 bp, synthetic construct
  misc_feature 1..325: note = Synthetic Construct
  source 1..325: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaacccagaagttagttaattatttgtgtagtcaaagtgccttgactacatacctcgttaatacattggagcataatgaagaaaatctatggcctatggtccaaaactgtcttttttgatggcactatcctgaaaaatatgcaaaaaatagattgatgtaaggtggttcttgtcagtgtcgcaagatccttaagaattc

SEQ ID NO: 2 - DNA, 663 bp, synthetic construct
  misc_feature 1..663: note = Synthetic Construct
  source 1..663: mol_type = other DNA
aattggcagtaaagtggcagtttttgatacctaaaatgagatattatgatagtgtaggatattgactatcttactgcgtttcccttatcgcaattaggaataaaggatctatgtgggttggctgattatagccaatccttttttaattttaaaaagcgtatagcgcgagagttggtggtaaatgaaatgaacgaaaaacaaaagagattcgcagatgaatatataatgaatggatgtaatggtaaaaaagcagcaatttcagcaggttatagtaagaaaacagcagagtctttagcaagtcgattgttaagaaatgttaatgtttcggaatatattaaagaacgattagaacagatacaagaagagcgtttaatgagcattacagaagctttagcgttatctgcttctattgctagaggagaacctcaagaggcttacagtaagaaatatgaccatttaaacgatgaagtggaaaaagaggttacttacacaatcacaccaacttttgaagagcgtcagagatctattgaccacatactaaaagttcatggtgcgtatatcgacaaaaaagaaattactcagaagaatattgagattaatattggtgagtacgatgacgaaagttaaattaaactttaacaaaccatctaatgttttcaacag

SEQ ID NO: 3 - DNA, 227 bp, synthetic construct
  misc_feature 1..227: note = Synthetic Construct
  source 1..227: mol_type = other DNA
gcatgcgttttcctgcctcattttctgcaaaccgcgccattcccggcgcggtctgagcgtgtcagtgcaactgcattaaaaccgccccgcaaagcgggcgggcgaggcggggaaagcaccgcgcgcaaaccgacaagttagttaattatttgtgtagtcaaagtgccttcagtacatacctcgttaatacattggagcataatgaagaaaatctatggcctatggtc

SEQ ID NO: 4 - DNA, 55 bp, synthetic construct
  misc_feature 1..55: note = Synthetic Construct
  source 1..55: mol_type = other DNA
tgcattaaaaccgccccgcaaagcgggcgggcgaggcggggaaagcaccgcgcgc

SEQ ID NO: 5 - DNA, 735 bp, synthetic construct
  misc_feature 1..735: note = Synthetic Construct
  source 1..735: mol_type = other DNA
atgtctgaccacactatccctgaatatctgcaacccgcactggcacaactggaaaaggccagagccgcccatcttgagaacgcccgcctgatggatgagaccgtcacggccattgaacgggcagagcaggaaaaaaatgcgctggcgcaggccgacggaaacgacgctgacgactggcgcacggcctttcgtgcagccggtggtgtcctgagcgacgagctgaaacagcgccacattgagcgcgtggcacgccgggagctggtacaggaatatgacaatctggccgtggtgctgaatttcgaacgtgaacgcctgaaaggggcgtgtgacagcacggccaccgcctaccggaaggcacatcatcaccttctgagtctgtatgcagagcatgagctggaacacgccctgaatgaaacctgtgaggcgcttgtccgggcaatgcatctgagcattctggtacaggaaaatccgctcgccaacaccaccggccatcagggctacgtcgcaccggaaaaggctgtcatgcagcaggtgaaatcatcgctggaacagaaaattaaacagatgcaaatcagcctcaccggcgagccggttctccggctgaccggactgtcagcggcaacactcccgcacatggattatgaggtggcaggcacaccggcacagcgcaaggtgtggcaggacaaaatagaccagcagggagcagagcttaaggccagagggctgctgtcatga

SEQ ID NO: 6 - DNA, 573 bp, synthetic construct
  misc_feature 1..573: note = Synthetic Construct
  source 1..573: mol_type = other DNA
atggaaagcacagccttacagcaggcctttgacacctgtcagaataacaaagcagcatggctgcaacgcaaaaatgagctggcagcggccgaacaggaatatctgcggcttctgtcaggagaaggcagaaacgtcagtcgcctggacgaattacgcaatattatcgaagtcagaaaatggcaggtgaatcaggccgccggtcgttatattcgttcgcatgaagccgttcagcacatcagcatccgcgaccggctgaatgattttatgcagcagcacggcacagcactggcggccgcactggcaccggagctgatgggctacagtgagctgacggccattgcccgaaactgtgccatacagcgtgccacagatgccctgcgtgaagcccttctgtcctggcttgcgaagggtgaaaaaattaattattccgcacaggatagcgacattttaacgaccatcggattcaggcctgacgtggcttcggtggatgacagccgtgaaaaattcacccctgcgcagaacatgattttttcgcgtaaaagtgcgcaactggcatcacgtcagtcagtgtaa

SEQ ID NO: 7 - DNA, 501 bp, synthetic construct
  misc_feature 1..501: note = Synthetic Construct
  source 1..501: mol_type = other DNA
atgatttactgtccgtcgtgtggacatgttgctcacacccgtcgcgcacatttcatggacgatggcaccaagataatgattgcacagtgccggaatatttattgctctgcgacatttgaagcgagtgaaagctttttctctgacagtaaagattcaggaatggaatacatttcaggcaaacagagataccgcgattcactgacgtcagcctcctgcggtatgaaacgcccgaaaagaatgcttgttaccggatattgttgtcggagatgtaaaggccttgcactgtcaagaacatcgcggcgtctgtctcaggaagtcaccgagcgtttttatgtgtgcacggatccgggctgtggtctggtgtttaaaacgcttcagaccatcaaccgcttcattgtccgcccggtcacgccggacgaactggcagaacgcctgcatgaaaaacaggaactgccgccagtacggttaaaaacacaatcatattcgctgcgtctggaatga

SEQ ID NO: 8 - DNA, 9181 bp, synthetic construct
  misc_feature 1..9181: note = Synthetic Construct
  source 1..9181: mol_type = other DNA
tcagtcgttgtcagtgtccagtgagtagtttttaaagcggatgacctcctgaccgagccagccgtttatctcgcggatcctgtcctgtaacgggataagctcattgcggacaaagacctttgccactttctcaatatcacccagcgacccgacgttctccggcttgccacccatcaactgaaaggggatgcggtgcgcgtccagcaggtcagcggcgctggcttttttgatattaaaaaaatcgtccttcgtcgccacttcactgagggggataattttaatgccgtcggctttcccctgtggggcatagagaaacaggtttttaaagttgttgcggcctttcgacttgaccatgttttcgcgaagcatttcgatatcgttgcgatcctgcacggcatcggtgacatacatgatgtatccggcatgtgcgccattttcgtaatacttgcggcggaacaacgtggccgactcattcagccaggcagagttaagggcgctgagatattccggcaggccgtacagctcctgattaatatccggctccagcaggtgaaacacggagccgggcgcgaaggctgtcggctcgttgaaggacggcacccaccagtaaacatcctcttccacgccacggcgggtatattttgccggtgaggtttccagtctgatgaccttaccggtggtgctgtaacgcttttccagaaacgcattaccgaacaccagaaaatccagcacaaagcggctgaaatcctgctgggaaagccatggatgcgggataaatgtcgaggccagaatattgcgtttgacgtaaatcggcgagctgtgatgcacggcagcccgcaggctttttgccagaccggtaaagctgaccggtggctcataccatctgccgttactgatgcactcgacgtaatccagaatgtcacggcggtcgagtaccggcaccggctcaccaaaggtgaatgcctccattttcgggccgctggcggtcattgtttttgccgcaggttgcggtgttttcccttttttcttgctcatcagtaaaactccagaatggtggatgtcagcggggtgctgataccggcggtgagtggctcatttaacagggcgtgcatggtcgcccaggcgaggtcggcgtggctggcttcctcgctgcggctggcctcataggtggcgctgcgtccgctgctggtcatggtcttgcggatagccataaacgagctggtgatgtcggtggcgctgacgtcgtattccagacagccacggcggataacgtcttttgccttgagcaccattgcggttttcatttccggcgtgtagcggatatcacgcgcggcgggatagaacgagcgcacgagctggaacacgccgacaccgaggccggtggcatcaataccgatgtattcgacgttgtatttttcggtgagtttgcggatggattccgcctgggtggcaaagtccatgcctttccactggtgacgctcaagtattctgaatttgccaccggccaccaccggcggtgccagtaccacgcatccggcgctgtcgccacggtgtgacgggtcgtaaccaatccataccgggcgggagccgaacggattggcggcaaacggcgcatagtcttcccattcttccagcgtgtcgaccatgcagcgttgcagctcctcgaacgggaacaccgatgccttgtcgtcaacaaattcacacatgaacaggtttttaaaatcgtcggcgctgttttcgcgtttgagctgctcaatgtcgaacagcgtgcagccgcctttcagggcgtcctcaatggtgacaatctgtcgccactggccgtccgcacagagaagcccaccggcaagggcgttatgactgacgtcgatttccacgcgttcggcggcgctggcgcgtccccggttaaacagttcacccgaccagaacgggtaggcgtcgtgcgccagcgtggacggggtggagaaataggtcgagcgcaggtgactctgtgaggccatacctgatgccaccttacgcagtacctgaaaattcgggatccagaaaatctcatcgacgtacaggtcgccgttatgactctgcgcggtgttggagttggtgccgagaaaaatcagttttgcgccgttattgcccaggacaatcgggtcaccggtcaggtcaacgtcaaccagacgggcaaaggcgatgatgtattcgcggaacacatacgcctgtgttttactggccgacagaaaaatctggttatgaccggttttcagggcacgcagcagcgcctcgcgggaaaaataaaacgtcgcgccaatctggcgggatttcaggatatcgcggatgcggtgctcaagcccggcacgataccagtgcagctgatagtcgaaagactgctcaaagaaaatctgctccagcttttcgatggcctcgtcactgaaaaaattctttttcggtttgcgccgtccgcctttgttacggttagcgacgttcggattaaggtctgcctcgttgccggtctggctgtagcggttgacccgtgccagtcgttcaatctggcgtcccagcaggtcaatttccttgaagtcaccgccggttttctgtggtttgatgatgagctgggtcagccgcgcttccagactcatttcgacacggctgatgggggcaacgctgtcccagccgtcgcgctgtttccagctctgcactgtcgggcgtttcatctgcaacatggcggcaatctgcggcacggaaaacccctgccagtacagcagcgccgcctgacgacgcgggtcgtgtaaaagagtggtgtctgtggtgatggtcatgaatacctcgccgtgatgaatacacggcaaggctactgagtcgcgccccgcgattcgctaaggtgctgttgtgtcagtgataagccatccgggactgatggcggaggatgcgcatcgtcgggaaactgatgccgacatgtgactcctctaatcactattcaggactcctgacaatggcaaaaaaagtctcaaaattctttcgtatcggcgttgagggtgacacctgtgacgggcgtgtcatcagtgcgcaggatattcaggaaatggccgaaacctttgacccgcgtgtctatggttgccgcattaacctggaacatctgcgcggcatcctgcctgacggtatttttaagcgttatggcgatgtggccgaactgaaggccgaaaagattgacgatgattcggcgctgaaaggcaaatgggcgctgtttgcgaaaatcaccccgaccgatgaccttatcgcgatgaacaaggccgcgcagaaggtctacacctcaatggaaattcagccgaactttgccaacaccggcaaatgttatctggtgggtctggccgtcaccgatgacccggcaagcctcggcacggaatacctggaattctgccgcacggcaaaacacaaccccctgaaccgcttcaaattaagccctgaaaacctgatttcagtggcaacgcctgttgagctggaatttgaagacctgcctgaaaccgtgttcaccgccctgaccgaaaaggtgaagtccatttttggccgcaaacaggccagcgatgatgcccgtctgaatgacgtgcatgaagcggtgaccgctgttgctgaacatgtgcaggaaaaactgagcgccactgagcagcgcctcgctgagatggaaaccgccttttctgcacttaagcaggaggtgactgacagggcggatgaaaccagccaggcattcacccgcctgaaaaacagtctcgaccacaccgaaagtctgacccagcagcgccgcagcaaagccaccggcggtggcggtgacgccctgatgacgaactgctgaccggcgtcagtcagtccgggaaaaccttcacgattaacccttaatttcaggaaaaactatgcgccaggaaacccgctttaaatttaatgcctacctgtcccgtgttgccgaactgaacggcatcgacgccggtgatgtgtcgaaaaaattcaccgttgaaccgtcggtcacccagaccctgatgaacaccatgcaggagtcctctgactttctgacccgcatcaatattgtgccggtcagcgaaatgaaaggggaaaaaattggtatcggtgtcaccggctccatcgccagcactaccgacactgccggtggtaccgagcgtcagccgaaggacttctcgaagctggcgtcaaacaagtacgaatgcgaccagattaacttcgatttttatatccgctacaaaacgctggacctgtgggcgcgttatcaggatttccagctccgtatccgtaacgccattatcaaacgccagtcccttgatttcatcatggccggttttaacggcgtgaagcgtgccgaaacctctgaccgcagcagcaatccgatgttgcaggatgtggcggtcggctggctgcagaaataccgcaatgaagcaccggcgcgcgtgatgagcaaggtcactgacgaggaaggccgcaccacctctgaggttatccgcgtgggtaagggcggtgattatgccagccttgatgcactggtgatggatgcgaccaacaacctgattgaaccgtggtatcaggaagaccctgaccttgtggtgattgtggggcgtcagctactggcggacaagtatttccccatcgtcaacaaggagcaggacaacagcgaaatgctggccgctgacgtcatcatcagccagaaacgcatcggtaacctaccagcggtacgcgtcccgtacttcccggcggatgcgatgctcatcacgaagctggaaaacctgtccatctactacatggatgacagccatcgccgcgtgattgaggaaaacccgaaactcgaccgcgtggagaactacgagtcaatgaacattgattacgtggtggaagactacgccgccggttgtctggtggaaaaaatcaaggtcggtgacttctccacaccggctaaggcgaccgcagagccgggagcgtaaccgatgacgagtcccgcacagcgccacatgatgcgggtctcggcagcgatgaccgcgcagcgggaagccgccccgctgcgacatgcaactgtctatgagcagatgctggttaagctcgccgcagaccagcgcacactgaaagcgatttactcaaaagagctgaaggccgcaaaaaaacgcgaactgctgccgttctggttgccgtgggtgaacggcgtgctggagctgggcaaaggtgcacaggatgacattctgatgacggtcatgctgtggcgtctggataccggcgatattgccggtgcgctggagattgcccgttatgccctgaagtacggtctgaccatgccgggtaaacaccgccgtaccccgccgtacatgttcaccgaggaggtagcgcttgcggccatgcgcgctcacgctgccggtgagtctgtggatacccgcctgctgacggagacccttgaactgaccgccacggctgacatgcctgatgaagtgcgcgcaaagctgcacaaaatcaccggtctgtttctgcgtgacggtggtgatgccgccggtgcgctggcgcacctgcaacgtgcgacacagctcgactgtcaggcaggcgtcaaaaaagagattgaacgactggagcgggagctgaaaccgaagccggagccgcagcccaaagcggccacccgcgccccgcgtaagacccggagcgtgacaccggcaaaacgtggacgcccgaaaaagaaagccagttaacaaccgaatgcgccccgcgccagggcggcacgccggtcagtgacggtgaatcacctgacactgcaccggcgtccaccgcccgacttttcagaggtagtcatgatgacgctgattattccgcgaaaggaggctcccgtgtccggtgagggtacggtggtcatcccgcaaccggcaggcgacgagccggtgattaaaaacacgttcttttttcccgatatcgacccgaagcgcgtccgggaacgtatgcgccttgagcagaccgtcgcccccgcccgtctgcgtgaggccatcaagtcaggcatggctgaaacgaatgcggagctgtacgagtaccgcgaacagaaaattgccgccggttttacgcgtctggctgacgtcccggcggacgatatcgacggtgaaagcatcaaggttttttactacgagcgcgccgtgtgtgcgatggcgaccgcgtcgctttatgagcgttatcgcggtgtggatgccagtgcgaaaggcgacaagaaggctgacagcattgacagcaccattgatgagctgtggcgggatatgcgctgggcggtggcgcgcatccagggcaagccgcgctgcatcgtgagtcaaatctgatgaagacctttgcgctacagggcgacacgctcgacgccatttgtgtccgctattacgggcgcactgagggcgtggttgagaccgtgctcgccgcaaatccgggactggctgaactgggggcggtgctgccacacggcaccgccgtcgaactgcccgacgttcagaccgcgcccgtggctgaaactgtcaatctgtgggagtaacgcatgacagcagaagaaaaaagcgtcctgtcgcttttcatgattggggtgctgattgttgtcggcaaggtgcttgccggtggtgaacctatcaccccgcgtctgtttatcgggcgcatgttgctcggtggttttgtctcgatggttgccggtgttgttctggtgcagtttcctgacctgtcactgccagcggtgtgcggcatcggctccatgctgggtatcgccggttatcaggtgattgagattgccattcagcgccgctttaagggcagggggaaacagtaatgccggtaattaacacgcatcagaatatcgccgcctttctcgacatgctggccgtgtccgaagggacggcgaatcatccactgacgaaaaaccggggctatgacgtgatagtcaccggactggacgggaagccggaaattttcaccgactacagtgaccacccgttcgcacatggccgaccggcgaaggtgtttaaccgtcgcggtgaaaaatccacggcctccggtcgctatcagcagctttacctgttctggccgcattaccgcaaacagcttgccctgccggatttcagtccgttgtcacaggacagactcgccattcagttgatccgcgaacgcggagcactggatgacatccgggcgggacgcattgagcgcgccatttcacgctgtcgcaatatctgggcgtccctgccgggtgccggttacggtcagcgtgagcattcactggaaaaactggtcaccgtctggcgtaccgctggcggcgtaccggcttaaacggagtaaataccatgaagaaattatccctttcactgatgctgaacgtgtcgctggcgctgatgctggcactgtccctgatttacccgcagagcgtggccgtcaattttgtcgctgcctgggcgattctggcgacggttatctgtgtggttgccggtggtgtgggcgtgtatgccactgagtatgtgctggaacgctacgggcgggagctgccgccggaatcgctggccgtgaagattgtcacgtcgctgtttttgcagccggtgccgtggcgcagacgggcggcggctctggtagtggtggtggcgacgtttatctcgctggtcgctgccgggtggatttttaccgcgctgatttatcttgtggtgtcgctgtttttccggctgatacgtaaagcctgtcgtcagcgtcttgaggggcgggaaccatgtcaaggctgatgattgtgctggtcgtgttgttatcgctggcggtggccggtctgtttctggtgaaacacaaaaatgccagcctgcgcgcctcgctggacagggcgaacaacgtcgccagcggtcagcagacgaccatcaccatgctgaaaaatcagcttcatgttgcgctcaccagggcagataaaaacgagctggcgcaggtggcactgcgtcaggaactggagaacgccgcgaaacgtgaagcacagcgcgagaaaaccatcacgaggttacttaatgagaacgaagattttcgccgctggtacggtgctgacctgcctgatgctgtgcgccggttgcaccagcgccccgcctgcaccgacgccagtgattgtccccaacgcatgcccgaaagtgagcctttgcccgatgccgggcagtgacccgcagacgaacggcgatttaagtgccgatatccggcagcttgagaacgcgctggcacgctgtgccagccaggtaaaaatgattaaacactgtcaggacgaaaacgatgctcaaacccgacagcctgcgcagggcgctgactgatgccgtcacggtgctgaaaactaaccccgatatgctgcggatattcgtggataacgggagtattgcctccacactggcggcgtcgctgtcattcgaaaagcgttacacgctcaatgtgattgtgaccgactttaccggtgattttgacctgctcattgtgccggtgctggcgtggctgcgggaaaatcagcccgacatcatgaccaccgacgaaggccagaaaaagggcttcacgttttatgcagacatcaacaatgacagcagctttgatatcagtatcagcctgatgctgaccgagcgcacgctggtcagtgaggtggacggcgcactgcatgtgaagaatatctcggaacccccgccgccggagccggtcacccgcccgatggagctgtatatcaatggcgaactggtgagtaagtgggatgaatgagtttaagcgttttgaagaccggctgaccggactgattgaatcgctgtcaccgtcagggcgtcggcgactgagtgccgaactggcgaaacgtctgcggcagagtcagcagcgtcgggtgatggcacagaaagccccggacggcacaccctacgcgccacgccagcagcagagcgtcagaaaaaagaccggtcgcgttaagcgaaaaatgtttgcgaaacttattaccagtcgttttttgcatatccgtgccagcccggagcaggcatcaatggaattttacggcgggaagtcgccgaaaatcgccagtgtgcatcagtttggtctgtcggaagaaaaccggaaagacggtaagaaaattgattatccggcgcgtcccctgctcggctttaccggtgaggatgtgcagatgattgaagagattatcctggctcaccttgagcgttag

SEQ ID NO: 9 - DNA, 4967 bp, synthetic construct
  misc_feature 1..4967: note = Synthetic Construct
  source 1..4967: mol_type = other DNA
atgaacactctcgcaaatattcaggaactcgcgcgcgcactgcgcaacatgattcgcactggcattatcgtcgaaaccgaccttaacgccggtcgctgccgcgtgcagaccggcggcatgtgcaccgactggcttcagtggctgacccatcgcgcaggacgttcgcgcacatggtgggcaccttccgtgggggaacaggtgctgattctggccgtgggtggtgaactcgacacggcgttcgttctgccggggatttattccggcgataacccctcgccgtctgtgtcggcggatgccctgcatatccgtttccctgacggggcggtgattgaatatgaacccgaaaccagtgcactcacggtaagcggaattaaaacggccagcgtgacggcttccggttctgttactgccacggtgccggtggtcatggtgaaagcatcaacccgcgtcaccctggacaccccggaggtggtctgcaccaacaggctgattaccggcacgctggaagtgcagaaaggcgggacgatgcgcggcaacattgaacacaccggcggtgaactctcatcaaacggtaaggtactgcatacccataaacaccccggcgacagcggcggcacaaccgggagtcctttatgacagcgcgttatctcggaatgaatcgcagtgatggcctgactgtcactgaccttgagcatatcagccagagtatcggcgatatcctgcgcacaccggtcggctcacgggtgatgcgtcgtgattacggctcgttgctggcgtcaatgattgaccagccgcagaccccggcgcttgagttgcagattaaagtcgcctgttacatggcagtgctgaaatgggaaccccgcgtcaccctgtcatccgtcaccacggcgcgcagttttgacgggcgaatgacggtcacgttaaccggccagcacaacgacaccggccagccactttcattaaccatccctgtgagttgaaaccatgccgattatcgacctgaaccagctacccgcaccggatgtggtcgaggagctggactttgaaagcattctcgctgaacgcaaggcgacactgatttccctttacccggaagatcagcaggaggcggtcgcccgtaccctgacactggaatctgagcctctcgtcaaactgctggaagaaaatgcttatcgtgagcttatctggcgtcagcgtgtgaatgaggccgcacgggcggtgatgctggcctgtgccgccggtaatgaccttgatgtgattggtgccaattacaacaccacgcgcctgactatcaccccggcagatgattcgaccatcccgccgacaccggcagtgatggaatctgacaccgattatcgtctgcgtattcagcaggcttttgagggcttaagcgtcgccgggtcagtgggagcctatcagtatcatggtcgcagtgctgacgggcgtgtcgcggatatttctgtcaccagtccgtctccggcctgtgtcaccatctctgtgctgtcacgtgaaaataacggcgtcgcatccgaagacctgctggctgtggtgcgtaacgcccttaatggcgaggacgtcaggccggtggccgaccgcgtgaccgtgcagtctgccgccatcgttgaataccagataaacgccacgctttacctttaccctggtcccgaaagcgaacccatccgcgctgccgctgtgaaaaagctggaagcgtatatcacggcacagcaccggctggggcgcgacatccgtctgtctgccatttatgccgctttgcatgtggaaggtgtgcagcgtgtcgaactggctgcaccactggccgacatcgtgctcaacagtacgcaggcgtctttctgtaccgaataccgcgtcgtgaccggaggctcggatgagtgattcgcgactgctgccgaccggctcatcaccgcttgaggtcgccgccgcaaaagcctgtgcggaaattgaaaaaacgccggtcagtattcgtgaactgtggaacccggacacctgtccggcaaatctgctgccgtggctggcgtgggcgttttcggtcgacaggtgggatgaaaagtggccggaagcgacaaaacgcgccgttatccgcgatgcctatttcatccactgtcataagggcacgataggtgcaatccggcgtgtggtggagccgctcggctatctcatcaacgtgacggagtggtgggaaaacagtgacccgcccggcaccttccggcttgatattggtgtactggaaagcggtatcacagaggcaatgtatcaggaaatggaacggctgattgctgatgccaaacctgcaagccgtcaccttattggcctgaacattacccgggacattcccggctatctgttcgccggtggtgtggcttacgacggcgatgtaattacggtttaccccggataagtgaggaataatgagcataaaattcagaaccgttatcaccactgccggtgcagcaaagctggcagcggcaaccgcgccgggaaggcggaaggtcggcattaccacgatggccgtcggggatggcggtggtaaattgcctgtcccggatgccggacagaccgggcttatccatgaagtctggcgacatgcgctgaacaaaatcagccaggacaaacgaaacagtaattatattatcgccgagctggttattccgccggaggtgggcggtttctggatgcgtgagcttggcctgtacgatgatgcgggaacgttaattgccgtggcgaacatggccgaaagctataagccagcccttgccgaaggctcaggacgttggcagacctgtcgcatggtcatcatcgtcagcagtgtggcctcagtggagctgaccattgacaccacaacggtgatggcgacgcaggattacgttgatgacaaaattgcagagcacgaacagtcacgacgtcacccggacgcctcgctgacagcaaaaggttttactcagttaagcagtgcgaccaacagcacgtctgaaacactggccgcaacgccgaaagcggtaaaggccgcgtatgacctggctaacgggaaatataccgcacaggacgccaccacagcgcgaaaaggccttgtccagcttagtagcgccaccaacagcacgtctgaaacgctcgccgcaacaccaaaagccgttaagacggtaatggatgaaacgaacaaaaaagcgccattaaacagccctgcactgaccggaacgccaacgacgccaactgcgcgacagggaacgaataatactcagatcgcaaacacggctttcgttatggccgcgattgccgcccttgtagactcgtcgcctgacgcactgaatacgctgaacgagctggcggcggcgctgggcaatgacccgaattttgctaccaccatgactaatgcgcttgcgggtaagcaaccgaaagatgctaccctgacggcgctggcggggcttgctactgcggcagacaggtttccgtattttacggggaatgatgttgccagcctggcgaccctgacaaaagtcgggcgggatattctggctaaatcgaccgttgccgccgttatcgaatatctcggtttacaggaaacggtaaaccgagccgggaacgccgtgcaaaaaaatggcgataccttgtccggtggacttacttttgaaaacgactcaatccttgcctggattcgaaatactgactgggcgaagattggatttaaaaatgatgccgatggtgacactgattcatacatgtggtttgaaacgggggataacggcaatgaatatttcaaatggagaagccgccagagtaccacaacaaaagacctgatgacgttgaaatgggatgcactaaatattcttgttaatgccgtcattaatggctgttttggagttggtacgacgaatgcactaggtggtagctctattgttcttggtgataatgataccggatttaaacagaatggagacggtattcttgatgtttatgctaacagtcagcgtgtattccgttttcagaatggagtggctattgcttttaaaaatattcaggcaggtgatagtaaaaagttctcgctatccagctctaatacatccacgaagaatattacctttaatttatggggtgcttccacccgtccagtggttgcagagttaggcgatgaggccggatggcatttctatagccagcgaaatacagataactcggtaatatttgctgttaacggtcagatgcaacccagcaactggggaaattttgattcccgctatgtgaaagatgttcgcctgggtacgcgagttgttcaattgatggcgcgaggtggtcgttatgaaaaagccggacacacgattaccggattaagaatcattggtgaagtagatggcgatgatgaagccatcttcaggccgatacaaaaatacatcaatggcacatggtataacgttgcgcaggtgtaagttatgcagcatttaaagaacattaagtcaggtaatccaaaaacaaaagagcaatatcagctaacaaagaattttgatgttatctggttatggtccgaagacggaaaaaactggtatgaggaagtgaagaactttcagccagacacaataaagattgtttacgatgaaaataatattattgtcgctatcaccagagatgcttcaacgcttaatcctgaaggttttagcgttgttgaggttcctgatattacctccaaccgacgtgctgacgactcaggtaaatggatgtttaaggatggtgctgtggttaaacggatttatacggcagatgaacagcaacaacaggcagaatcacaaaaggccgcgttactttccgaagcggaaaacgttattcagccactggaacgcgctgtcaggctgaatatggcgacggatgaggaacgtgcacgactggagtcatgggaacgttacagcgttctggtcagccgtgtggatcctgcaaatcctgaatggccggaaatgccgcaataa

SEQ ID NO: 10 - DNA, 6603 bp, synthetic construct
  misc_feature 1..6603: note = Synthetic Construct
  source 1..6603: mol_type = other DNA
atgagtgactatcatcacggcgtgcaggtgctggagattaacgagggcacccgcgtcatttccaccgtatccacggccattgtcggcatggtctgcacggccagcgatgcagatgcggaaaccttccccctcaataaacctgtgctgattaccaatgtgcagagcgcaatttcaaaggccggtaaaaaaggcacgctggcggcatcgttgcaggccatcgctgaccagtcaaaaccggtcaccgttgtcatgcgcgtggaagacggcaccggtgatgacgaggaaacgaaactcgcgcagaccgtttccaatatcatcggcaccaccgatgaaaacggtcagtacaccggactaaaagccatgctggcggcggagtcggtaaccggtgttaaaccgcgtattctcggcgtgccgggactggataccaaagaggtggctgttgcactggcatcagtctgtcagaagctgcgtgctttcgggtatatcagcgcatggggctgtaaaaccatttccgaggtgaaagcctatcgtcagaatttcagccagcgtgagctgatggtcatctggccggatttcctcgcatgggatacggtcaccagtaccaccgccaccgcgtatgccaccgcccgtgcgctggggctgcgcgctaaaatcgaccaggagcagggctggcataaaacgctgtccaatgtcggggtgaacggtgttaccggcatcagcgcatctgtattctgggatttgcaggagtccggcaccgatgctgacctgcttaacgagtcaggcgtcactacgctgattcgccgcgacggtttccgcttctggggtaaccgtacctgctctgatgacccgctgttcctctttgaaaactacacccgcaccgcgcaggtcgtggccgacacgatggctgaggcgcacatgtgggcggtggacaagcccatcactgcaacgctgattcgcgacatcgttgacggcatcaatgccaaattccgtgagctgaaaacaaacggctatatcgtggatgcgacctgctggttcagcgaagaatccaacgatgcggaaaccctcaaggccggaaaactgtatatcgactacgactatacaccggtgcctcctctcgaaaacctgaccctgcgccagcgtattaccgataaatacctggcaaatctggtcacctcggttaacagcaattaaggagcctgaccgatggcaatgccgcgcaaactcaagttaatgaacgtctttctgaacggctacagctatcagggcgttgcaaagtccgtcacgctgccaaaactgacccgtaagctcgaaaactatcgcggtgcggggatgaacggcagcgcaccggtagacctcggccttgatgacgatgcgctgtcaatggagtggtcgctcggtggcttcccggattcggttatctgggagctttacgccgcaaccggtgtggatgccgtgccgattcgttttgcaggctcttaccagcgcgacgataccggcgaaacggtggccgtcgaagtggtcatgcgtggacgtcagaaagaaatcgacaccggcgagggtaaacagggagaagacactgagtcgaaaatctccgtggtctgcacctatttccggctgacgatggacggtaaggagctggtcgaaattgacaccatcaacatgattgagaaggtgaacggcgtcgatcggctggagcaacaccgccgcaatatcggcctgtgattttcatccggtcagcctggctggccggttaaccctgattcagaagtgagaaaaccatgaacaaagaaaatgtcattaccctggacaatccggtcaaacgtggtgagcaggttatcgaacaggtcacgctgatgaaacccagtgccgggacgctacgcggtgtcagtctggctgcggttgcaaactccgaagtcgatgcactgattaaggtgctgccgcgcatgacggcaccgatgctgaccgagcaggaagtcgccgcgctggaactgcctgaccttgtggcgctggccggtaaggtggtcggttttttgtcgccgaactcggtgcagtgacgtttccgaaaaatctctcggtcgatgacctgatggcggatgtggcagtgatatttcactggccgccatcagaactgtatcccatgagcctgaccgaactcatcacatggcgcgaaaaggcgctccggcgaagcggaaacacgaatgagtaacaatgtaaaattacaggtattgctcagggctgttgaccaggcatcccgcccgtttaaatccatccgcacagcgagcaagtcgctgtcgggggatatccgggaaacacaaaaatcactgcgcgagctgaacggtcacgcatcccgtattgagggattccgcaagaccagtgcacagctcgccgtgactggtcatgcacttgaaaaggcacggcaggaggccgaagcccttgccacacagtttaaaaacaccgaacgtccgacccgtgctcaggcgaaagtcctggaatccgcaaagcgtgcggcggaggacttacaggcgaaatataaccgcctgacagattccgttaaacgccagcagcgggaactggccgctgtgggaattaatacccgcaatcttgcacatgatgagcagggactgaaaaaccgtatcagtgaaaccaccgcacagcttaaccgtcagcgtgatgcgctggtgcgtgtcagtgcgcaacaggcaaaacttaacgcagtaaaacagcgttatcaggccggaaaggaactggccggaaatatggcctcagtgggcgctgccggtgtggggattgcggcggcgggaacgatggccggtgttaagctactgatgcccggttatgagtttgcgcagaaaaactcagaattacaggctgtgatcggagtggcaaaagactccgccgaaatggccgcactccgcaagcaggcgcgccagctcggcgacaataccgccgcctcggcagatgatgcagccggtgcgcagattattattgcgaaagccggtggggatgttgatgccattcaggcggcaacgccggtcacgctgaacatggcgctggcgaaccgtcgcacaatggaagaaaacgccgccctgctgatggggatgaaatccgcctttcagctttcaaacgataaggtcgctcatatcggggatgttctctccatgacgatgaacaaaaccgccgccgattttgacggcatgagcgatgcgctgacctatgccgcacctgtggcaaaaaatgccggtgtcagcattgaagaaaccgccgcaatggtcggggcgctgcatgatgcaaaaatcacaggctcaatggcggggacgggaagccgtgccgtgttaagccgcctgcaggcaccgacgggaaaagcatgggatgcactcaaagagcttggagtgaaaacctcagacagcaaaggaaacacccggccaatatttaccattctgaaagaaatgcaggccagttttgagaaaaaccggctcggtactgcccagcaggctgaatacatgaaaactattttcggggaggaggccagctcagccgctgccgtgctgatgactgccgcctcaaccggaaagctggacaaactgaccgctgcgtttaaagcctcagacgggaagaccgccgagctggtaaatatcatgcaggacaacctaggcggtgactttaaagcgtttcagtccgcttatgaggcggtggggactgacctgtttgaccagcaggaaggcgcgctgcgtaagctcacgcagacggccacaaagtatgtgttaaaactcgacggctggatacagaaaaacaaatcactggcgtcaaccatcggcatcattgccggcggtgcactggcgcttactggcatcatcggtgccattggcctcgtagcctggccggttatcaccggcatcaatgccatcatcgcggcagcaggcgcaatgggggcagtcttcacgacggttggcagtgctgttatgaccgccatcggggctattagctggccggttgtggccgtggtggctgccattgtcgccggtgcgttgcttatccgtaaatactgggagcctgtcagcgcattctttggtggtgtggttgaagggctgaaagcggcatttgcgccggtgggggaactgttcacgccacttaaaccggtttttgactggctgggcgaaaagttacaggccgcgtggcagtggtttaaaaacctgattgccccggtcaaagccacccaggacaccctgaaccgttgccgtgacacgggcgtcatgttcgggcaggcactggctgacgcgttgatgctgccgcttaatgcgttcaacaaactgcgcagtggtattgactgggtactggaaaaactcggtgttatcaacaaagagtcagacacacttgaccagaccgccgccagaactcataccgccacgtatggtaccggtgactatattccggcgaccagctcttatgcaggctatcaggcttatcagccggtcacggcaccggctggccgctcttatgtagaccagagtaaaaacgaatatcacatcagcctgacgggggggactgcgccggggacacagcttgaccgccagttacaggatgcgctcgaaaaatacgagcgggataaacgtgcgcgcgcccgtgccagcatgatgcatgacggttaaggaggtgacgaaaaatgatgctcgcgttaggtatgtttgtttttatgcgccagacgctgccacaccagaccatgcagcgtgaatcagattatcgctggccgtcaaattcccgtatcggtaaacgggatgcctttcagtttctcggtgtgggtgaggaaaacatcacgctggccggtgtgctttatcccgaactgaccggcggcaagctgacgatgaccacgctcaggctgatggcagaggaggggcgggcgtggccgttgctggatggcaccggcatgatttacggcatgtatgtcatcagcagggtgagtgaaacagggagtattttctttgcagacggcacaccccggaaaattgattttacgctgtcactcacccgcgttgatgaatcactggccgcgctttatggcgatatcggtaaacaggcggaatcgctcatcggtaaggccggcagtatggcgaccagattcacaggtatgacgggggcgggataatgctggatgcgctgacatttgatgcaggcagtacgctgacgccggattacatgctgatgctcgacagcagggatattaccggcaatatcagcgaccgtctgatgagcatgaccctgacggataaccggggctttgaggctgaccagcttgatattgaactgaacgatgccgacgggcaggtcgggctgccggttcgtggcgctgtcctgacggtgtatatcggctggaaaggttttgccctggtatgcaaagggaaatttaccgttgatgaggttgaacaccggggcgcaccggatgtagtcaccatccgcgcccggagtgcagattttcgcgggacgctcaattcccgccgggaaggctcctggcatgacaccacgctcggtgcgattgttaaggcgatagccacccgtaacaggctggaagccagtgtcgctccgtcactggccggaataaaaattccacacatcgaccagtcgcaggagtctgatgcgaaattcctgacccgtcttgcagaacgcaacggcggtgaggtgtcggtaaaaatgggaaaactgttgtttctcaaagcggggcagggagtgacggccagcggtaaaaaaatcccgcaggtcaccataacccgcagcgacggcgaccgccatcattttgcgattgctgaccgtggagcctacaccggtgtaacggcaaaatggctacacactaaagacccgaagccgcaaaagcagaaggtaaaactgaaacgcaaaaagaaagagaaacacctgcgcgcactggagcacccgaaagcgaaaccggtcaggcagaagaaagcgcctaaagtaccggaagcgcgtgaaggtgaatacatggccggtgaggctgacaacgtttttgccctgaccacggtatatgccacgaaagcgcaggccatgcgcgccgctcaggcgaagtgggataaactgcaacggggcgttgcggagttctctatcagcctggctaccggtcgggcagatatttacacggaaacaccggtcaaagtgtctggctttaagcgcgtcatagacgagcaggactggacaatcactaaggtgacacattttctgaataatagcggcttcacgacgtccttagagcttgaggtcaggctttctgatgtggagtacgaaacagaagatgatgagtgatgtttttgttttatctgtttgttttgtaaggataaattaactaaaatggcaccatcaacaaaaccggaagaggtgctcgcgatgtttcattgtcctttatgccagcatgccgcacatgcgcgtacaagtcgctatatcactgacacgacaaaagagcgttatcatcagtgccagaacgtgaattgcagcgccacgttcatcacttatgagtcggtacagcgatacatcgtgaagccgggagaagtccacgccgtaaggccgcacccgttgccatcagggcagcaaattatgtggatgtaa

SEQ ID NO: 11 - DNA, 12 bp, synthetic construct
  misc_feature 1..12: note = Synthetic Construct
  misc_difference 2: note = n = A,T,C or G
  misc_difference 9: note = n = A,T,C or G
  source 1..12: mol_type = other DNA
angatttantcc

SEQ ID NO: 12 - protein, 192 aa, synthetic construct
  REGION 1..192: note = Synthetic Construct
MKTESYFKEYNQFVLDQHKAIQELEQERNALESKIKLDKSTYKQLIMDGQDDKADNLYQATDADEKKLKALNKRLETKKSVSKEVKYQKTIELLKHQSELSSLYESEKQSAIEKLKKAVDAYNEIIDEIEDINDRYEDEHQQYASVYSQEQLYDDKEARKALNGHFKENIFTSFINGNDLPYEHNNKLFLKC

SEQ ID NO: 13 - protein, 72 aa, synthetic construct
  REGION 1..72: note = Synthetic Construct
MKTKYELNNTKKVANAFCLNEEDTNLLINAVDLDIKNNMQEISSELQQAEQSKQKQYGTTLQNLAKQNRIIK

SEQ ID NO: 14 - protein, 113 aa, synthetic construct
  REGION 1..113: note = Synthetic Construct
MDKQQIKDFVCDYHERTRSDVLIDDDINTDEFFSIADENSNEWMADDNIDDHIVKNHLEMIVDRVANDKEFYIFDSLIQGRSYQDISGVLDCSEQSVRFWYETLLDKIVEVIE

SEQ ID NO: 15 - protein, 113 aa, synthetic construct
  REGION 1..113: note = Synthetic Construct
MESIAEKETYHLPTEHLQVFNVIKNTSNKYITKTKILNQLGYEYNSSNERWLRRVINSLVYDYGYPIGCSYKPSERGYYIITTEQEKQQAMRSIKKLADGSMKRYEALKRIEV

SEQ ID NO: 16 - protein, 191 aa, synthetic construct
  REGION 1..191: note = Synthetic Construct
MIAYPIRVGSVYRGEQMKLLKTKNCLYYRNGDNKLSEYQLLTQFNPTFINKKIRMCEFQIESMYHMSASTTTCDEMMGVVSVSYPIEKLVIKIIETKARLQNYKNRSISNMVLLKTVLNHYTEKEQKKVVKYMRSNGRYKPYNVIERLQVDLYQASIKQRSERQKQRNIAIENSKIARVNAYHQSSYVKVV

SEQ ID NO: 17 - DNA, 27079 bp, synthetic construct
  misc_feature 1..27079: note = Synthetic Construct
  source 1..27079: mol_type = other DNA
atgaacgaaaaacaaaagagattcgcagatgaatatataatgaatggatgtaatggtaaaaaagcagcaattacagcaggttatagtaagaaaacagcagagtctttagcaagtcgattgttaagaaatgttaatgtttcggaatatattaaagaacgattagaacagatacaagaagagcgtttaatgagtattacagaagctttagcgttatctgcttctattgctagaggagaacctcaagaggcttacagtaagaaatatgaccatttaaacgatgaagtggaaaaagaggttacttacacaatcacaccaacttttgaagagcgtcagagatctattgaccacatactaaaagtacatggtgcgtatatcgataaaaaagaaattactcagaagaatattgagattaatattggtgagtacgatgacgaaagttaaattaaactttaacaaaccgtctaatgttttcaatagaaacatattcgaaatactaaccaattacgataacttcactgaagtacattacggtggaggttcgagcggtaagtctcacggcgttatacaaaaagttgtactcaaagcattgcaagattggaaatatcctaggcgtatactgtggcttagaaaagtacaatcaacaattaaagatagtttgttcgaagatgttaaagattgtttgataaactttggtatttgggacatgtgcctttggaataagactgataacaaagttgaattgccaaacggcgcagtttttttgtttaaaggattagataacccagagaaaataaagtcgataaaaggcatatcagacatagtcatggaagaagcgtctgaattcacactaaatgattacacgcaattaacgttgcgtttgagggagcgtaaacacgtgaataagcaaatatttttgatgtttaacccagtatctaaactgaattgggtttataagtatttctttgaacatggtgaaccaatggaaaatgtcatgattagacaatctagttatcgagataataagtttcttgatgaaatgacacgacaaaacttagagttgttagcaaatcgtaatccagcatattacaaaatttatgcgttaggtgaatttgctacactagacaaattggttttccctaagtatgaaaaacgtttaataaataaagatgagttaagacatttaccttcttattttggattggactttggctacgttaatgatcctagtgcttttatacattctaaaatagatgtaaagaaaaagaagttatacatcattgaagagtatgttaaacaaggtatgctgaatgatgaaatagctaatgtcataaagcaacttggttatgctaaagaagaaattacagcagatagtgcagaacaaaaaagtatagctgaattaaggaatctagggcttaaaaggattttaccaaccaaaaaagggaagggctcggttgtacaagggttacaattcttaatgcaatttgaaatcattgttgatgaacgttgtttcaagactattgaagagtttgacaactacacatggcaaaaggacaaagatacaggtgaatataccaatgaaccagtagatacatacaatcattgtatcgattcgttgcgttattcagtggaacgattctacagaccggttagaaaacgcacaaatctcagttcgaaagttgacacaataaaatctctaggattataggagggaacaaatgttaaaagtaaacgaatttgaaacagatacagatctacggggaaacataaattacttatttaatgatgaagccaatgttgtttacacatatgacgggacggaatccgatttattacaaaacgttaatgaagtaagtaaatacattgaacatcacatggattaccaacgacctagattgaaagtgttaagtgattattacgaaggtaaaactaagaacttagttgagttaacacgacgcaaagaagagtacatggcagataaccgtgtagcgcatgattacgcatcttatattagcgattttatcaacggctatttcttgggtaatccaattcaatatcaagatgatgacaaagatgtattagaagttattgaggcgttcaatgatttaaatgatgttgagtcacacaatagatctttaggattagatttgtcaatttatggcaaagcttatgagttaatgattagaaaccaagatgatgaaacgcgtttatacaagagtgatgcaatgagtacttttgtcatatacgacaatacaattgaacgtaatagtatcgcaggcgttagatatttaagaactaaaccaatagacaagactgacgaagatgaagtgtttacagttgatttattcacttcacacggtgtttatagatatcttaccagtagaacaaatggattgaagctcacaccacgtgaaaacggttttgaatcacactctttcgaacgtatgcctattacagaatttagcaacaacgaaagaagaaaaggggattatgagaaagtaatcactttaattgatttgtatgataatgctgaatcagatactgctaactatatgagtgatttaaatgacgctatgttacttattaaaggtaatttaaatttagatcctgtagaagttagaaaacaaaaggaagctaacgtgttgtttttagaaccgactgtttatgctgatagcgaaggtagagaaacagaaggctctgttgatggtggttatatttataagcaatacgatgtacaaggtaccgaagcttataaagaccgtttaaacagtgatatacacatgtttaccaacacgcctaacatgaaagatgataactttagcggcactcaatcgggcgaggcaatgaaatacaaattatttggattggaacaacgtactaaaactaaagaaggattgtttactaaagggttaagacgtcgtgctaagttgttagagacaatacttaaaaatacatggtcgattgacgctaacaaagatttcaatactgttagatacgtatacaacagaaacttacctaaatcattgattgaagaattaaaagcttatattgattctggtgggaagattagccaaacaactttaatgtctctattctcgttcttccaagaccctgaattagaagttaagaaaatcgaagaagatgagaaagaatctattaaaaaagctcaaaaaggtatttataaagaccctagagacatcaatgatgacgaacaagatgatgatacaaaagatactgttgataaaaaggaatgattgtaattgcctaacaaaaacactcaagaatattgggaagaacgcggacgcaaagcaatcgagaatgagttgaagcgtgataaaactaaagctgaagaaatagaacgtatattgaatatgatgattaagcgcattgaaaaagagatcaatgcgtttattgtcaagtacggagattttgcaggcgttacattacaagaagcacaaaagattattgatgagttcgatgtaaaagcgtttcaagaagaagcaaaaagattggtcgaaaacaaggagtttagcgatagagcaaatgaagaattaaagaagtataacacgaaaatgtatgtatctagagaacagatgttaaagattcaaatagaattcttaattgcttatgcaacagctcaaacagaattatcgatgagggaatatttcgaatcaacagcttatcgtgtgttcagtgatcaagcgggtattttaggtgaaggtgtacaagtagctaaagaagttatagatacaatcgttgatacacaatttcatggtgtcgtttggtcagagcgattatggactaataccgaagcaatgaaacaagaagtagaagaaataattgctaatgtagttattagaggtcgacatcctaatgaatatgttaaagatatgcgcaagcacttaaataaattcgaaggcacagcacgacaaaagaccgcagcaattaaatcattgctttatacggaatcggcacgtgttcacgcacaatcaagcattgacagcatgaaagaaatttcaccggaaggatattatatgtatattgcaaaaatcgataatagaacaactaaagtatgcaaagggcttaatggagaaatattcaaagttaaagacgctaaaattggtgttaatttctatcctatgcatatcaattgtcgttcagattgcgctttactacctaaatctatgtggccgaaaaaaccaagcaagaaacgaaaaacaaaatacttcggagggaaagtgaaaagcggtgattgatttaaaagtgaagttttttaaaggcaagttagttttgtatgacagtaaattaaatgtttggaggatactaatatgagtaatactgacaaataccttagagacatagcaagagaattaaaaggtatacgtaaagagttacaaaagcgaaacgaaacagttattattgatgcaaacttagacagtttaaggtcggcagtattagccgataaagaaaaatcgaaatataatgaacctctcttttaatagctagcacttaattgtgttggctattttttatgtccaaaacgtgctgatgacataaaaagcacgcatggaaaaacagtcgacagactataaatggaggtatatctcatggaagaaaataaacttaagtttaatttgcaattttttgcagaccaatcagatgatccggacgaaccaggcggagatggtaaaaaaggaaatcctgataagaaagaaaatgacgaaggtactgaaataactttcacgccagagcaacaaaagaaagttgatgaaatacttgaacgtcgtgtagcccacgaaaagaaaaaagctgatgagtatgcaaaagaaaaagcagcagaagctgctaaagaagctgctaaattagcgaaaatgaacaaggatcaaaaagatgaatatgaacgcgaacaaatggaaaaagaactggaacaattacgttcagaaaaacaattaaacgaaatgcgttcagaagcacgaaaaatgttgagtgaagcggaagttgattcatcagatgaggttgtcaatttagttgtaacagatactgctgaacaaactaaattgaatgttgaagctttttctaatgcagtaaaaaaagcggttaatgaagcggttaaggttaacgctagacaatcgccattgactggtggagattcatttaatcactcgactaaaaataaaccgcaaaacttagctgaaatagctagacaaaaaagaattattaaaaattaacggaggcatttaaatggaacaaacacaaaaattaaaattaaatttgcaacattttgcaagtaacaatgttaaaccacaagtatttaaccctgacaatgtaatgatgcatgaaaagaaagatggcacgttgttaaacgactttacaacacctatcttacaagaggttatggaaaactctaaaatcatgcaattaggtaagtacgaaccaatggaaggtactgagaagaagtttactttttgggctgataaaccaggtgcttactgggtaggtgaaggtcaaaaaatcgaaacgtctaaggctacttgggttaatgctacaatgagagcgtttaaattaggggttatcttaccagtaacaaaagaattcttgaattacacttattcacaattctttgaagaaatgaaacctatgattgctgaagctttctataaaaagtttgacgaggcaggtattttgaatcaaggtaacaatccgttcggtaaatcaattgcacaatcaattgaaaaaactaataaggttattaaaggtgacttcacacaagataacattattgatttagaggcattgcttgaagatgacgaattagaagcaaatgcatttatctcaaaaacacaaaacagaagcttgttacgtaaaattgtagatcctgaaacgaaagaacgtatttatgaccgtaacagtgattcgttagacggtctacctgtggttaaccttaaatcaagcaacttaaaacgtggtgaattaatcactggtgacttcgacaaattgatttatggtatccctcaattaatcgaatacaaaatcgatgaaactgcacaattatctacagttaaaaacgaagatggcacacctgtaaacttgtttgaacaagacatggtggcattacgtgcaactatgcatgtagcattgcatattgctgatgataaagcgtttgctaagttagttcctgctgacaaaagaacagattcagttccaggagaagtttaataaataattaggagtggtaacatgcccgaaatcattggaattgttaaagtagattttacagatttagaagataacagacatgtctatatgaaagggcatgtctaccctcgtaaaggttataatcctacagatgaacgtatcaaagctttagctagtgttgaaaataaacgcaacaaacaaatgatttacattgtaaatgacaaattaaccaaaaaagaacttgtcgaaatagcaagtgttgctggcttacaagttgatgaaaaacaaacaaaagctgaaattatcaatgcttttgagtcactagagtaggtggttatatgactacgctagctgatgtaaaaaaacgtattggtcttaaagatgaaaagcaagatgaacaattagaagaaatcataaaaagttgtgaaagccagttgttatcaatgttacctattgaagttgaacaaataccggaaaggtttagttacatgattaaagaagttgcagttaaacgctacaacaggattggtgctgaaggtatgacatcagaagcggttgacggacgtagcaatgcgtatgaattgaacgatttcaaggagtatgaagctattattgataattactttaatgctagaacgagaactaaaaaaggaagggctgtgttcttttgagatatgaagatagagttatttttcaattagaacaagtagcaacttacaatcctaaaactagcaaaaaagaaaacacactaatcacttatgatgcgataccatgcaatattaaccccatttctagagcaagaaagcaacttgaatttggtgatgtaaaaaacgatgtaagtgttctgaggataaaagaatcaatatcttaccctgttagccacgtgttggttaatggcattcgctacaagatagttgatacaaggatatacagacacgaaacgtcatattatatcgaagaggtcaattgatgaatatagatggattagacgcactgttaaaccaatttcacgatatgaaaaccaacattgatgatgatgtagatgatattttacaggaaaacgccaaagaatatgtagtacgagctaaattgaaagctagagaagtaatgaataagggttattggactggtaatttatcacgcaatatcagatataaaaaaactggcgatttgcaatacactatcacatcgcacgcagcttatagtggtttcttagaatttggtactcgatacatggaggctgaaccttttatgtggccggtatacgaagtgattaggaaatcaactgtagaagaattgaaagcgttgtttgaataggagataaaagcatgacaccgaacttacaactttataataaagcgtatgaaacgctacaaggatatggattccctgttatttctcgtaaagagatgcaacaagagattccgtatcctttttttgtaataaaaatgccggagtcaaatagaagtaagtacacgtttgatagttattctggcgatacgaatttagttattgatatttggagtgtaagcgatgatttaggacatcatgacggacttgttaaaagatgtattgatgatttaacacctagcgttaaaacaaacgattatgactttgaagaagaagatactaacatcacacagttagttgatgatactaccaatcaagaattgctacacacatcagtaacgatatcttacaaaacattttaaaaaacggaggaatattgaatggcaaatatgaaaaatagtaatgatcgtattattttatttagaaaagctggcgaaaaagtagatgctactaaaatgctttttttaactgaatacggcttatcacatgaagctgatacagatacagaggatacaatggacggttcttataacactggtggttctgttgagtcaacaatgtctggtactgctaaaatgttttatggtgacgattttgcagatgaaattgaagatgcagttgtagatcgcgtattgtatgaagcttgggaagttgaaagtagaataccaggcaaaaatggggattccgctaaatttaaagcgaaatatttccaaggtttccacaataaatttgaattaaaagcagaagctaacggtattgatgaatatgaatatgaatatggagtgaatggtcgtttccaacgtggatttgcaacactacctgaggctgtaacaaagaaacttaaggcgactggatacagattccacgacactacaaaagcagatgcattaactggcgaagatttaacagcaattccacaacctaaagtagattcaccaccggttgcaccaagagaggtataaaaatagggcgttaagccctttttattttgtttaaattaattatgaatggagattttaagttatgaatgtagaaattaacggaaagtcattagaattaagttttggttttaaatttttaagagaaatcgataaccgattaggtttaaaagttgaacaagcttctatcggtcaaggtgtatcaatgttgcctgtaggtttagaaagtggaaatccggttgtgattggcgaagttttaatcgcagctacatctcacttaaaaaaacaagcaattactattaataacattgatgaagcattagatgaaatcgcagaaaatatcggactagaagaattcggttcggatattttaacggagttgggaaagcgacctatgacccgaaacctagtcgaagtagtggaaacggaagaaaaaccagcggaagcctaataacttacgacagaatcgttataacttgtatgtcaacacttggtattacagatttgaacgttattgagcaaatgacattaacagaatataactatcgaatgtatgcgaaagagtatgaaatgctaacccaagaattcgaacgttacaaacttgcgtttgctattcgtgatgctgcagctactaaaaatgttgggacagaaaataaacctaaagaggaatatgtttttaacaatgcaaacgacgtattgccttatgaagaaaatatccaacggcttaacgaaggtaaagatataagatttagtagcgaacgtgatgaatacgaaccacaaaataatgaattctttaaagttatagcagaatttaataagcaatagaaagagaggtgttaatgtgacggaatataaaattaaagcgactattgaagctagtgtagccaaattcaaaaggcaaattgatagtgcggttaagtctgtgcaaagatttaaacgagtagcagatcaaactaaagatgttgaattaaacgctaacgataaaaatttacaaaaaactatcaaagttgctaaaaagtctttagatgcctttagtaacaaaaatgtaaaagctaaattagatgctagtatacaagatttacaacaaaaggtactagaatcgaattttgaactagacaaactaaactctaaagaagttacaccagaagttaagttgcaaaaacaaaagttgattaaagatatcgctgaaacagaagctaaattatcagaattagaaaagaaacgtgtcaatattgacgtcaatgcagataacagtaaattcaatcgagtgttaaaagtatctaaagctagtctcgaagcattaaataggtctaaagccaaagctattatagacgtggacaatggtgttgctaactctaaaatcaaacgtactaaagaagaacttaaaagtattccgaacaaaactagatctcgacttgatgtagatacagggctttctataccaactatttatgcgtttaaaaaatcattagacgcattgccgaacaaaaaaacaacaaaggtagatgtcgatactaatggtttaaagaaagcttatgcctacataataaaagcaaatgacaattttcaaagacagatggggaatttagctaatatgttccgtgtgttcggtactgtaggttctaatatggttggtggattacttacatcatcttttagtatcttaatacctgtaatagcgagcgtagtacctgtagtatttgcgctattaaacgctatcaaagtgttaactggtggtgtacttgctttaggtggtgccgtagcaatagcgggagcaggatttgtagcgtttggcgcaatggctatcagcgctataaagatgcttaatgatggcactttacaagctagctcagcaacaaacgaatacaaaaaagcgttagatggcgtaaagtcagcatggactgatattataaagcaaaatcaatccgctatcttcacaactcttgcaaatggtttaaatactgttaaaactgcaatgcagagcttacaaccgttttttagtggtatttcaagaggaatggaagaagcgtctcaaagcgtgcttaaatgggctgaaaatagcagtgtagcttcaagattctttaatatgatgaatacaacgggtgtttcggtatttaacaagctattaagtgctgcaggtggttttggtgacggattagtcaatgtattcacgcaattagcaccactgtttcaatggtcggctgattggttggatagattaggtcaatctttctctaactgggctaatagtgcagctggagaaaattcgataactcgttttattgaatacacaaaaacaaacttacctatcattggtaatattttcaaaaatgttttcgttggaattaacaatttgatgaatgcattcagcggatcatcaactggcatattccaatctcttgaacaaatgacagctaagtttagggaatggtctgaacaagtaggacaatctcaagggtttaaagactttgtcagttatatacaaacaaatggaccactaataatgcaattgattggaaacatcgcaagaggattagttgcattcgcaacagcaatggctcctatagctagtgcagtattacgcgttgcagttgcaataactggttggatagctaacttgtttgaggcgcatccagctacagcacaattagttggtgtcattataactttagttggtgcatttagatttttaataccgattattcttgctgtatctaactttatgggtggcggattaataggtagaatcattgcattagtaagtaagttcggtttattaagagcgggattaacaattttaaaaggtgcgttcatgttattaaaaggaccattaaaaattatatcagttatattccaattgttattcggtaagattggattaattagaaatgctatcacaggactagtaactgtgtttggtattttaggcggtccaataacaatagtaattggtgtaattgctgcattaatagctatattcgttttattgtggaataaaaatgaaggattcagaaactttattataaatgcttggaatgcgataaaaacgtttatggttaatgtttggaatgtattaaaagctgtagcttcggttgtatggaatgctattttaacagctatcactacagcagtatcgaatgtttacaattttataatgattgtttggaatcaaatagtcgcttatttacaagggctatggaatggaattatcgctattgcaacaacagtatggaaccttttagttacaatcattacaactgttttcacgacgataatgacaatagttatgacgatatggacagctatttggacgttcttaagtacaatctggaatacgataattacaatcgctactacgatttggaatttgttagtcactgtaataactacagtgtttaccacaattatgactatcgcaataacaatttggaacgctatttggacgttcttacaaacgttgtggaacactatagttactgtggcaactaaggtttggaacgctatcactacagctatatctactgcgttacaagcggcatggagttttatttctaatatatggaatacgatttggagtttcttatctggtatattaacgacaatttggaataaagttgtaagcatattcacacaagttgtttcaactatatcagacaaaatgtctcaagcttggaacttcattgtcactaaaggtatgcaatgggtatctactataacaagtacgctaattaactttgttaatagagttgttcaaggattcgttaatgttgtaaacaaagttagtcaaggtatgacaaatgcagtaaataaagttaaaagctttgtggatgactttgtatcagcaggtgctgatatgatccgtggtttgatgagaggtattggtaatatggctagagacttagctgaaaaagcagctagtgtagcaaaaggtgctttaaatgcagccaaaagagcgctaggtattcactcaccttcacgtgaattcatggatgttggtatgtattcaatgttaggtttcgttaaaggtatagataatcattcaagtaaagttatccgtaatgtttctaatgttgcagataaagtagttgatgcatttcaacctacattaaacgcacctgacatttctagtattacaggaaacttaagtaatttaggtggaaatataaatgcgcaagtacaacacacacattctattgaaacatcaccgaacatgaaaactgttaaagttgaattcgatgtcaataacgatgcgcttactagtattgttaacggcagaaatgctaaacgcaattctgagtattacttataaaggaggttacaaatggacatagaattaacaaaaaaagatggtactgtaatcaaattaagtgaatacgggtttatcgttaacgatatagtaattgatagcatgcaaatcaacacaaagtatcaagacaaagaaaatatgaacggtcgtatattaatggggagcaattatatcagtagagatatagttgttccttgtttttgtaaagttaaaaatcgttcagacattgcttatatgcgagatatgttgtattcgttaacgacagacatagaacctatgtatttgcgagaaatcagaagaaaagaagagttgaattacaggtttactcaaccaacttctgatgattacgtgaaattagataaaaacaacttcccggattacgaatattcaagacacgatcaacaaatttatgtaaatggtaaacagtataaagttatttttaacggagttataaaccctaaacaaaaaggtaataaagtttcttttgaactaaaattcgaaactacagaattaccatacggtgaaagtattggaacaagcctagagttagaagaaaacaaaaaggttggattgtggtcgtttgattttaatattgattggcatgcaggcggagacaaaagaaagtatacatttgaaaatttgagcaaaggtacagtttactatcatggtagtgctcctaacgaccaattcaacatgtataaaaagataacaattattttaggcgaagatacagaatcgtttgtatggaatttaacgcatgctgaaataatgaaaatcgaagggatcaaactaaaagctggagacagaattgtttatgatagcttccgagtttataaaaacggtgttgaaataagtaccgaaacgaatatagcccaaccaaaatttaaatacggagctaataaatttgagtttaatcaaacggtacaaaaagttcagtttgatttgaaattttattataagtaggtgtcagaatgacaataactattaaaccacctaaaggtaatggcgcacctgtaccagtagaaacaactttagtaaaaaaagttaatgctgacggtgtattaacttttgatattctagaaaataaatatacttatgaagttattaacgctatagggaaaagatggattgttagtcatgtcgaaggtgaaaacgacaagaaagaatatgtaataactgtcattgataggaaatcagaaggcgacagacaactggttgaatgtactgctagagagattcccatagacaagttaatgattgatagaatttatgttaatgtaacaggatcttttacagtagaaagatattttaacattgtgtttcaaggtactggaatgctttttgaagtcgagggcaaagttaaatcttcaaagtttgaaaatggtggtgaaggcgatacaaggttagaaatgtttaaaaagggattagaacatttcggtttagaatataaaataacgtatgacaaaaagaaagacagatataagtttgtattgacgccttttgcaaatcaaaaagcgtcttattttatttctgacgaagtcaacgccaacgctataaaactcgaggaagatgcaagtgatttcgccaccttcattagaggatatggtaattattcaggagaagaaacattcgaacacgctgggctcgtaatggaagctagaagtgcattagctgaaatatacggcgacatccacgcagaaccatttaaagatggtaaagtgactgaccaagaaactatggataaagaattacaatcgagattgaaaaagtcgttaaaacaatctttgtctttggactttttggtgttaagagaatcatatccagaagcagacccacaacccggagacatagtacaaataaaatctaccaaactaggtttgaatgatttagtccgtatagtacaagttaaaacgattaggggtataaacaatgtaattgttaagcaagatgtaacgcttggtgagtttaatcgagaacaacgatatatgaaaaaagttaatactgcagctaactatgtttctggattaaatgatgttaacctttctaatcctagtaaagcggcagaaaacttgaagtctaaagtagcgtcaatagctaaatcaacactcgatttgatgagtagaactgatttgattgaagataaacaacagaaggtaagctctaaaactgtgactacatctgacggcactatcgttcatgattttatagataaatcaaacattaaagatgtaaaaacgattggaacgattggcgattctgtagctagaggatcacatgcgaaaactaatttcacagaaatgttaggcaagaagttaaaagctaaaacgaccaaccttgcaagaggtggcgcaacaatggcaacagttccaataggtaaagaagcggtagaaaacagcatttatagacaagcagagcaaataagaggagacctaatcatattacaaggtacagatgatgactggttacatggttattgggcaggcgtaccgataggcactgataaaaccgacactaaaacgttttacggcgccttttgttctgcaattgaagttatcaggaaaaataatccagcttcaaaaatacttgtaatgacagctactaggcaatgccctatgagtggtacaacgatacgccgtaaagatacggacaaaaacaaactagggttaactttagaggattatgtcaatgctcagatattggcttgtagtgaattggatgtaccagtatatgatgcttatcacacagattatttcaaaccatataatccagcatttagaaaatctagcatgcctgatggattacatcctaatgaaagaggtcatgaagttattatgtatgagcttattaaaaattattatcagttttatggatagtaaaggaggaaaacatgagtaataaactaattacagatttaagtagagtctttgactacagatatgtagatgaaaatgagtataactttaaacttatttcagacatgctgacggattttaatttctctcttgaataccacagaaataaagaggtattcgcacatgatggagaacaaataaagtatgaacatttaaatgttacaagtaacgtctctgactttttaacatatttaaacggtcgatttagcaacatggtactaggtcataacggcgacggtatcaacgaagtaaaagacgcgcgcgttgataatacaggttatggtcataagacattgcaagatcgtttgtatcatgattattcaacactagatgttttcactaaaaaggttgagaaagctgtagatgaacactataaagaatatcgagcgacagaataccgattcgaaccaaaagagcaagaaccggaatttatcactgatttatcgccatatacaaatgcagtaatgcaatcattttgggtagaccctagaacgaaaattatttatatgacgcaagctcgtccaggtaatcattacatgttatctagattgaagcccaacggacaatttattgatagattgcttgttaaaaacggcggtcacggtacacacaatgcgtatagatacattgatggagaattatggatttattcagctgtattggacagtaacaaaaacaacaagtttgtacgtttccaatatagaactggagaaataacttatggtaatgaaatgcaagatgtcatgccgaatatatttaacgacagatatacgtcagcgatttataatccggtagaaaatttaatgatttttagacgtgaatataaacccactgaaagacaacttaagaattcgttgaactttgttgaggttagaagtgctgacgatattgataaaggtatagacaaagtattgtatcaaatggatatacctatggaatacacttcagatacacaacctatgcaaggtatcacttatgatgcaggtatcttatattggtatacaggtgattcgaatacagccaaccctaactacttacaaggcttcgatatcaaaacgaaagaattgttatttaaacgtcgcatcgatataggcggtgtgaataacaactttaaaggagatttccaagaggctgagggtctagatatgtattacgatctagaaacaggacgtaaagcacttctaatcggggtaactattggacctggtaacaacagacatcattcaatttattctatcggtcaaagaggtgtaaaccaattcttgaaaaacatcgcacctcaagtatcaatgactgattcaggcggacgtgttaaaccgttaccaatacagaacccagcatatctaagtgatattacggaagttggtcattactatatctatacgcaagacacacaaaatgcattagatttcccgttaccgaaagcgtttagagatgcagggtggttcttggatgtactgcctggacactataatggtgctctaagacaagtacttaccagaaacagcacaggtagaaatatgcttaaattcgaacgtgtcattgacattttcaataagaaaaacaacggagcatggaatttctgcccgcaaaacgccggttattgggaacatatccctaagagtattacaaaattatcagatttaaaaatcgttggtttagatttctatatcactactgaagaatcaaaccgatttactgattttcctaaagactttaaaggtattgcaggttggatattagaagtaaaatcgaatacaccaggtaatacaacacaagtattaagacgtaataacttcccgtctgcacatcaatttttagttagaaactttggtactggtggcgttggtaaatggagtttattcgaaggaaaggtggttgaataatggtagtagataatttttcgaaagatgataacttaatcgagttacaaacaacatcacaatataatccggttattgacacaaacatcagtttctatgaatcagatagaggaactggtgttttaaattttgcagtaactaagaataacagacccttatctataagttctgaacatgttaaaacatctatcgtgttaaaaaccgatgattataacgtagatagaggcgcttatatttcagacgaattaacgatagtagacgcaattaatgggcgtttgcagtatgtgataccgaatgaatttttaaaacattcaggcaaggtgcatgctcaggcattctttacacaaaacgggagtaataatgttgttgttgaacgtcaatttagcttcaatattgaaaatgatttagttagtgggtttgatggtataacaaagcttgtttatatcaaatctattcaagatactatcgaagctgtcggtaaagattttaaccaattaaagcaaaatatggctgatacacaaacgttaatagcaaaagtgaatgatagtgcgacaaaaggcattcaacaaatcgaaatcaagcaaaacgaagctatacaagctattactgcgacgcaaactagtgcaacacaagctgttacagctgaattcgataaaatagttgaaaaagagcaagcgatttttgaacgtgttaacgaagttgaacaacaaatcaatggcgctgaccttgttaaaggtaattcaacaacgaattggcaaaagtctaaacttacagatgattacggtaaagcaattgaatcgtatgagcagtccatagatagcgttttaagcgcagttaacacatctaggattattcatattactaatgcaacagatgcgccagaaaagacggatataggcacgttagagaagcctggacaagatggtgttgatgacggttcttcgttcgatgaatcaacttatacatcaagcaaatctggtgtgttagttgtttatgttgttgataataatactgctcgtgcaacatggtacccagacgattcaaacgatgagtacacaaaatacaaaatctacggcacatggtacccgttttataaaaagaatgatggaaacttaactaagcaatttgttgaagaaacgtctaacaacgctttaaatcaagctaagcagtatgtagatgataaattcggaacaacgagctggcaacaacataagatgacagaggcgaatggtcaatcaattcaagttaacttaaataatgcgcaaggcgatttgggatatttaactgctggtaattactatgcaacaagagtgccggatttaccaggtagcgttgaaagttatgagggttatttatcggtattcgttaaagatgatacaaacaagctatttaacttcacaccttataactctaaaaagatttacacacgatcaatcacaaacggcagacttgagcaacagtggacagttcctaatgaacataaatcaacggtattgttcgacggtggcgcaaatggtgtaggtacaacaatcaatctaactgaaccgtacacaaactattctattttgttggtaagtggaacttatccaggtggcgttattgagggattcggactaaccgcattacctaacgcgattcaattgagtaaagcgaatgtagttgactcagacggcaacggtggcggtatttatgagtgcttactatccaaaacaagtagcactactttaagaatagataacgatgtgtactttgatttaggtaaaacatcaggttctggagcgaatgccaacaaagttactataactaaaattatggggtggaaataatgaaaatcacagtaaacgataaaaacgaagttatcggattcgttaatactggcggtttacgcaatagtttagatgtagatgataacaatgtgcctattaaatttaaagaagagttcgaacctagaaagtttgttttcactaacggcgaaattaaatacaatagcaatttcgaaaaagaagacgtaccgaatgcatcaaaccaacaaagtgcgtcagatttaagtgatgaggaacttcgcggaatggttgcgagtatgcaaatgcaggtggcacaagtaaacgtattaacaatggaattagctcaacaaaacgctatgttaacacaacagttgactgaactgaaaactaacaaaacaagtactgagggggacgtttaaataatgaagatgatttatccaacttttaaagacattaaaactttttatgtttggggttactataaaaacgagcaaattaagtggtacgtagacaagggtttaatcgataaagaagaatacgctttaatcactggagaaaaatatccagaaacaaaagatgaaaagtcacaggtgtaatgcttgtggctttttaatttgaataaagtgggtggcataatgtttggatttaccaaacgacatgaacaagattggcgtttaacgcgattagaagaaaatgataagactatgtttgaaaaattcgacagaatagaagatagtcttagagcgcaagaaaagatttatgacaaattagatagaaattttgaagaattaaagcgcgacaaggtagaagatgaaaagaataaagaaaagaatgccaagaatattagagacataaaaatgtggattctaggtttgatagggactatcttcagtacgattgtcatagctttactaagaactgtttttggtatttaaaggaggtgattaccatgcttaaagggattttaggatatagcttctgggcgtgcttctggtttggtaaatgtaaataacagttaagagtcagtgcttcggcactggctttttattttgattgaaatgaggtgcatacatgggattacctaatccgaaaaatagaaagcccacagctagtgaagtggttgaatgggcgttatatatcgctaaaaacaaaatagctattgatgtacctggttctggaatgggagcacaatgctgggatttacctaattatttactcgataaatattgggggtttagaacatggggaaatgctgatgctatggctcaaaaatccaattatagaggtagagatttcaagataattagaaatacaaaagattttgtaccacaaccaggcgactggggtgtttggactggtggttgggcaggacatgtaaacattgtagtgggaccatgcacaaaagactattggtatggcgtagatcaaaactggtatacaaataacgcaacaggaagtccaccttataaaattaaacactcttatcatgatggaccaggtggaggggttaaatattttgttagaccaccatatcatccagacaaaactacaccggcacctaaaccagaagatgatagtgatgataacgaaaaaaataataaaaaagttccaatttggaaagatgtaacaactataaagtacactatttctagccaagaggttaattatccagaatatatttatcactttatagtagaaggtaatcgacgactcgaaaaacctaaaggaataatgattagaaacgcacaaacgatgagctcggtagaaagtttatataacagtaggaagaaatacaaacaggatgtagaatatccccacttttatgttgatagacataatatttgggcacctagaagagctgtatttgaagttcctaatgaacctgattatatagttatagacgtatgtgaagattatagtgcgagtaaaaatgaatttatttttaatgagattcacgcaatggttgtagctgtagatatgatggccaaatatgagatacctctaagtattgaaaatttaaaagtagacgacagcatttggcgttcaatgttggaacatgttaattggaatatgattgacaacggtgttcctcctaaagataaatacgaagcattagaaaaggcattacttaatatatttaaaaacagagaaaaattattaaattctataactaagccaacagtaacaaaatctagaataaaagttatggtagataataaaaacgctgatatagctaatgtaagagactcgtcaccaacagccaacaatggttcggcatctaaacaaccgcagatcataacagaaacgagtccttatacattcaaacaagcactggataaacaaatggcaagaggtaacccgaaaaaatctaatgcttggggttgggctaacgctacacgagcacaaacgagttcagcaatgaatgtaaagcgtatatgggaaagtaacacacaatgctaccaaatgcttaatttaggcaagtatcaaggtgtttcagttagcgcacttaataagatacttaaaggtaagggaacattgaataatcaaggtaaagcgttcgcagaagcttgtaaaaagcacaacattaatgaaatttatttaatcgcgcatgctttcttagaaagtggatatggaacaagtaacttcgctaacggaaaagatggagtatacaactacttcggcattggcgcttacgacaacaatcctaactacgcaatgacgtttgcaaggaataaaggttggacatctccagcaaaagcaatcatgggcggtgctagcttcgtaagaaaggattacatcaataaaggtcaaaacacattgtaccgaattagatggaatcctaagaatccagctacccaccaatacgctactgctatagagtggtgccaacatcaagcaagtacaatcgctaagttatataaacaaatcggcttaaaaggtatctacttcacaagggataaatataaataaagaggtgtgtaaatgtacaaaataaaagatgttgaaacgagaataaaaaatgatggtgttgacttaggtgacattggctgtcgattttacactgaagatgaaaatacagcatctataagaataggtatcaatgacaaacaaggtcgtatcgatctaaaagcacatggcttaacacctagattacatttgtttatggaagatggctctatattcaaaaatgagccccttattatcgacgatgttgtaaaaggtttccttacctacaagatacctaaaaaggttatcaaacacgctggttatgttcgctgtaagctgtttttagagaaagaagaagaaaaaatacatgtcgcaaacttttctttcaatatcgttgatagtggtattgaatctgctgtagcaaaagaaatcgatgttaaattggtagatgatgctattacgagaattttaaaagataacgcgacagatttattgagcaaagactttaaagagaaaatagataaagatgtcatttcttacatcgaaaagaatgaaagtagatttaaaggtgcgaaaggtgataaaggtgaaccgggacaacctggagcaaaaggtgaagcaggtaaaaaaggagaacaaggcgcacccggtaaaaacggtactgtagtatcaatcaatcctgacactaaaatgtggcaaattgatggtaaagatacagatatcaaagcagaacctgagttattggacaaaatcaatatcgcaaatgttgaagggttagaaaataaattgcaagaagttgaaaaaatcaaagatacaactctcaacgactctaaaacgtatacggatacaaaaattgctgaactagttgatagcgcgcctgaatctatgaacacattaagagaattagcagaagcaatacaaaacaactctatttcagaaagtgtattgcaacagattggctcaaaagttaatacagaagattttgaggaattcaaacaaacactaaatgatttatatgctccaaaaaatcataatcatgacgagcggtatgttttgtcatctcaagcttttactaaacaacaagcggataatttatatcaactaaaaagcgcatctcaaccgacggttaaaatttggacaggaacagaaaatgaatataactatatatatcaaaaagacccgaatacgttatatttaattaaagggtgatttttatggaaggtaattttaaaaatgtaaagaagtttatttacgaaggtgaagaatatacaaaagtatatgctggaaatatccaagtatggaaaaagccttcatcttttgtaataaaacccttacctaaaaataaatatccggatagcatagaagaatcaacagcaaaatggacaataaatggagttgaacccaataaaagttatcaggtgacaatagaaaatgtacgtagcggtataatgaggatttcgcaaactaatttagggtcaagtgatttaggaatatcaggagtcaatagcggagttgcaagtaaaaatatcaactttagtaatccttcagggatgttgtacgtcactataagtgatgtttattcaggatctccgacattgaccattgaataattttaaacgactaatttttagtcgtttttttattttggataaaaggagcaaacaaatggatattaactggaaattgagattcaaaaacaaagcagtactaactggtttagttggagcattgttgctatttatcaagcaagtcacggatttattcggattagatttatctactcaattaaatcaagctagcgcaattataggcgctatcctcacgttacttacaggtattggcgttattactgacccaacgtcaaaaggcgtctcagattcatctatagcacagacatatcaagcgcctagagatagcaaaaaagaagaacaacaagttacgtggaaatcatcacaagacagtagtttaacgccggaattaagcgcgaaagcaccaaaagaatatgatacatcacaacctttcacagacgcctctaacgatgttggctttgatgtgaatgagtatcatcatggaggtggcgacaatgcaagcaaaattaactaaaaatgagtttatagagtggttgaaaacttctgagggaaaacaattcaatgtggacttatggtatggatttcaatgctttgattatgccaatgctggttggaaagttttgtttggattacttctaaaaggtttaggtgcaaaagatattccgttcgctaacaacttcgacggattagctactgtataccaaaatacaccggacttcttagcacaacctggcgacatggtggtattcggtagcaactacggtgctggatatggtcacgttgcatgggtaattgaagcaactttagattacatcattgtatatgagcagaattggctaggcggtggctggactgacggaatcgaacaacccggctggggttgggaaaaagttacaagacgacaacatgcttatgatttccctatgtggtttatccgtccgaattttaaaagtgagacagcgccacgatcagttcaatctcctacacaagcacctaaaaaagaaacagctaagccacaacctaaagcagtagaacttaaaatcatcaaagatgtggttaaaggttatgacctacctaagcgtggtagtaaccctaaaggtatagttatacacaacgacgcagggagcaaaggggcgactgctgaagcatatcgtaacggattagtaaatgcacctttatcaagattagaagcgggcattgcgcatagttacgtatcaggcaacacagtttggcaagccttagatgaatcacaagtaggttggcataccgctaatcaaataggtaataaatattattacggtattgaagtatgtcaatcaatgggcgcagataacgcgacattcttaaaaaatgaacaggcaactttccaagaatgcgctagattgttgaaaaaatggggattaccagcaaacagaaatacaatcagattgcacaatgaatttacttcaacatcatgccctcatagaagttcggttttacacactggttttgacccagtaactcgcggtctattgccagaagacaagcggttgcaacttaaagactactttatcaagcagattagggcgtacatggatggtaaaataccggttgccactgtctctaatgagtcaagcgcttcaagtaatacagttaaaccagttgcaagtgcatggaaacgtaataaatatggtacttactacatggaagaaagtgctagattcacaaacggcaatcaaccaatcacagtaagaaaagtggggccattcttatcttgtccagtgggttatcagttccaacctggtgggtattgtgattatacagaagtgatgttacaagatggtcatgtttgggtaggatatacatgggaggggcaacgttattacttgcctattagaacatggaatggttctgccccacctaatcagatattaggtgacttatggggagaaatcagttagaatgacatagtcatgtctatttaagcaggtgcgttacatacctgctttctatttacatttaaagataaaatgtgctattattttactagaactttttaacatttctctcaagatttaaatgtagataacaggcaggtactacggtacttgcctatttttttatgcaaattttaaaaaacactttactaataaacatttgtttagtataattatatttgtaggttagttgatgacttacaaattatgtgtaaggaggtgaaaagcctcatgctagacataataaaaacacttctagaacatcaagtattggcagtactgataattccagaagtgttaaaacaacttagagaatggcatctcggctacctagaccgaaagccaaacaacaaagattaacattatgcttggagcctgatggctcctccttacacttatataatataatattatttggaggttttcaattatgacagaacaaatgtatttaatattgtttttattaagcctaccattgttattatttatcgggagaaagacacatttttattgtttagataaaaagaatggacgtagataatatgagtgattataaattaaaaataattgaattgatcaaaagtgatataacaggttaccaaattcacaaacaaactggcgtagcgcaatatgtaatttcacaattaaggcaaggaaagcgcgaagtagataacttaactttaaatacaactgaaaaactatacagttacgcacgacaagtgttataatataaatgtgaaatggtcattcttgaaatgactcggtcgctactggcacagaccgtttaaagtgtcaccacaacatgaactgagaattcatatgacgttgctgacgagcgacaaagctctgtgttcctgaatgggagtaggtttgtgtggtggtataatttagtaacagcatagactgtctatagcaaagttgccgaagagattctaaacgtatttataaatacgtggcccttgctagataaccgcatcttaactgatgcggttatttttatccccacacaaccaacaaaaccacaccacctattaatttaggagtgtggttgttttaatatgtgaagctaaaataactacaaatgataccatttttgataccattttgttgtaaaacagaaaaaataaggaaaataaaaaaggcaaaaaaacgcattaaatcaacgtttattgtctcatgaaatttaaatgtatataaatttca

SEQ ID NO: 18 - DNA, 18 bp, synthetic construct
  misc_feature 1..18: note = Synthetic Construct
  source 1..18: mol_type = other DNA
aaagaagaacaataatat

SEQ ID NO: 19 - DNA, 18 bp, synthetic construct
  misc_feature 1..18: note = Synthetic Construct
  source 1..18: mol_type = other DNA
aaagaagaacaataatat

SEQ ID NO: 20 - DNA, 18 bp, synthetic construct
  misc_feature 1..18: note = Synthetic Construct
  source 1..18: mol_type = other DNA
taattattcccactcaat

SEQ ID NO: 21 - DNA, 18 bp, synthetic construct
  misc_feature 1..18: note = Synthetic Construct
  source 1..18: mol_type = other DNA
taattattcccactcgat

SEQ ID NO: 22 - DNA, 15 bp, synthetic construct
  misc_feature 1..15: note = Synthetic Construct
  source 1..15: mol_type = other DNA
tcccgccgtctccat

SEQ ID NO: 23 - DNA, 15 bp, synthetic construct
  misc_feature 1..15: note = Synthetic Construct
  source 1..15: mol_type = other DNA
tcccgccgtctccat

SEQ ID NO: 24 - DNA, 15 bp, synthetic construct
  misc_feature 1..15: note = Synthetic Construct
  source 1..15: mol_type = other DNA
tcccgccgtctccat

SEQ ID NO: 25 - DNA, 15 bp, synthetic construct
  misc_feature 1..15: note = Synthetic Construct
  source 1..15: mol_type = other DNA
tcccgccgtctccat

SEQ ID NO: 26 - DNA, 17 bp, synthetic construct
  misc_feature 1..17: note = Synthetic Construct
  source 1..17: mol_type = other DNA
ttatttagcaggaataa

SEQ ID NO: 27 - DNA, 17 bp, synthetic construct
  misc_feature 1..17: note = Synthetic Construct
  source 1..17: mol_type = other DNA
ttatttagcaggaataa

SEQ ID NO: 28 - DNA, 17 bp, synthetic construct
  misc_feature 1..17: note = Synthetic Construct
  source 1..17: mol_type = other DNA
ttatttagcaggaataa

SEQ ID NO: 29 - DNA, 22 bp, synthetic construct
  misc_feature 1..22: note = Synthetic Construct
  source 1..22: mol_type = other DNA
gttttaccatcattcccggcat

SEQ ID NO: 30 - DNA, 21 bp, synthetic construct
  misc_feature 1..21: note = Synthetic Construct
  source 1..21: mol_type = other DNA
attttacatcattcctggcat

SEQ ID NO: 31 - DNA, 21 bp, synthetic construct
  misc_feature 1..21: note = Synthetic Construct
  source 1..21: mol_type = other DNA
atgccaggtatgatgtaaaac

SEQ ID NO: 32 - DNA, 21 bp, synthetic construct
  misc_feature 1..21: note = Synthetic Construct
  source 1..21: mol_type = other DNA
gttttacatcattcctggcat

SEQ ID NO: 33 - DNA, 43 bp, synthetic construct
  misc_feature 1..43: note = Synthetic Construct
  source 1..43: mol_type = other DNA
gttttaccatcattcccggcatgttttacatcattcctggcat